(19) United States
(12) Patent Application Publication
(10) Pub. No.: US 2003/0176314 A1
(43) Pub. Date: Sep. 18, 2003

(54) Compounds for the treatment of pain

(76) Inventors: Carlos C. Forray, Paramus, NJ (US); Joel Kawakami, Newfoundland, NJ (US); Michael J. Konkel, Garfield, NJ (US); Lakmal W. Boteju, Cedar Grove, NJ (US); John M. Wetzel, Fairlawn, NJ (US); Stewart A. Noble, Lake Forest, IL (US); Honghe Wan, Kearny, NJ (US)

Correspondence Address: Cooper & Dunham LLP, 1185 Avenue of the Americas, New York, NY, 10036, US

(21) Appl. No.: 10/253,237

(22) Filed: Sep. 24, 2002

Related U.S. Application Data
(60) Provisional application No. 60/324,767, filed on Sep. 24, 2001.

(51) Int. Cl. A61K 31/00

(52) U.S. Cl. 514/1

(57) ABSTRACT

This invention provides methods of treating pain, urinary incontinence and other abnormalities mediated by a NPFF receptor, which comprises administering to a subject a therapeutically effective amount of a chemical compound which acts at the NPFF1 receptor, the NPFF2 receptor, or at both the NPFF1 and NPFF2 receptors.

Throughout this application, various publications are referenced within parentheses. Disclosures of these publications in their entireties are hereby incorporated by reference into this application to more fully describe the state of the art to which this invention pertains. Full bibliographic citations for these references may be found immediately preceding the claims.

### BACKGROUND OF THE INVENTION

Neuroregulators comprise a diverse group of natural products that subserve or modulate communication in the nervous system. They include, but are not limited to, neuropeptides, amino acids, biogenic amines, lipids and lipid metabolites, and other-metabolic byproducts. These neuroregulators interact with one or more specific types of cell surface receptors to activate one or more biological responses from within the cell by transducing signals from the receptor to the inside of the cell. G-protein coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequel such as stimulation of adenylyl cyclase.

Neuropeptide FF (NPFF) is an octapeptide isolated from bovine brain in 1985 by Yang et al. using antibodies to the molluscan neuropeptide FMRFamide (FMRFa). FMRFamide-like immmunoreactivity was observed in rat brain, spinal cord, and pituitary, suggesting the existence of mammalian homologs of the FMRFa family of invertebrate peptides. The isolation of NPFF, named for its N- and C-terminal phenylalanines and another mammalian peptide, NPAF, confirmed the existence of a mammalian family of peptides sharing the C-terminal homology with FMRFa (Yang et al. 1985). NPFF is also called F8Famide and morphine modulating peptide, whereas NPAF is also called A18Famide in the literature. Molecular cloning has revealed that NPFF and NPAF are encoded from the same gene, and cleaved from a common precursor protein (Vilim and Ziff 1995). Studies of the localization, radioligand binding, and function of NPFF-like peptides indicate they are neuromodulatory peptides whose effects are likely to be mediated by G protein-coupled receptors (see PCT International Publication No. WO 00/18438).

There are two known receptor subtypes for NPFF, NPFF-1 and NPFF-2 (Bonini et al. 2000). Recently, two NPFF receptor subtypes (NPFF-1 and NPFF-2) were discovered and cloned from rat and human tissues (PCT International Publication No. WO 00/18438). The localization of protein and mRNA for these two receptors indicates that they may have utility as targets for drugs to treat a variety of disorders including, but not limited to, disorders of electrolyte balance, diabetes, respiratory disorders, gastrointestinal disorders, depression, phobias, anxiety, mood disorders, cognition/memory disorders, obesity, pain, alertness/sedation, lower urinary tract disorders and cardiovascular indications.

NPFF is an endogenous modulator of opioid systems with effects on morphine analgesia, tolerance, and withdrawal (Panula et al. 1996 Roumy and Zajac, 1998). NPFF appears to represent an endogenous “anti-opioid” system in the CNS, acting at specific high-affinity receptors that are distinct from opioid receptors (Payza et al. 1993, Raffa et al. 1994). Endogenous NPFF has been suggested to play a role in morphine tolerance: agonists of NPFF precipitate “morphine abstinence syndrome” (symptoms of morphine withdrawal) in morphine-dependent animals (Malin et al. 1990, 1993) while antagonists and anti-NPFF IgG restore morphine sensitivity and ameliorate symptoms of withdrawal. NPFF has also been shown to participate in the regulation of pain threshold, showing both “anti-opiate” effects and analgesic effects, depending on the test system (Panula et al. 1996, Roumy and Zajac, 1998).

The ability of NPFF peptides to modulate the opioid system raised the possibility that NPFF interacts directly with opiate receptors. However, radioligand binding assays using a tyrosine-substituted NPFF analog [¹²⁵I]Y8Fa demonstrate that NPFF acts through specific high affinity binding sites distinct from opiate receptors (Allard et al. 1989, 1992, Gouarderes et al. 1998, Panula at al. 1987) that are sensitive to inhibition by guanine nucleotides (Payza et al. 1993).

NPFF and related peptidic agonists exhibit direct analgesic activity in some animal models. NPFF has been shown to produce analgesia in the rat tail-flick and paw pressure models, upon intrathecal administration (Gouarderes et al. 1993). Similarly, a NPFF-like peptide, SLAAPQRF-amide, isolated from rat brain and spinal cord (Yang and Martin, 1995) produces antinociceptive action in the tail-flick and paw pressure models (Jhamadas et al. 1996). NPFF has also been observed to play a role in animal models of chronic pain. For example, NPFF has recently been shown to be involved in inflammatory pain (Kontinen et al. 1997) and neuropathic pain (Wei et al. 1998). Importantly, NPFF was shown to attenuate the allodynia associated with neuropathic pain, suggesting that it may be clinically useful in treating this condition. NPFF also has been shown to produce nighttime hyperasthesic analgesia in the tail-flick test upon i.c.v. administration in the rat (Oberling et al. 1993). A synthetic NPFF analog, (D)Tyr¹, (NMe)Phe³-NPFF (1DMe, 1DMeY8Fa), which is partially protected against enzymatic degradation and also has high affinity for NPFF receptors, shows long-lasting analgesic activity in the above models upon intrathecal administration (Gouarderes et al. 1996a,b). In carrageenan inflammation, 5-10 mmol of 1DMe was effective against both thermal hyperalgesia and mechanical allodynia, and in a neuropathic pain model, 1DMe showed antiallodynic effects against cold allodynia (Xu et al. 1999). 1DMe also shows analgesic activity in the rat vocalization threshold upon intrathecal administration (Coudore et al. 1997).

Recent studies in our laboratories have shown that NPFF also has peripheral effects. NPFF and related agonists show decrease in the contraction frequency of the rat bladder upon i.v. and i.t. administration (see PCT International Publication No. WO 00/18438). A potent NPFF agonist, PFRF-amide, has been shown to increase blood pressure and heart rate in rats (Huang et al. 2000).

In addition, NPFF and related peptides have a number of other biological activities that may be therapeutically relevant. NPFF and FMRFamide have been shown to reduce deprivation- and morphine-induced feeding in rats (Kavaliers et al. 1985, Murase et al. 1996, Robert et al. 1989), indicating that NPFF receptors may be important targets in the treatment of eating disorders. Effects on feeding behavior are further supported by findings that demonstrate NPFF-like immunoreactive neurons, as well as NPFF1 receptor mRNA, localize to the hypothalamus (Panula, et al. 1996, Bonini at al, 2000). The NPFF 1-selective ligand, BIBP 3226, which is also a neuropeptide Y Y1 antagonist, blocks feeding through a nonspecific mechanism, not secondary to inhibition of Y1 (Morgan et al. 1998). These data suggest that feeding behavior may be regulated through a NPFF1 receptor mechanism. FMRFamide has also been shown to produce antipsychotic (Muthal et al. 1997) and antianxiety (Muthal and Chopde, 1994) effects in rats, indicating that NPFF receptors may be valuable targets for the treatment of psychosis and anxiety. There is evidence for a role of NPFF in learning and memory. Kavaliers and Colwell (1993) have shown that i.c.v. administered NPFF has a biphasic effect of spatial learning in mice: low doses improve and high doses impair learning. This suggests the possibility that different NPFF receptor subtypes may have opposite roles in some types of learning behavior. NPFF is known to have indirect effects on water and electrolyte balance. Arima et al. (1996) have shown that NPFF will reduce the increase in vasopressin release produced by salt loading or hypovolemia. Additionally, NPFF may be involved in the control of plasma aldosterone levels (Labrouche et al., 1998). These observations raise the possibility that agents targeting NPFF receptors may be of value in the treatment of diuresis or in the treatment of cardiovascular conditions such as hypertension and congestive heart failure. Drugs acting at NPFF receptors may be of value in the treatment of diabetes, since NPFF and A-18-Famide have been shown to produce significant inhibition of glucose- and arginine-induced insulin release in rats (Fehmann et al. 1990). Several investigators have reported effects of NPFF and analogs on intestinal motility in mice (Gicquel et al. 1993) and guinea pigs (Demichel et al. 1993, Raffe and Jacoby 1989).

When administered to isolated preparations of guinea pig ileum, the actions of NPFF oppose those of opioids.

Conversely, i.c.v. administration of NPFF in mice produces effects similar to those of morphine on intestinal motility. Together, these results indicate a complex modulatory role for NPFF in intestinal motility, but indicate that NPFF receptors are potential targets for drugs to treat GI motility disorders, including irritable bowel syndrome. NPFF has been shown to precipitate nicotine abstinence syndrome in a rodent model, raising the possibility that nicotine dependence may be attenuated by measures which inactivate NPFF (Malin et al. 1996). Thus, NPFF receptor antagonists may be of use for this purpose. Finally, NPFF is known to elicit two acute cardiovascular responses when administered peripherally: elevation of blood pressure and heart rate (Allard et al. 1995, Laguzzi et al. 1996). These actions may be mediated peripherally, centrally, or both. Thus, agents acting at NPFF receptors may be of value in the treatment of hypertension or hypotension.

Described herein are unique sulfonamido-peptidomimetic ligands which are either agonists and/or antagonists at one or more NPFF receptor subtypes. Also described herein are quinazolino- and quinolino-guanidine containing compounds that are the first known small molecule (non-peptide/non-peptoid) ligands (either agonists and/or antagonists) at the neuropeptide NPFF1 and NPFF2 receptors.

It is evident that NPFF agonists and/or antagonists have great potential as being therapeutically useful agents for the treatment of a diverse array of clinically relevant human disorders. NPFF agonists may have therapeutic potential, among others, for the treatment of pain, memory loss, circadian rhythm disorders, and micturition disorders. Cloned receptor subtypes of NPFF and the development of high-efficiency in vitro assays, both for binding and receptor activation, has aided the discovery and development of novel NPFF ligands in our hands. Moreover, it is practically possible to design a molecule that is an agonist at one NPFF subtype, and an antagonist at the other(s). This concept of a dual-acting molecule provides an attractive means of designing drugs that can treat multiple disorders. These molecules may be used by themselves as drugs or as valuable tools for the design of drugs for the treatment of various clinical abnormalities in a subject wherein the abnormality is alleviated by increasing or decreasing the activity of a mammalian NPFF receptor which comprises administering to the subject an amount of a compound which is an antagonist or agonist of mammalian NPFF receptors to effect a treatment of the abnormality. The abnormality can be a lower urinary tract disorder, such as interstitial cystitis or urinary incontinence, such as urge incontinence or stress incontinence particularly urge incontinence, a regulation of a steroid hormone disorder, an epinephrine release disorder, a gastrointestinal disorder, irritable bowel syndrome, a cardiovascular disorder, an electrolyte balance disorder, diuresis, hypertension, hypotension, diabetes, hypoglycemia, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, an eating disorder, obesity, a serotonergic function disorder, an olfaction disorder, nasal congestion, a sympathetic innervation disorder, an affective disorder, pain, psychotic behavior, morphine tolerance, nicotine addiction, opiate addiction, or migraine.

### SUMMARY OF THE INVENTION

The present invention provides a method of treating pain in a subject which comprises administering to the subject an amount of a compound effective to treat pain in the subject, wherein the compound binds to a NPFF1 receptor with a binding affinity greater than ten-fold higher than the binding affinity with which the compound binds to a NPFF2 receptor.

The invention also provides a method of treating a urinary disorder in a subject which comprises administering to the subject an amount of a compound effective to treat the urinary disorder in the subject, wherein the compound binds to a NPFF1 receptor with a binding affinity greater than ten-fold higher than the binding affinity with which the compound binds to a NPFF2 receptor.

The present invention further provides a method of treating pain in a subject which comprises administering to the subject an amount of a compound effective to treat pain in the subject, wherein the compound binds to a NPFF2 receptor with a binding affinity greater than ten-fold higher than the binding affinity with which the compound binds to a NPFF1 receptor.

The invention also provides a method of treating a urinary disorder in a subject which comprises administering to the subject an amount of a compound effective to treat the urinary disorder in the subject, wherein the compound binds to a NPFF2 receptor with a binding affinity greater than ten-fold higher than the binding affinity with which the compound binds to a NPFF1 receptor.

### BRIEF DESCRIPTION OF THE FIGURES

FIGS. 1A-1B: Correlation between binding affinities at human and rat recombinant Neuropeptide FF (NPFF1 and NPFF2) receptors. The binding affinities (pKi values) for 18 compounds were tested at rat NPFF (rNPFF) receptors and plotted against the pKi values for the same 18 compounds tested at human NPFF (hNPFF) receptors. A slope value of 0.83 (r²=0.29) was obtained for rat NPFF1 vs. human NPFF1 (FIG. 1A) and a slope value of 0.75 (r²=0.61) was obtained for rat NPFF2 vs. human NPFF2 (FIG. 1B); both slope values indicate a positive correlation.

FIG. 2: Effect of compound 4006A on bladder activity in the anesthetized rat. Rhythmic elevations in bladder pressure, resulting from distension induced contractions, were unaffected by i.v. administration of physiological saline. In contrast, the NPFF receptor ligand compound 4006A produced immediate inhibition of bladder activity, which persisted for 12 min.

FIG. 3: Effect of compound 4005A on bladder activity in the anesthetized rat. Rhythmic elevations in bladder pressure, resulting from distension induced contractions, were unaffected by i.v. administration of physiological saline. In contrast, the NPFF receptor ligand compound 4005A produced immediate inhibition of bladder activity, which persisted for 35 min.

### DETAILED DESCRIPTION OF THE INVENTION

The present invention provides a method of treating pain in a subject which comprises administering to the subject an amount of a compound effective to treat pain in the subject, wherein the compound binds to a NPFF1 receptor with a binding affinity greater than ten-fold higher than the binding affinity with which the compound binds to a NPFF2 receptor.

In one embodiment of any of the methods described herein, the compound binds to the NPFF1 receptor with a binding affinity greater than 25-fold higher than the binding affinity with which the compound binds to a NPFF2 receptor. In a further embodiment, the compound binds to the NPFF1 receptor with a binding affinity greater than 50-fold higher than the binding affinity with which the compound binds to a NPFF2 receptor.

The invention also provides a method of treating a urinary disorder in a subject which comprises administering to the subject an amount of a compound effective to treat the urinary disorder in the subject, wherein the compound binds to a NPFF1 receptor with a binding affinity greater than ten-fold higher than the binding affinity with which the compound binds to a NPFF2 receptor. In one embodiment, the urinary disorder is urinary incontinence. In different embodiments, the urinary incontinence is urge incontinence or stress incontinence. In another embodiment, the urinary disorder is urinary retention.

In one embodiment, the compound binds to the NPFF1 receptor with a binding affinity greater than 25-fold higher than the binding affinity with which the compound binds to a NPFF2 receptor. In a further embodiment, the compound binds to the NPFF1 receptor with a binding affinity greater than 50-fold higher than the binding affinity with which the compound binds to a NPFF2 receptor.

The invention further provides a method of treating an abnormality mediated by a NPFF1 receptor in a subject which comprises administering to the subject an amount of a compound effective to treat the abnormality in the subject, wherein the compound binds to the NPFF1 receptor with a binding affinity greater than ten-fold higher than the binding affinity with which the compound binds to a NPFF2 receptor. In different embodiments, the abnormality is an eating disorder, obesity, a psychotic disorder, anxiety, a learning disorder, a memory disorder, an electrolyte balance disorder, diuresis, diabetes, an intestinal motility disorder, irritable bowel syndrome, nicotine addiction, or a cardiovascular disorder. In different embodiments, the abnormality is a lower urinary tract disorder, interstitial cystitis, a steroid hormone disorder, an epinephrine release disorder, a gastrointestinal disorder, hypoglycemia, a respiratory disorder, asthma, a reproductive function disorder, an immune disorder, an endocrine disorder, a musculoskeletal disorder, a neuroendocrine disorder, a cognitive disorder, a sensory modulation and transmission disorder, a motor coordination disorder, a sensory integration disorder, a motor integration disorder, a dopaminergic function disorder, an appetite disorder, a serotonergic function disorder, an olfaction disorder, nasal congestion, a sympathetic innervation disorder, an affective disorder, morphine tolerance, opiate addiction, or migraine.

In one embodiment, the compound binds to the NPFF1 receptor with a binding affinity greater than 25-fold higher than the binding affinity with which the compound binds to a NPFF2 receptor. In a further embodiment, the compound binds to the NPFF1 receptor with a binding affinity greater than 50-fold higher than the binding affinity with which the compound binds to a NPFF2 receptor.

In one embodiment of any of the methods described herein, the subject is a human being and the NPFF1 receptor is the human NPFF1 receptor and the NPFF2 receptor is the human NPFF2 receptor.

In one embodiment of any of the methods described herein, the compound is an agonist at the NPFF1 receptor and an agonist at the NPFF2 receptor. In one embodiment of any of the methods described herein, the compound is an antagonist at the NPFF1 receptor and an antagonist at the NPFF2 receptor. In one embodiment of any of the methods described herein, the compound is an agonist at the NPFF1 receptor and an antagonist at the NPFF2 receptor. In one embodiment of any of the methods described herein, the compound is an antagonist at the NPFF1 receptor and an agonist at the NPFF2 receptor.

In one embodiment of any of the methods described herein, the compound binds to the human NPFF1 receptor with a binding affinity at least 10-fold higher than the binding affinity with which the compound binds to each of a human α_{1A} adrenoceptor, a human α_{1B} adrenoceptor, and a human α_{1D} adrenoceptor.

In one embodiment of any of the methods described herein, the compound binds to the human NPFF1 receptor with a binding affinity at least 10-fold higher than the binding affinity with which the compound binds to each of a human α_{1A} adrenoceptor, a human α_{1P} adrenoceptor and a human α_{2C} adrenoceptor.

In one embodiment of any of the methods described herein, the compound binds to the human NPFF1 receptor with a binding affinity at least 10-fold higher than the binding affinity with which the compound binds to a human dopamine D₂ receptor.

In one embodiment of any of the methods described herein, the compound binds to the human NPFF1 receptor with a binding affinity at least 10-fold higher than the binding affinity with which the compound binds to a human histamine H₁ receptor.

In one embodiment of any of the methods described herein, the compound binds to the human NPFF1 receptor with a binding affinity at least 10-fold higher than the binding affinity with which the compound binds to a human NMDA receptor.

In one embodiment of any of the methods described herein, the compound binds to the human NPFF1 receptor with a binding affinity at least 10-fold higher than the binding affinity with which the compound binds to a human norepinephrine transporter or to a human serotonin transporter.

In one embodiment of any of the methods described herein, the compound binds to the human NPFF1 receptor with a binding affinity at least 10-fold higher than the binding affinity with which the compound binds to each of a human neuropeptide Y1 receptor, a human neuropeptide Y2 receptor, a human neuropeptide Y4 receptor, and a human neuropeptide Y5 receptor.

The invention also provides a method of treating pain in a subject which comprises administering to the subject an amount of a compound effective to treat pain in the subject, wherein the compound binds to a NPFF2 receptor with a binding affinity greater than ten-fold higher than the binding affinity with which the compound binds to a NPFF1 receptor.

In one embodiment of any of the methods described herein, the compound binds to the NPFF2 receptor with a binding affinity greater than 25-fold higher than the binding affinity with which the compound binds to a NPFF1 receptor. In a further embodiment, the compound binds to the NPFF2 receptor with a binding affinity greater than 50-fold higher than the binding affinity with which the compound binds to a NPFF1 receptor.

The invention also provides a method of treating a urinary disorder in a subject which comprises administering to the subject an amount of a compound effective to treat the urinary disorder in the subject, wherein the compound binds to a NPFF2 receptor with a binding affinity greater than ten-fold higher than the binding affinity with which the compound binds to a NPFF1 receptor. In one embodiment, the urinary disorder is urinary incontinence. In different embodiments, the urinary incontinence is urge incontinence or stress incontinence. In another embodiment, the urinary disorder is urinary retention.

In one embodiment, the compound binds to the NPFF2 receptor with a binding affinity greater than 25-fold higher than the binding affinity with which the compound binds to a NPFF1 receptor. In a further embodiment, the compound binds to the NPFF2 receptor with a binding affinity greater than 50-fold higher than the binding affinity with which the compound binds to a NPFF1 receptor.

The invention further provides a method of treating an abnormality mediated by a NPFF2 receptor in a subject which comprises administering to the subject an amount of a compound effective to treat the abnormality in the subject, wherein the compound binds to the NPFF2 receptor with a binding affinity greater than ten-fold higher than the binding affinity with which the compound binds to a NPFF2 receptor. In different embodiments, the abnormality is an eating disorder, obesity, a psychotic disorder, anxiety, a learning disorder, a memory disorder, an electrolyte balance disorder, diuresis, diabetes, an intestinal motility disorder, irritable bowel syndrome, nicotine addiction, or a cardiovascular disorder. In different embodiments, the abnormality is a lower urinary tract disorder, interstitial cystitis, a steroid hormone disorder, an epinephrine release disorder, a gastrointestinal disorder, hypoglycemia, a respiratory disorder, asthma, a reproductive function disorder, an immune disorder, an endocrine disorder, a musculoskeletal disorder, a neuroendocrine disorder, a cognitive disorder, a sensory modulation and transmission disorder, a motor coordination disorder, a sensory integration disorder, a motor integration disorder, a dopaminergic function disorder, an appetite disorder, a serotonergic function disorder, an olfaction disorder, nasal congestion, a sympathetic innervation disorder, an affective disorder, morphine tolerance, opiate addiction, or migraine.

In one embodiment, the compound binds to the NPFF2 receptor with a binding affinity greater than 25-fold higher than the binding affinity with which the compound binds to a NPFF1 receptor. In a further embodiment, the compound binds to the NPFF2 receptor with a binding affinity greater than 50-fold higher than the binding affinity with which the compound binds to a NPFF1 receptor.

In one embodiment, the subject is a human being and the NPFF1 receptor is the human NPFF1 receptor and the NPFF2 receptor is the human NPFF2 receptor.

In one embodiment, the compound is an agonist at the NPFF1 receptor and an agonist at the NPFF2 receptor. In one embodiment, the compound is an antagonist at the NPFF1 receptor and an antagonist at the NPFF2 receptor. In one embodiment, the compound is an agonist at the NPFF1 receptor and an antagonist at the NPFF2 receptor. In one embodiment, the compound is an antagonist at the NPFF1 receptor and an agonist at the NPFF2 receptor.

In one embodiment of any of the methods described herein, the compound binds to the human NPFF2 receptor with a binding affinity at least 10-fold higher than the binding affinity with which the compound binds to each of a human α_{1A} adrenoceptor, a human α_{1P} adrenoceptor, and a human α_{1D} adrenoceptor.

In one embodiment of any of the methods described herein, the compound binds to the human NPFF2 receptor with a binding affinity at least 10-fold higher than the binding affinity with which the compound binds to each of a human α_{1A} adrenoceptor, a human α_{2P} adrenoceptor and a human α_{2C} adrenoceptor.

In one embodiment of any of the methods described herein, the compound binds to the human NPFF2 receptor with a binding affinity at least 10-fold higher than the binding affinity with which the compound binds to a human dopamine D₂ receptor.

In one embodiment of any of the methods described herein, the compound binds to the human NPFF2 receptor with a binding affinity at least 10-fold higher than the binding affinity with which the compound binds to a human histamine H₁ receptor.

In one embodiment of any of the methods described herein, the compound binds to the human NPFF2 receptor with a binding affinity at least 10-fold higher than the binding affinity with which the compound binds to a human NMDA receptor.

In one embodiment of any of the methods described herein, the compound binds to the human NPFF2 receptor with a binding affinity at least 10-fold higher than the binding affinity with which the compound binds to a human norepinephrine transporter or to a human serotonin transporter.

In one embodiment of any of the methods described herein, the compound binds to the human NPFF2 receptor with a binding affinity at least 10-fold higher than the binding affinity with which the compound binds to each of a human neuropeptide Y1 receptor, a human neuropeptide Y2 receptor, a human neuropeptide Y4 receptor, and a human neuropeptide Y5 receptor.

In further embodiments of any of the methods described herein, the compound binds to a NPFF receptor with a binding affinity greater than 10-fold higher than the binding affinity with which it binds to any of the non-NPFF receptors described herein. In further embodiments of any of the methods described herein, the compound binds to a NPFF receptor with a binding affinity greater than 10-fold higher than the binding affinity with which it binds to a human norepinephrine transporter or to a human serotonin transporter. Examples of the binding characteristics of such compounds are shown in Table 8.

For certain compounds disclosed herein, enantiomers, diastereomers and double bond regioisomers and stereoisomers exist. This invention contemplates racemic mixtures of compounds as well as isolated enantiomers. This invention also contemplates mixtures of diastereomers, double bond regioisomers or stereoisomers as well as isolated diastereomers or double bond regioisomers or stereoisomers.

The small molecule compounds disclosed herein are the first known (non-peptide/non-peptoid) ligands (either antagonists or agonists) at the neuropeptide FF(NPFF) receptor(s).

The term “agonist” is used throughout this application to indicate a compound which increases the activity of any of the receptors of the subject invention. The term “antagonist” is used throughout this application to indicate a compound which binds to, but does not increase the activity of, any of the receptors of the subject invention.

The activity of a G-protein coupled receptor such as the polypeptides disclosed herein may be measured using any of a variety of functional assays in which activation of the receptor in question results in an observable change in the level of some second messenger system, including, but not limited to, adenylate cyclase, calcium mobilization, arachidonic acid release, ion channel activity, inositol phospholipid hydrolysis or guanylyl cyclase. Heterologous expression systems utilizing appropriate host cells to express the nucleic acid of the subject invention are used to obtain the desired second messenger coupling. Receptor activity may also be assayed in an oocyte expression system.

As used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

The formulations of the present invention can be solutions, suspensions, emulsions, syrups, elixirs, capsules, tablets, and the like. The compositions may contain a suitable carrier, diluent, or excipient, such as sterile water, physiological saline, glucose, or the like. Moreover, the formulations can also be lyophilized, and/or may contain auxiliary substances, such as wetting or emulsifying agents, pH buffering agents, adjuvants, gelling or viscosity enhancing additives, preservatives, flavoring agents, colors, and the like, depending upon the route of administration and the preparation desired. Standard texts, such as “Remington's Pharmaceutical Science”, 17th Ed., 1985, incorporated herein by reference, may be consulted to prepare suitable preparations, without undue experimentation.

The formulations can include powdered carriers, such as lactose, sucrose, mannitol, starch, cellulose derivatives, magnesium stearate, stearic acid, and the like. Further, tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. The formulations can also contain coloring and flavoring to enhance patient acceptance. The formulations can also include any of disintegrants, lubricants, plasticizers, colorants, and dosing vehicles.

In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents, and, if necessary, buffer substances.

Antioxidants such as, for example, sodium bisulfate, sodium sulfite, citric acid and its salts, sodium EDTA, ascorbic acid, and the like can be used either alone or in combination with other suitable antioxidants or stabilizing agents typically employed in the pharmaceutical compositions. In addition, parenteral solutions can contain preservatives, such as, for example, benzalkonium chloride, methyl- or propyl-paraben, chlorobutanol and the like.

The term “therapeutically effective amount” as used herein means that amount of a compound that elicits the biological or medicinal response in a tissue, system, animal or human that is being sought by a researcher, veterinarian, medical doctor or other clinician, which includes alleviation of the symptoms of the disease, disorder, or abnormality being treated.

The term “subject,” as used herein refers to an animal, preferably a mammal, most preferably a human, who has been the object of treatment, observation or experiment.

In order for a composition to be administered to an animal or human, and for any particular method of administration, it is preferred to determine the toxicity in a suitable animal model; the dosage of the composition(s), and the concentration of components in the composition; and the timing of administration in order to maximize the response. Such determinations do not require undue experimentation from the knowledge of the skilled artisan, the present disclosure and the documents cited herein.

The present invention includes within its scope prodrugs of the compounds of this inventions. In general, such prodrugs will be functional derivatives of the compounds of the invention which are readily convertible in vivo into the required compound. A prodrug of the quinazolino- and quinolino-guanidines may have an acyl group attached to any of the three nitrogens of the guanidine, forming an N-acyl guanidine.

Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985.

Included in this invention are pharmaceutically acceptable salts and complexes of all of the compounds described herein. The salts include, but are not limited to, the following acids and bases: Inorganic acids which include hydrochloric acid, hydrofluoric acid, hydrobromic acid, hydroiodic acid, sulfuric acid, and boric acid; organic acids which include acetic acid, trifluoroacetic acid, formic acid, oxalic acid, malonic acid, succinic acid, fumaric acid, tartaric acid, maleic acid, citric acid, methanesulfonic acid, trifluoromethanesulfonic acid, benzoic acid, glycolic acid, lactic acid, and mandelic acid; inorganic bases include ammonia and hydrazine; and organic bases which include methylamine, ethylamine, hydroxyethylamine, propylamine, dimethylamine, diethylamine, trimethylamine, triethylamine, ethylenediamine, hydroethylamine, morpholine, piperazine, and guanidine.

This invention further provides for the hydrates and polymorphs of all of the compounds described herein.

The present invention further includes metabolites of the compounds of the present invention. Metabolites include active species produced upon introduction of compounds of this invention into the biological milieu.

One skilled in the art will readily appreciate that appropriate biological assays can be used to determine the therapeutic potential of the claimed compounds for treating the disorders noted herein.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

I. NPFF Receptors

Cloning of Rat and Human NPFF1 Receptor

MOPAC (Mixed Oligonucleotide Primed Amplification of cDNA

100 ng of rat genomic DNA (Clonetech, Palo Alto, Calif.) was used for degenerate MOPAC PCR using Taq DNA polymerase (Boehringer-Mannheim, Indianapolis, Ind.) and the following degenerate oligonucleotides: JAB126, designed based on an alignment of the sixth transmembrane domain of more than 180 members of the rhodopsin superfamily of G protein-coupled receptors; and JAB108, designed based on an alignment of the seventh transmembrane domain of the same rhodopsin superfamily.

The conditions for the MOPAC PCR reaction were as follows: 3 minute hold at 94° C.; 10 cycles of 1 minute at 94° C., 1 minute 45 seconds at 44° C., 2 minutes at 72° C.; 30 cycles of 94° C. for 1 minute, 49° C. for 1 minute 45 seconds, 2 minutes at 72° C.; 4 minute hold at 72° C.; 4° C. until ready for agarose gel electrophoresis.

The products were run on a 1% agarose TAE gel and bands of the expected size (^{˜}150 bp) were cut from the gel, purified using the QIAQUICK gel extraction kit (QIAGEN, Chatsworth, Calif.), and subcloned into the TA cloning vector (Invitrogen, San Diego, Calif.). White (insert-containing) colonies were picked and subjected to PCR using pCR2.1 vector primers JAB1 and JAB2 using the Expand Long Template PCR System and the following protocol: 94° C. hold for 3 minutes; 35 cycles of 94° C. for 1 minute, 68° C. for 1 minute 15 seconds; 2 minute hold at 68° C., 4° C. hold until products were ready for purification. PCR products were purified by isopropanol precipitation (10 μl PCR product, 18 μl low TE, 10.5 μl 2M NaClO₄ and 21.5 μl isopropanol) and sequenced using the ABI Big Dye cycle sequencing protocol and ABI 377 sequencers (ABI, Foster City, Calif.). Nucleotide and amino acid sequence analyses were performed using the Wisconsin Package (GCG, Genetics Computer Group, Madison, Wis.). Two PCR products produced from rat genomic cDNA (MPR3-RGEN-31 and MPR3-RGEN-45) were determined to be identical clones of a novel G protein-coupled receptor-like sequence based on database searches and its homology to other known G protein-coupled receptors (^{˜}30-40% amino acid identity to dopamine D2, orexin, galanin, angiotensin 1 and 5-HT_{2b} receptors). This novel sequence was designated SNORF2.

Cloning of the Full-Length Coding Sequence of SNORF2 (Rat NPFF1)

Pools of the rat hypothalamic cDNA library “I” were screened by PCR with SNORF2-specific primers JAB208 and JAB209 and the Expand Long Template PCR system (Boehringer-Mannheim, Indianapolis, Ind.) with the following PCR protocol: 94° C. hold for 3 minutes; 40 cycles of 94° C. for 1 minute, 68° C. for 2 minutes; 4 minute hold at 68° C.; 4° C. hold until the samples are run on a gel. This screen yielded a positive pool 136E and a positive sub-pool 136E-17. High stringency hybridization of isolated colonies from 136E-17 with the SNORF2-specific oligonucleotide probe JAB211 and subsequent PCR testing of positive colonies indicated that the isolated clone 136E-17-lB-1 contained at least a partial clone of SNORF2. Sequencing of 136E-17-1B-1 revealed that this insert contained the coding region from the TMIII-TMIV loop through the stop codon, including some 3′ untranslated sequence. From this sequence, a new forward primer, JAB221, was designed in TMV. PCR screening of a second rat hypothalamic cDNA library “J” with primers JAB221 and JAB209, and subsequent colony hybridization with the JAB211 probe on a low complexity positive sub-pool resulted in the isolation of a SNORF2 clone J-13-16-A1. Full-length double-stranded sequence of SNORF2 was determined by sequencing both strands of the J-13-16-Al plasmid using an ABI 377 sequencer as described above. This insert is about 2.8 kb in length with an approximately 200 bp 5′ untranslated region, a 1296 bp coding region, and a 1.3 kb 3′untranslated region. The clone is also in the correct orientation for expression in the mammalian expression vector pEXJ.T7. This construct of SNORF2 in pEXJ.T7 was designated BN-6. The full length SNORF2 was determined to be most like the orexin 1 receptor (45% DNA identity, 35% amino acid identity), orexin 2 receptor (40% DNA identity, 32% amino acid identity), and NPY2 receptor (47% DNA identity, 29% amino acid identity), although several other G protein-coupled receptors also displayed significant homology. There were no sequences in the Genbank databases (genembl, sts, est, gss, or swissprot) that were identical to SNORF2. SNORF2 also showed significant homology (85% nucleotide identity, 93% amino acid identity) to a partial G protein-coupled receptor fragment in the Synaptic Pharmaceutical Corporation in-house database, designated PLC29b. PLC29b, which includes part of the amino terminus through TMIII, was originally isolated from a human genomic library using oligonucleotide probes for NPY4. Subsequent screening of a human hippocampal cDNA library yielded an overlapping sequence extending into TMIV. Based on sequence similarity, this human sequence appears to be a partial clone of the human homolog of SNORF2. Additional details can be found in PCT International Publication No. WO 00/18438, the disclosure of which is hereby incorporated by reference in its entirety into this application.

Isolation of the Full-Length Human SNORF2 Receptor Gene (Human NPFF1)

The full-length, intronless version of the human NPFF1 receptor gene may be isolated using standard molecular biology techniques and approaches such as those briefly described below:

Approach #1: To obtain a full-length human NPFF1 receptor, a human cosmid library was screened with a ₃₂P-labeled oligonucleotide probe, BB609, corresponding to the ⅔ loop of the PLC29b clone. A positive clone was isolated and partially sequenced, revealing part of the amino terminus and TMs I and II.

The full-length sequence may be obtained by sequencing this cosmid clone with additional sequencing primers. Since at least two introns are present in this gene, one in the amino terminus and one just after the third transmembrane domain, the full-length intronless gene may be obtained from cDNA using standard molecular biology techniques. For example, a forward PCR primer designed in the 5′UT and a reverse PCR primer designed in the 3′UT may be used to amplify a full-length, intronless gene from cDNA. RT-PCR localization has identified several human tissues which could be used for this purpose, including cerebellum, spinal cord, hippocampus, lung and kidney. Standard molecular biology techniques could be used to subclone this gene into a mammalian expression vector.

Approach #2: Standard molecular biology techniques could be used to screen commercial human cDNA phage libraries by hybridization under high stringency with a ³²P-labeled oligonucleotide probe, BB609, corresponding to the ⅔ loop of the PLC29b clone. One may isolate a full-length human NPFF1 by obtaining a plaque purified clone from the lambda libraries and then subjecting the clone to direct DNA sequencing using primers from the PLC29b sequence. Alternatively, standard molecular biology techniques could be used to screen in-house human cDNA plasmid libraries by PCR amplification of library pools using primers to the human NPFF1 sequence (BB629, forward primer in TMI, and A71, reverse primer in TMIV). A full-length clone could be isolated by Southern hybridization of colony lifts of positive pools with a ³²P_labeled oligonucleotide probe, BB609, corresponding to the ⅔ loop of the PLC29b clone.

Approach #3: As yet another alternative method, one could utilize 3′ and 5′ RACE to generate PCR products from human cDNA expressing human NPFF1 (for example, cerebellum, spinal cord, hippocampus, lung and kidney), which contain the additional sequences of human NPFF1. For 5′ RACE, a reverse primer derived from PLC29b between the amino terminus and TM IV could be used to amplify the additional amino terminus sequence for hNPFF1. For 3′ RACE, a forward primer derived from PLC29b between the amino terminus and TM IV could be used to amplify the additional 3′ sequence for hNPFF1, including TMs 5-7 and the COOH terminus. These RACE PCR product could then be sequenced to determine the missing sequence. This new sequence could then be used to design a forward PCR primer in the 5′UT and a reverse primer in the 3′UT. These primers could then be used to amplify a full-length hNPFF1 clone from human cDNA sources known to express NPFF1 (for example, cerebellum, spinal cord, hippocampus, lung and kidney). Additional details can be found in PCT International Publication No. WO 00/18438, the disclosure of which is hereby incorporated by reference in its entirety into this application.

Cloning of Human NPFF1 Receptor

The sequence of the human NPFF1 (hNPFF1) receptor from the initiating methionine to TMIV was determined to be present in a partial clone, plc29b, found in a Synaptic Pharmaceutical Corporation in-house database. In order to isolate the full-length hNPFF1 receptor cDNA, a human cosmid library (Stratagene) was screened with a ³²P-labeled probe (BB609) corresponding to the II/III loop of plc29b. Partial DNA sequencing of one positive clone from this library, COS28a revealed similar sequence as had been previously shown for plc29b, with an intron downstream of TMIII. In order to obtain sequence in the 3′ end of hNPFF1, COS28a was amplified with a vector primer and BB702, BB703 or BB704, forward primers in TMIV. DNA sequencing of these PCR products resulted in the identification of TMIV through the stop codon.

Next, an in-house human spinal cord library was screened by PCR using a forward primer in the region of the initiating methionine (BB729) and a reverse primer corresponding to TMIV (BB728). One positive pool, W4, was subdivided and a positive sub-pool was screened by colony hybridization with a ³²P-labeled probe from TMI1, BB676. Plasmid DNA was isolated for clone W4-18-4, renamed B098, and DNA sequencing revealed that it was full-length but in the wrong orientation for expression in the expression vector pEXJ. To obtain a full-length hNPFF1 construct in the correct orientation, B098 was amplified with BB757, a forward primer at the initiating methionine which contained an upstream BamHI site, and BB758, a reverse primer at the stop codon which contained a EcoRI site. The products from 3 independent PCR reactions were ligated into pcDNA3.1+ and transformed into DH5α cells. The sequence of one of these transformants, 3.3, was identical to the hNPFF1 sequence previously determined from the consensus of BO98, COS28a and plc29b. Clone 3.3 was renamed B0102.

The hNPFF1 clone contains an open reading frame with 1293 nucleotides and predicts a protein of 430 amino acids. Hydrophobicity analysis reveals seven hydrophobic domains which are presumed to be transmembrane domains. The sequence of hNPFF1 was determined to be most similar to the rat NPFF1 (86% nucleotide identity, 87% amino acid identity) and human NPFF2 (56% nucleotide identity, 49% amino acid identity. The human NPFF1 receptor also shares homology with human orexin₁ (53% nucleotide identity, 35% amino acid identity), human orexin₂ (43% nucleotide identity, 33% amino acid identity), human NPY₂ (47% nucleotide identity, 31% amino acid identity), human CCKA (46% nucleotide identity, 32% amino acid identity), and human CCKB (46% nucleotide identity, 26% amino acid identity). Additional details can be found in PCT International Publication No. WO 00/18438, the disclosure of which is hereby incorporated by reference in its entirety into this application.

Cloning of Human NPFF2 Receptor

Discovery of an Expressed Sequence Tag (EST) AA449919 in GENEMBL Homologous to rNPFF1 (hNPFF2)

A FASTA search of GENEMBL with the full-length sequence of rat NPFF1 (rNPFF1) resulted in the identification of an EST (Accession number AA449919) with a high degree of homology to NPFF1 (57% identity at the DNA level). AA449919 is a 532 bp sequence annotated in Genbank as “Soares total fetus Nb2HF8 9w Homo sapiens cDNA clone 788698 5′ similar to SW:NYR_DROME P25931 NEUROPEPTIDE Y RECEPTOR,” which when translated corresponds to the region between the first extracellular loop and the beginning of the sixth transmembrane domain of rNPFF1. GAP analysis of AA449919 with rNPFF1 indicated that there is 57% DNA identity and a 50% amino acid identity between the two receptor sequences over this region. AA449919 displays 60% DNA identity and 59% amino acid identity over the region that overlaps with the known sequence for hNPFF1 (first extracellular loop to TM4), while over the same range rNPFF1 is 62% and 61% identical to AA449919 at the DNA and amino acid levels, respectively. In comparison, hNPFF1 and rNPFF1 share 86% DNA identity and 92% amino acid identity over this region. Given the strong degree of identity between AA449919 and rNPFF1, AA449919 was given the name NPFF-like (hNPFF2).

Cloning the Full-Length Sequence of (NPFF-like) hNPFF2

To determine the full-length coding sequence of AA449919, 5′/3′ Rapid Amplification of cDNA ends (RACE) was performed on Clontech Human Spleen Marathon-Ready cDNA (Clontech, Palo Alto, Calif.). For 5′ RACE, 5 μl template (human spleen Marathon-Ready cDNA was amplified with oligonucleotide primers JAB256 and AP1, the Expand Long DNA Template PCR System (Boehringer-Mannheim, Indianapolis, Ind.) and the following PCR protocol were used: 94° C. hold for 3 minutes; 5 cycles of 94° C. for 30 seconds, 72° C. for 4 minutes; 5 cycles of 94° C. for 30 seconds, 70° C. for 4 minutes; 30 cycles of 94° C. for 30 seconds, 68° C. for 4 minutes; 68° C. hold for 4 minutes; 4° C. hold until products were ready to be loaded on a gel. 1 μl of this reaction was subjected to a second round of amplification with primers JAB260 and AP2 and the same PCR protocol. For 3′ RACE, 5 μl human spleen Marathon-Ready cDNA was subjected to PCR with primers JAB257 and API with the same PCR protocol that was used for 5′ RACE. 1 μl of this reaction was subjected to another round of amplification using AP2 and JAB258 and the same PCR conditions.

The products were run on a 1% agarose TAE gel and bands greater than 500 bp were extracted from the gel using the QIAQUICK gel extraction kit (QIAGEN, Chatsworth, Calif.). 5 μl of each purified band from the 5′ and 3′ RACE reactions were directly sequenced with primers JAB261 (5′ products) and JAB259 (3′ products) using the ABI Big Dye cycle sequencing protocol and ABI377 sequencers (ABI, Foster City, Calif.). The Wisconsin Package (GCG, Genetics Computer Group, Madison, Wis.) and Sequencer 3.0 (Gene Codes Corporation, Ann Arbor, Mich.) were used to put together the full-length contiguous sequence of hNPFF2 from the AA449919 EST and the RACE products.

To attain the full-length hNPFF-like (hNPFF2) coding sequence for expression, human spinal cord cDNA was amplified in eight independent PCR reactions using the Expand Long Template PCR System with buffer I (four of the eight reactions) or buffer 3 (4 reactions) and two oligonucleotide primers with restriction sites incorporated into their 5′ ends: BB675 is a forward primer upstream of the initiating methionine and contains a BamHI site, and BB663. The PCR conditions for this reaction were as follows: 94° C. hold for 5 minutes; 37 cycles of 94° C. for 30 seconds, 64° C. for 30 seconds, 68° C. for 2 minutes; a 7 minute hold at 68° C., and a 4° C. hold until products were ready to be loaded on a gel. The products were electrophoresed on a 1% agarose TAE gel, and a band of approximately 1.35 kb was cut and purified using the QIAQUICK gel extraction kit. The purified bands of seven of the eight reactions were cut with BamHI and EcoRI, gel purified again using the same method, and ligated into pcDNA3.1(+) (Invitrogen, Carlsbad, Calif.). Eighteen colonies from the subsequent transformations were picked and determined to be positive for NPFF-like by PCR. Eight of these 18 clones were fully sequenced, and one of these, B089, was determined to be a full length clone with no point mutations. This construct was renamed pcDNA3.1-hNPFF2b.

For expression of NPFF-like in oocytes, one ul of each of these eight ligations of the BB675-BB663 PCR product into pcDNA3.1(+) was subjected to PCR with AN35, a pcDNA3.1 primer at the CMV promoter site, and the 3′ NPFF-like primer BB663 using the Expand Long Template PCR System and the following PCR protocol: 94° C. hold for 3 minutes; 37 cycles of 94° C. for 30 seconds, 65° C. for 30 seconds, 68° C. for 2 minutes; a 7 minute hold at 68° C., and a 4° C. hold until products were ready for in vitro transcription. Of the seven PCR reactions, six yielded products of the expected size.

For expression of NPFF2, mRNA transcripts were generated as described for NPFF1, using PCR products from ligation reactions or linearized DNA from B089 as DNA templates. Oocytes were injected with 5-50 ng NPFF2 mRNA and incubated as previously described.

Additional details can be found in PCT International Publication No. WO 00/18438, the disclosure of which is hereby incorporated by reference in its entirety into this application.

Isolation of the Rat Homoloque of NPFF2

To obtain a fragment of the rat homologue of NPFF2, rat genomic DNA (Clontech, Palo Alto, Calif.), rat hypothalamic cDNA or rat spinal cord cDNA was amplified with a forward PCR primer corresponding to TMIV of human NPFF2 (JAB307) and a reverse primer corresponding to TMVI of human NPFF2 (JAB 306). PCR was performed with the Expand Long Template PCR System (Roche Molecular Biochemicals, Indianapolis, Ind.) under the following conditions: 1 minute at 94° C., 2 minutes at 50° C., 2 minutes at 68° C. for 40 cycles, with a pre- and post-incubation of 3 minutes at 94° C. and 4 minutes at 68° C. respectively. Bands of 368 bp from 3 independent PCR reactions were isolated from a TAE gel, purified using the QIAQUICK gel extraction kit (QIAGEN, Chatsworth, Calif.), and sequenced on both strands as described above. The sequences of these 3 PCR products were identical.

To obtain additional sequence for rat NPFF2, reduced stringency PCR was performed using primers designed against the human NPFF2 NH₂ and COOH termini along with PCR primers designed against the rat NPFF2 fragment. For the NH₂ terminal sequence, PCR was performed on rat spinal cord cDNA with BB665, a sense primer just upstream of TMI in human NPFF2, and BB795, an antisense primer in the second extracellular loop of the rat NPFF2. For the COOH terminal sequence, PCR was performed on rat spinal cord cDNA with BB793, a sense primer from the third intracellular loop in rat NPFF2, and BB668, an antisense primer just downstream from TMVII in human NPFF2. PCR was performed using the Expand Long Template PCR System (Roche Biochemicals, Indianapolis, Ind.) with buffer 2 (NH₂ terminal) or buffer 1 (COOH terminal) and the following conditions: 30 seconds at 94° C., 30 seconds at 42° C. (NH₂ terminal) or 50° C. (COOH terminal), 1.5 minutes at 68° C. for 40 cycles, with a pre- and post-incubation of 3 minutes at 94° C. and 4 minutes at 68° C. respectively. A 500 bp band from the NH₂ terminal PCR and a 300 bp band from the COOH terminal PCR were isolated from a TAE gel, purified using the QIAQUICK gel extraction kit (QIAGEN, Chatsworth, Calif.), and sequenced on both strands as described above.

A rat liver genomic phage library (2.75 million recombinants, Stratagene, LaJolla, Calif.) was screened using a ³²P-labeled oligonucleotide probe, BB712, corresponding to the second extracellular loop and TMV of the rat NPFF2 fragment above. Hybridization of nitrocellulose filter overlays of the plates was performed at high stringency: 42° C. in a solution containing 50% formamide, 5×SSC (1×SSC is 0.15M sodium chloride, 0.015M sodium citrate), 1× Denhardt's solution (0.02% polyvinylpyrrolindone, 0.02% Ficoll, 0.02% bovine serum albumin), 7 mM Tris and 25 μg/ml sonicated salmon sperm DNA. The filters were washed at 55° C. in 0.1×SSC containing 0.1% sodium dodecyl sulfate and exposed at −70° C. to Kodak BioMax MS film in the presence of an intensifying screen.

Three positive signals, rNPFF2-1, rNPFF2-4 and rNPFF2-6 were isolated on a tertiary plating. A 3.5 kb fragment, from a BglII/EcoRI digest of DNA isolated from rNPFF2-6, was identified by Southern blot analysis with BB712, subcloned into pcDNA3.1 (Invitrogen, San Diego, Calif.) and used to transform E. coli DH5a cells (Gibco BRL, Gaithersburg Md.). Plasmid DNA from one transformant was sequenced using an ABI 377 sequencer as described above. Sequencing with HK137, a sense primer from TMV of the rat NPFF2 fragment revealed the sequence for TMVII, the COOH terminus and some 3′UT. Sequencing with HK139, an antisense primer from TMII of rNPFF2, revealed the presence an intron upstream of TMII.

To determine if any of the three positive plaques contained sequence upstream of this intron, DNA from each of the clones were spotted onto nitrocellulose and hybridized with HK140, an oligonucleotide probe corresponding to TMI of the rat NPFF2 fragment. The rNPFF2-1 and rNPFF2-4 clones were positive. A 2.1 kb fragment, from a HindIII digest of DNA isolated from rNPFF2-4, was identified by Southern blot analysis with HK140, subcloned into pcDNA3.1 (Invitrogen, San Diego, Calif.) and used to transform E.coli DH5α cells (Gibco BRL, Gaithersburg Md.). Sequencing of this fragment with HK138, an antisense primer from TMI of rat NPFF2, revealed the NH₂ terminus and 5′UT of the rat NPFF2 receptor.

The full-length NPFF2 was amplified from rat spinal cord cDNA using a sense primer in the 5′UT (HK146, also incorporating a BamHI restriction site) and an antisense primer from the 3′UT (HK147, also incorporating a BstXI restriction site) and the Expand Long Template PCR System (Roche Molecular Biochemicals, Indianapolis, Ind.) using buffer 2 and the following PCR conditions: 30 seconds at 94° C., 2.5 minutes at 68° C. for 32 cycles, with a pre- and post-incubation of 5 minutes at 94° C. and 7 minutes at 68° C., respectively. Products from 5 independent PCR reactions were gel-purified. 1 μl of each reaction was used as a template to re-amplify the product using the same PCR conditions. The products were digested with BamHI and BstXI and ligated into a modified pcDNA3.1 vector (Invitrogen, San Diego, Calif.). Products from each PCR reaction were sequenced as above. While a consensus amino acid sequence was determined among the PCR products, there was some ambiguity in the nucleotide sequence at 4 positions. To determine if this represented PCR-induced errors or allelic variations, the areas in question were amplified from several lots of genomic DNA. Sequencing of these genomic products revealed the same ambiguities, suggesting allelic variations at these residues. One construct, K031, was renamed B0119 and later renamed pcDNA3.1-rNPFF2-f. Additional details can be found in PCT International Publication No. WO 00/18438, the disclosure of which is hereby incorporated by reference in its entirety into this application.

Cell Culture

COS-7 cells are grown on 150 mm plates in DMEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of COS-7 cells are trypsinized and split 1:6 every 3-4 days.

Human embryonic kidney 293 cells (HEK-293 cells) are grown on 150 mm plates in DMEM with supplements (10% bovine calf serum, 4 mM glutamine, 100 units/ml penicillin/100 μg/ml streptomycin) at 37° C., 5% CO. Stock plates of 293 cells are trypsinized and split 1:6 every 3-4 days.

Mouse fibroblast LM(tk−) cells are grown on 150 mm plates in D-MEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of LM(tk−) cells are trypsinized and split 1:10 every 3-4 days.

Chinese hamster ovary (CHO) cells were grown on 150 mm plates in HAM's F-12 medium with supplements (10% bovine calf serum, 4 mM L-glutamine and 100 units/ml penicillin/100 ug/ml streptomycin) at 37° C., 5% CO₂. Stock plates of CHO cells are trypsinized and split 1:8 every 3-4 days.

Mouse embryonic fibroblast N1H-3T3 cells are grown on 150 mm plates in Dulbecco's Modified Eagle Medium (DMEM) with supplements (10% bovine calf serum, 4 mM glutamine, 100 units/ml penicillin/100 μg/ml streptomycin) at 37° C., 5% CO₂. Stock plates of NIH-3T3 cells are trypsinized and split 1:15 every 3-4 days.

Sf9 and Sf21 cells are grown in monolayers on 150 mm tissue culture dishes in TMN-FH media supplemented with 10% fetal calf serum, at 27° C., no CO₂. High Five insect cells are grown on 150 mm tissue culture dishes in Ex-Cell 400™ medium supplemented with L-Glutamine, also at 27° C., no CO₂.

Transient Transfection

Receptors studied may be transiently transfected into COS-7 cells by the DEAE-dextran method using 1 μg of DNA/10⁶ cells (Cullen, 1987). In addition, Schneider 2 Drosophila cells may be cotransfected with vectors containing the receptor gene under control of a promoter which is active in insect cells, and a selectable resistance gene, eg., the G418 resistant neomycin gene, for expression of the polypeptides disclosed herein.

Stable Transfection

DNA encoding the human receptors disclosed herein may be co-transfected with a G-418 resistant gene into the human embryonic kidney 293 cell line by a calcium phosphate transfection method (Cullen, 1987). Stably transfected cells are selected with G-418.

Expression of Receptors in Xenopus Oocytes

Expression of genes in Xenopus oocytes is well known in the art (Coleman, Transcription and Translation: A Practical Approach (B. D. Hanes, S. J. Higgins, eds., pp 271-302, IRL Press, Oxford, 1984; Y. Masu, et al. (1987) Nature 329:836-838; Menke, J. G. et al. (1984) J. Biol. Chem. 269(34):21583-21586) and is performed using microinjection into Xenopus oocytes of native mRNA or in vitro synthesized mRNA. The preparation of in vitro synthesized mRNA can be performed using various standard techniques (J. Sambrook et al., Molecular Cloning: A Laboratory Manual, Second Editions, Cold Spring Harbor Laboratory, Cold Spring Harbor, N.Y., 1989) including using T7 polymerase with the mCAP RNA capping kit (Stratagene).

Membrane Preparations

LM(tk−) cells stably transfected with the DNA encoding the human receptor disclosed herein may be routinely converted from an adherent monolayer to a viable suspension. Adherent cells are harvested with trypsin at the point of confluence, resuspended in a minimal volume of complete DMEM for a cell count, and further diluted to a concentration of 10⁶ cells/ml in suspension media (10% bovine calf serum, 10% 10×Medium 199 (Gibco), 9 mM NaHCO₃, 25 mM glucose, 2 mM L-glutamine, 100 units/ml penicillin/100 μg/ml streptomycin, and 0.05% methyl cellulose). Cell suspensions are maintained in a shaking incubator at 37° C., 5% CO₂ for 24 hours. Membranes harvested from cells grown in this manner may be stored as large, uniform batches in liquid nitrogen. Alternatively, cells may be returned to adherent cell culture in complete DMEM by distribution into 96-well microtiter plates coated with poly-D-lysine (0.01 mg/ml) followed by incubation at 37° C., 5% CO₂ for 24 hours.

Generation of Baculovirus

The coding region of DNA encoding the human receptors disclosed herein may be subcloned into pBlueBacIII into existing restriction sites or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined by Pharmingen (in “Baculovirus Expression Vector System: Procedures and Methods Manual”). The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

Radioligand Binding Assays

Cells may be screened for the presence of endogenous human receptor using radioligand binding or functional assays. Cells with either no or a low level of the endogenous human receptors disclosed herein present may be transfected with the human receptors.

Transfected cells from culture flasks are scraped into 5 ml of 20 mM Tris-HCl, 5 mM EDTA, pH 7.5, and lysed by sonication. The cell lysates are centrifuged at 1000 rpm for 5 min. at 4° C., and the supernatant is centrifuged at 30,000×g for 20 min. at 4° C. The pellet is suspended in binding buffer (50 mM Tris-HCl, 60 mM NaCl, 1 mM MgCl, 33 μM EDTA, 33 μM EGTA at pH 7.4 supplemented with 0.2% BSA, 2 μg/ml aprotinin, and 20 μM bestatin). Optimal membrane suspension dilutions, defined as the protein concentration required to bind less than 10% of the added radioligand, are added to 96-well polpropylene microtiter plates containing ³H-labeled compound, unlabeled compounds, and binding buffer to a final volume of 250 μl. In equilibrium saturation binding assays membrane preparations are incubated in the presence of increasing concentrations of [³H]-labeled compound.

The binding affinities of the different compounds are determined in equilibrium competition binding assays, using [¹²⁵I]-labeled compound in the presence of ten to twelve different concentrations of the displacing ligands. Competition assay: 50 pM radioligand, 10-12 points. Binding reaction mixtures are incubated for 2 hr at 25° C., and the reaction stopped by filtration through a double layer of GF filters treated with 0.1% polyethyleneimine, using a cell harvester. Wash buffer: 50 mM Tris-HCl, 0.1% BSA. Radioactivity may be measured by scintillation counting and data are analyzed by a computerized non-linear regression program. Non-specific binding is defined as the amount of radioactivity remaining after incubation of membrane protein in the presence of 1 μM final concentration unlabeled. Protein concentration may be measured by the Bradford method using Bio-Rad Reagent, with bovine serum albumin as a standard.

ATCC Deposits

Plasmids encoding the NPFF receptors have been deposited with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure. These plasmids comprise regulatory elements necessary for expression of DNA in a cell operatively linked to DNA encoding the NPFF receptor so as to permit expression thereof. Plasmids pEXJ-rNPFF1 and pWE15-hNPFF1 were deposited on Sep. 9, 1998, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and were accorded ATCC Accession Nos. 203184 and 203183, respectively. Plasmid pcDNA3.1-hNPFF2b was deposited on Sep. 22, 1998, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Accession No. 203255. Plasmid pcDNA3.1-hNPFF1 was deposited on Jan. 21, 1999, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Accession No. 203605. Plasmid pcDNA3.1-rNPFF2-f was deposited on Aug. 17, 1999, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Procedure and was accorded ATCC Patent Deposit Designation No. PTA-535.

The evidence presented in this invention suggests that compounds that bind to NPFP receptors may be used for the treatment of pain, lower urinary tract disorders, obesity, as well as other indications. The design of such compounds can be optimized by determining their binding interactions at the native serotonin (5HT) and norepinephrine (NE) transporters. Additionally, the NPFF compound(s) would optimally not bind at the following receptors due to possible side effects: human α_{1A} adrenergic, human α_{1B} adrenergic, human α_{1D} adrenergic, human α_{2A} adrenergic, human α_{2B} adrenergic, and human α_{2C} adrenergic receptors; human neuropeptide Y (NPY) Y1, Y2, Y4, and Y5 receptors; and the N-methyl-D-aspartate (NMDA) receptor channel complex.

The binding properties of compounds at different receptors were determined using cultured cell lines that selectively express the receptor of interest. Cell lines were prepared by transfecting the cloned cDNA or cloned genomic DNA or constructs containing both genomic DNA and cDNA encoding the receptors. The methods to obtain the cDNA of the receptors, express said receptors in heterologous systems, and carry out assays to determine binding affinity are described herein below. Furthermore, the binding interactions of compounds at different transporters were determined using tissue preparations and specific assays as described herein below.

α₁ Human Adrenergic Receptors: To determine the binding of compounds to human α₁ receptors, LM(tk−) cell lines stably transfected with the genes encoding the α₁ₐ, α_{1b}, and α_{1d} receptors were used. The nomenclature describing the α₁ receptors was changed recently, such that the receptor formerly designated α₁ₐ is now designated α_{1d}, and the receptor formerly designated α_{1c} is now designated α₁ₐ. The cell lines expressing these receptors were deposited with the ATCC before the nomenclature change and reflect the subtype designations formerly assigned to these receptors. Thus, the cell line expressing the receptor described herein as the α₁ₐ receptor was deposited with the ATCC on Sep. 25, 1992, under ATCC Accession No. CRL 11140 with the designation L-α_{1C}. The cell line expressing receptor described herein as the α_{1d} receptor was deposited with the ATCC on Sep. 25, 1992, under ATCC Accession No. CRL 11138 with the designation L-α_{1A}. The cell line expressing the α_{1b} receptor is designated L-α_{1B}, and was deposited on Sep. 25, 1992, under ATCC Accession No. CRL 11139.

Binding assays using the α_{1A} and α_{1B} adrenergic receptors may be carried out according to the procedures described in U.S. Pat. No. 5,780,485, the disclosure of which is hereby incorporated by reference in its entirety into this application. Binding assays for the human α_{1D} adrenergic receptor may be carried out according to the procedures described in U.S. Pat. No. 6,156,518, the disclosure of which is hereby incorporated by reference in its entirety into this application.

α₂ Human Adrenergic Receptors: To determine the binding of compounds to human α₂ receptors, LM(tk−) cell lines stably transfected with the genes encoding the α_{2A}, α_{2B}, and α_{2C} receptors were used. The cell line expressing the α_{2A} receptor is designated L-α_{2A}, and was deposited on Nov. 6, 1992, under ATCC Accession No. CRL 11180. The cell line expressing the α_{2B} receptor is designated L-NGC-α_{2B}, and was deposited on Oct. 25, 1989, under ATCC Accession No. CRL 10275. The cell line expressing the α_{2C} receptor is designated L-α_{2C}, and was deposited on Nov. 6, 1992, under ATCC Accession No. CRL-11181. Cell lysates were prepared as described herin, and suspended in 25 mM glycyiglycine buffer (pH 7.6 at room temperature). Equilibrium competition binding assay were performed using [⁶H]rauwolscine (0.5 nM), and nonspecific binding was determined by incubation with 10 μM phentolamine. The bound radioligand was separated by filtration through GF/B filters using a cell harvester.

Binding assays using the α₂ adrenergic receptors may be carried out according to the procedures described in U.S. Pat. No. 5,780,485, the disclosure of which is hereby incorporated by reference in its entirety into this application.

Human Histamine H₁ Receptor: The coding sequence of the human histamine H₁ receptor, homologous to the bovine H₁ receptor, is obtained from a human hippocampal cDNA library, and is cloned into the eukaryotic expression vector pCEXV-3. The plasmid DNA for the H₁ receptor is designated pcEXV-H1, and was deposited on Nov. 6, 1992 under ATCC Accession No. 75346. This construct is transfected into COS-7 cells by the DEAE-dextran method. Cells are harvested after 72 hours and lysed by sonication in 5 mM Tris-HCl, 5 mM EDTA, pH 7.5. The cell lysates are centrifuged at 1000 rpm for 5 min at 4° C., and the supernatant is centrifuged at 30,000×g for 20 min. at 4° C. The pellet is suspended in 37.8 mM NaHPO₄, 12.2 mM KH₂PO₄, pH 7.5. The binding of the histamine H₁ antagonist [⁶H]mepyramine (1 nM, specific activity: 24.8 Ci/mM) is done in a final volume of 0.25 ml and incubated at room temperature for 60 min. Nonspecific binding is determined in the presence of 10 μM mepyramine. The bound radioligand is separated by filtration through GF/B filters using a cell harvester.

Human Dopamine D₂ Receptors: The potency of compounds at the D2 receptor is determined using membrane preparations from COS-7 cells transfected with the gene encoding the human D₂ receptor. The coding region for the human D2 receptor is obtained from a human striatum cDNA library, and cloned into the cloning site of PcDNA 1 eukariotic expression vector. The plasmid DNA for the D₂ receptor is designated pcEXV-D2, and was deposited on Nov. 6, 1992 under ATCC Accession No. ATC 75344. This construct is transfected into COS-7 cells by the DEAE-dextran method. Cells are harvested after 72 hours and lysed by sonication in 5 mM Tris-HCl, 5 mM EDTA, pH 7.5. The cell lysates are centrifuged at 1000 rpm for 5 minutes at 4° C., and the supernatant is centrifuged at 30,000×g for 20 minutes at 4° C. The pellet is suspended in 50 mM Tris-HCl (pH 7.4) containing 1 mM EDTA, 5 mM KCl, 1.5 mM CaCl₂, 4 mM MgCl₂, and 0.1% ascorbic acid. The cell lysates are incubated with [3H]spiperone (2 nM), using 10 μM (+)Butaclamol to determine nonspecific binding.

Neuropeptide receptors: Stably transfected cell lines which may be used for binding experiments include, for the Y1 receptor, 293-hY1-5 (deposited Jun. 4, 1996, under ATCC Accession No. CRL-12121); for the Y2 receptor, 293-hY2-10 (deposited Jan. 27, 1994, under ATCC Accession No. CRL-11837); for the Y4 receptor, L-hY4-3 (deposited Jan. 11, 1995, under ATCC Accession No. CRL 11779); and for the Y5 receptor, L-hY5-7 (deposited Nov. 15, 1995, under ATCC Accession No. CRL 11995).

Binding assays using the NPY receptors may be carried out according to the procedures described in U.S. Pat. No. 5,602,024, the disclosure of which is hereby incorporated by reference in its entirety into this application.

NMDA Receptor Channels: The methods to determine binding affinity at native N-methyl-D-aspartate (NMDA) receptor channels are described in Wong E. H. et al. (1988), the disclosure of which is hereby incorporated by reference in its entirety into this application.

Transporters: The binding properties of compounds were evaluated at native, tissue-derived transporters, namely serotonin (5HT) transporter and norepinephrine (NE) transporter, according to protocols described in Owens (1997), the disclosure of which is hereby incorporated by reference in its entirety into this application.

II. Synthesis of Chemical Compounds

Part A. QUINAZOLINO— and QUINOLINO-GUANIDINE Compounds

Compounds described in Part A are labeled with the suffix “A”.

General Methods for Part A:

All reactions were performed under an inert atmosphere (Argon) and the reagents, neat or in appropriate solvents, were transferred to the reaction vessel via syringe and cannula techniques. The parallel synthesis reaction arrays were performed in vials (without an inert atmosphere) using J-KEM heating shakers (Saint Louis, Mo.). Anhydrous solvents (i.e. tetrahydrofuran, toluene and 1-methyl-2-pyrrolidinone) were purchased from Aldrich Chemical Company (Milwaukee, Wis.) and used as received. The compounds described herein were named using ACD/Name program (version 2.51, Advanced Chemistry Development Inc., Toronto, Ontario, M5H2L3, Canada). ¹H and ¹³C spectra were recorded at 300 and 75 MHz (QE-300 Plus by GE, Fremont, Calif.). Chemical shifts are reported in parts per million (ppm) and referenced with respect to the residual (i.e. CHCl₃, CH₃OH) proton of the deuterated solvent. Splitting patterns are designated as s=singlet; d=doublet; t triplet; q=quartet; p=quintet; sextet; septet; br=broad; m=multiplet. Elemental analyses were performed by Robertson Microlit Laboratories, Inc. (Madison, N.J.). Low-resolution electrospray mass spectra (ESMS) were measured and MH⁺ is reported. Thin-layer chromatography (TLC) was carried out on glass plates precoated with silica gel 60 F₂₅₄ (0.25 mm, EM Separations Tech.). Preparative TLC was carried out on glass sheets precoated with silica gel GF (2 mm, Analtech, Newark, Del.). Flash column chromatography was performed on Merck silica gel 60 (230-400 mesh).

The following (Scheme 1) is a representative synthetic scheme for the synthesis of quinazolino-guanidines (Brown 1964, Cowan 1986, Hamann 1998).

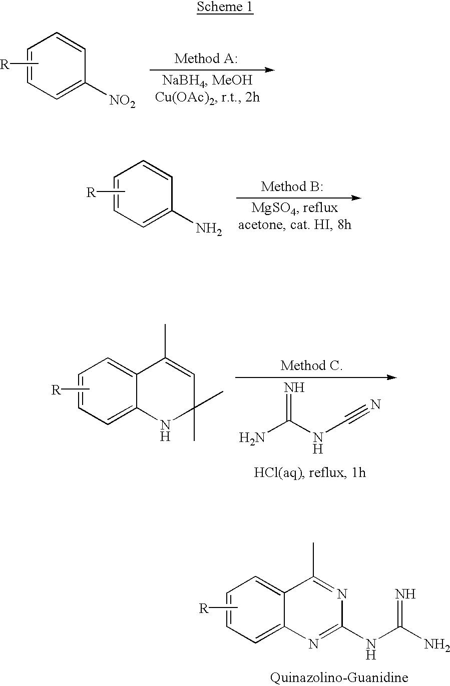

An alternative route (Hynes and Campbell 1997) for the synthesis of quinazolino-guanidines is illustrated below (Scheme 2).

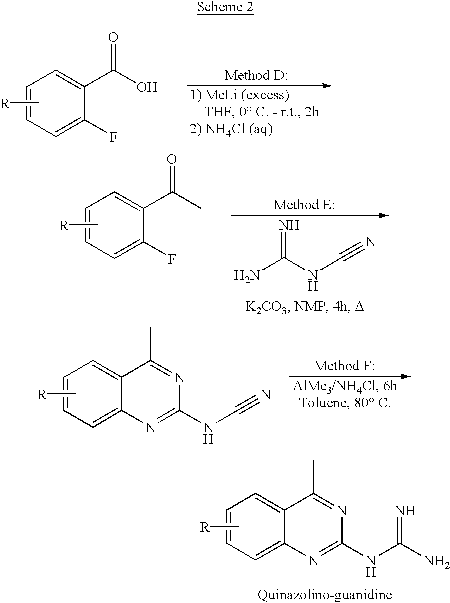

The following (Scheme 3) is a representative synthetic scheme for the synthesis of quinolino-guanidines (Kuhia et al. 1986).

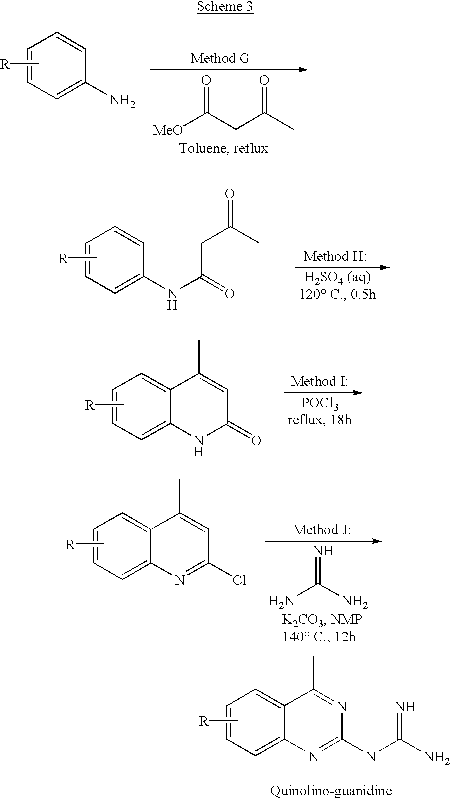

### EXAMPLE 1

The following is a representative example of Methods A-C in Scheme 1 for the synthesis of N-(6,7-dibutoxy-4-methyl-2-quinazolinyl)guanidine (Compound 1018A).

Method A (Yang et al. 1985):

In a flask equipped with a magnetic stirrer, 1,2-dibutoxy-4-nitrobenzene (500 mg, 1.87 mmol) was dissolved in methyl alcohol (23 mL). To this stirring solution was added a saturated aqueous solution of copper (II) acetate (7.5 mL) followed by sodium borohydride (779 mg, 20.6 mmol) added in several small portions so as keep the reaction solution from bumping. After all the sodium borohydride had been added, the solution was allowed to stir at room temperature (r.t.) for an additional 2 h. Brine (100 mL) was added followed by extraction of the aqueous phase with ethyl ether (2×) in a separatory funnel. The combined ethereal extracts were washed with saturated aqueous sodium bicarbonate. The ether was evaporated and the crude material further purified by silica column chromatography eluting with 50% ethyl acetate in hexane (Rf=0.20). The fractions were combined and solvent evaporated to afford 323 mg (73% yield) of 3,4-dibutoxyaniline.

Method B (Vilim and Ziff 1995):

In a flask equipped with a magnetic stirrer, 3,4-dibutoxyaniline (323 mg, 1.36 mmol) was dissolved in acetone (2.3 mL). To this stirring solution was added magnesium sulfate (5.0 eq, 819 mg, 6.80 mmol), tert-butylcatechol (0.03 eq, 7 mg, 0.04 mmol) and iodine (0.05 eq, 17 mg, 0.07 mmol), in that order. The solution was refluxed for 8 h. Upon cooling to r.t., the solution was filtered and the residue further washed with methyl alcohol. The residue was purified by silica column chromatography eluting with 25% ethyl acetate in hexane to afford 230 mg (53% yield) of 6,7-dibutoxy-2,2,4-trimethyl-1,2-dihydroquinoline.

Method C:

In a flask equipped with a magnetic stirrer, 6,7-dibutoxy-2,2,4-trimethyl-1,2-dihydroquinoline (230 mg, 0.72 mmol) was dissolved in 0.5 mL of a solution made up of 0.1 mL of 37% aqueous hydrochloric acid+0.4 mL of water. This solution was refluxed for 1 h. Upon cooling to r.t., 1.5 mL of a 2.0 M ammonia solution in methyl alcohol was added followed by evaporation of the solvent. Purification via preparative TLC eluting with 25% methyl alcohol (containing 2.0 M of ammonia) in chloroform afforded, after isolation of the desired spots (Rf=0.2), 63 mg (25% yield) of N-(6,7-dibutoxy-4-methyl-2-quinazolinyl)guanidine.

Name: 6,7-dibutoxy-2,2,4-trimethyl-1,2-dihydroquinoline. (synthesized using Method B (53% yield)).

Data: ESMS 318 (MH⁺); ¹H NMR (CDCl₃) δ 6.70 (br s, 1H), 6.07 (br s, 1H), 5.19 (br s, 1H), 3.93 (br s, 4H), 1.94 (br s, 3H), 1.75 (septet, 4H, J=7.8 Hz), 1.48 (septet, 4H, J=7.5 Hz), 1.24 (s, 6H), 0.962 (t, 3H, J=7.2 Hz), 0.958 (t, 3H, J=7.2 Hz).

Compound 1018A (synthesized using Method C (25% yield))

Name: N-(6,7-dibutoxy-4-methyl-2-quinazolinyl)guanidine

Data: ESMS 246 (MH⁺); ¹H NMR (CD₃OD) δ 7.89 (br s, 2H), 7.21 (br s, 1H), 7.16 (br s, 1H), 4.13 (t, 2H, J=6.3 Hz), 4.08 (t, 2H, J=6.3 Hz), 2.76 (br s, 3H), 1.88-1.80 (m, 4H), 1.56 (septet, 4H, J=7.5 Hz), 1.013 (t, 3H, J=7.5 Hz), 1.008 (t, 3H, J=7.2 Hz).

### EXAMPLE 2

The following is a representative example of Methods D-F in Scheme 2 for the synthesis of N-(4-methyl-2-quinazolinyl)guanidine (Compound 1001A).

Method D:

In a flask equipped with a magnetic stirrer, a solution of 6-bromo-2-fluorobenzoic acid (1.00 g, 4.57 mmol) dissolved in anhydrous ethyl ether (7 mL) was cooled to −78° C. using a dry ice-acetone bath. Methyl lithium was then added dropwise (6.8 mL of a 1.4 M solution in ethyl ether, 9.59 mmol). The reaction was further stirred at −78° C. for 5 min followed by warming to r.t. by removing the dry ice-acetone bath. After stirring for an additional 30 min at r.t., the solution was poured into a mixture of ice and saturated aqueous solution of ammonium chloride. The aqueous phase was extracted with ethyl ether twice and the combined ethereal extracts washed with brine. The organic phase was dried with anhydrous sodium sulfate, filtered and solvent evaporated. Purification by silica column chromatography eluting with 5% ethyl acetate in hexane (Rf 0.4) afforded 194 mg (20% yield) of 1-(5-bromo-2-fluorophenyl)ethanone.

Method E:

In a flask equipped with a magnetic stirrer, 1-(5-bromo-2-fluorophenyl)ethanone (517 mg, 2.36 mmol) was dissolved in 1-methyl-2-pyrrolidinone (NMP) (3.4 mL). Dicyandiamide (2.0 eq, 397 mg, 4.72 mmol) and potassium carbonate (1.0 eq, 326 mg, 2.36 mmol) were added to the solution and the reaction was heated at 120° C. for 4 h. Upon cooling the reaction to r.t., the solution was filtered and the residue extracted further with methyl alcohol. The methyl alcohol was evaporated. The NMP solution was placed directly on a silica column eluting with 20% methyl alcohol (containing 2.0 M ammonia) in chloroform. Fractions containing the product (Rf=0.5 with 5% methyl alcohol in ethyl acetate) were combined and solvent evaporated to afford 109 mg (18% yield) of 6-bromo-4-methyl-2-quinazolinylcyanamide.

Method F:

To a suspension of ammonium chloride (53.5 mg, 1 mmol) in toluene (1 mL) at r.t. was added 0.5 mL of a 2.0 M trimethylaluminum chloride suspended in toluene (1 mmol). The resulting suspension was stirred at r.t. for 2 h followed by the addition of 4-methyl-2-quinazolinylcyanamide (30 mg, 0.16 mmol). The mixture was heated at 80° C. for 6 h. The reaction mixture was cooled and then poured into a slurry of silica gel in chloroform. The suspension was stirred for 5 min and then filtered. The residue was further washed with methyl alcohol. Purification by preparative TLC eluting with 20% methyl alcohol (containing 2.0 M ammonia) in chloroform (Rf=0.1) afforded N-(4-methyl-2-quinazolinyl)guanidine (11 mg, 34% yield) after isolation of the product.

Compound 1001A

Data: ESMS 202 (MH⁺); ¹H NMR (CD₃OD) δ 8.15 (d, J=8.1, Hz, 1H), 7.80-7.90 (m, 2H), 7.52-7.58 (m, 1H), 2.89 (s, 3H).

### EXAMPLE 3

The following is a representative example of Methods G-J in Scheme 3 for the synthesis of N-(6-ethyl-4-methyl-2-quinolinyl)guanidine (Compound 4002A).

Method G:

To a flask equipped with a magnetic stirrer was added 4-ethylaniline (9.75 g, 80.5 mmol), toluene (20 mL) and methyl acetoacetate (9.1 mL, 85.4 mmol). The reaction mixture was heated to reflux using an Dean-Stark apparatus for 1 h, when the amount of methyl alcohol collected in the apparatus ceased to increase. Upon cooling to r.t., the solvent was evaporated using rotary-evaporator. The crude material was purified by silica column chromatography eluting with 10% methyl alcohol (containing 2.0 M ammonia) in chloroform (Rf=0.6) to afford 5.1 g of N-(4-ethylphenyl)-3-oxobutanamide (31% yield).

Method H:

A flask equipped with a magnetic stirrer containing concentrated sulfuric acid (50 mL) was cooled to 0° C. with an ice-bath followed by the cautious addition of water (25 mL). The solution was heated to 80° C. and N-(4-ethylphenyl)-3-oxobutanamide (5.1 g, 24.8 mmol) added. This solution was stirred and heated at 120° C. for 0.5 h. The reaction was then cooled to r.t. and added to a flask containing ice and water (323 mL). Upon standing overnight in water, crystals formed and were collected via filtration. The crystals were dissolved in a minimum amount of methyl alcohol and filtered through a short pad of silica eluting with 10% methyl alcohol (containing 2.0 M of ammonia) in chloroform. Evaporation of the solvent afforded 3.06 g (66% yield) of 6-ethyl-4-methyl-2(1H)-quinolinone.

Method I:

To a flask equipped with a magnetic stirrer were added 6-ethyl-4-methyl-2(1H)-quinolinone (3.06 g, 16.3 mmol) and phosphorus oxychloride (16.3 mL, 16.3 mmol). The mixture was refluxed for 18 h. The solution was cooled to r.t. and poured into ice water (163 mL) and neutralized to pH=7 using 6 N NaOH (aq). The aqueous phase was extracted with methylene chloride (3×). The organic phase was then filtered through a short pad of silica eluting with methylene chloride. Evaporation of the solvent afforded 2.60 g (77% yield) of 2-chloro-6-ethyl-4-methylquinoline.

Method J

To a flask equipped with a magnetic stirrer were added 2-chloro-6-ethyl-4-methylquinoline (2.02 g, 9.81 mmol), 1-methyl-2-pyrrolidinone (41 mL), potassium carbonate (3.12 g, 22.6 mmol) and guanidine hydrochloride (1.12 g, 11.8 mmol). The mixture was heated at 140° C. for 12 h. Upon cooling to r.t., the mixture was filtered and the residue further extracted with methyl alcohol. The filtrates were combined and the solvent evaporated. The crude material was purified by reverse phase HPLC to afford 46 mg (1% yield) of N-(6-ethyl-4-methyl-2-quinolinyl)guanidine as the trifluoroacetate salt.

Name: N-(4-ethylphenyl)-3-oxobutanamide. (synthesized using Method G (31% yield)).

Data: ESMS 206 (MH⁺); ¹H NMR (CD₃OD) δ 7.42 (d, 2H, J=8.4 Hz), 7.13 (d, 2H, J=8.4 Hz), 3.29 (s, 2H), 2.59 (q, 2H, J=7.8 Hz), 2.25 (s, 3H), 1.19 (t, 3H, J=7.5 Hz).

Name: 6-ethyl-4-methyl-2(1H)-quinolinone. (synthesized using Method H (66% yield)).

Data: ESMS 188 (MH⁺); ¹H NMR (CDCl₃) δ 7.55 (s, 1H), 7.50 (d, 1H, J=8.4 Hz), 7.47 (d, 1H, J=8.4 Hz), 6.69 (s, 1H), 2.77 (q, 2H, J=7.8 Hz), 2.59 (s, 3H), 1.30 (t, 3H, J=7.8 Hz).

Name: 2-chloro-6-ethyl-4-methylquinoline (synthesized using Method I (77% yield)).

Data: ESMS 208 & 206 (MH⁺); ¹H NMR (CD₃OD) δ 7.80 (br d, 1H, J=8.7 Hz), 7.63 (dd, 1H, J=8.7, 1.8 Hz), 7.29 (d, 1H, J=0.6 Hz), 2.84 (q, 2H, J=7.5 Hz), 2.66 (d, 3H, J=0.9 Hz), 1.31 (t, 3H, J=7.5 Hz).

Compound 4002A (class: Quinolino-guanidine; synthesized using Method J).

Name: N-(6-ethyl-4-methyl-2-quinolinyl)guanidine.

Data: ESMS 229 (MH⁺); ¹H NMR (CD₃OD) δ 7.77 (br d, 1H, J=8.7 Hz), 7.57 (dd, 1H, J=8.7, 1.8 Hz), 6.90 (d, 1H, J=0.6 Hz), 2.81 (q, 2H, J=7.5 Hz), 2.64 (d, 3H, J=0.6 Hz), 1.30 (t, 3H, J=7.5 Hz).

### EXAMPLE 4

Compound 3001A (Purchased from Tripos (St. Lousis, Mo.)).

Name: N-(4,7-dimethyl-2-quinazolinyl)guanidine.

### EXAMPLE 5

Compound 1007A (class: Quinazolino-guanidine; Purchased from Sigma).

Name: N-(1-methylbenzo[fjquinazolin-3-yl)guanidine.

### EXAMPLE 6

N-(4-methyl-2-quinolinyl)guanidine was made in the same manner as N-(6-ethyl-4-methyl-2-quinolinyl)guanidine (see Example 3) except that 2-chloro-4-methylquinoline was used in place of 2-chloro-6-ethyl-4-methylquinoline.

Compound 6001A (class: Quinolino-guanidine; synthesized using Method J (67% yield))

Name: N-(4-methyl-2-quinolinyl)guanidine.

Data: ESMS 201 (MH⁺); ¹H NMR (CD₃OD) δ 7.86 (d, J=8.1 Hz, 1H), 7.70 (d, J=8.4 Hz, 1H), 7.52-7.59 (m, 1H), 7.32-7.38 (m, 1H), 6.80 (s, 1H), 2.57 (s, 3H); Anal. (C₁₁H₁₂N₄. 0.15 CHCl₃) calcd, C, 61.39; H, 5.61; N, 25.68; Found, C, 61.81; H, 5.40; N, 26.36.

### EXAMPLE 7

N-(4,7-dimethyl-2-quinolinyl)guanidine was made in the same manner as N-(6-ethyl-4-methyl-2-quinolinyl)guanidine (see Example 3) except that 3-methylaniline was used in place of 4-ethylaniline.

Compound 4006A (Class: Quinolino-guanidine; synthesized using Method J (17% yield))

Name: N-(4,7-dimethyl-2-quinolinyl)guanidine.

Data: ESMS 215 (MH⁺); ¹H NMR (CD₃OD) δ 7.89 (d, J=8.5 Hz, 1H), 7.67 (s, 1H), 7.37 (dd, J=8.5, 1.6 Hz, 1H), 6.88 (s, 1H), 2.65 (s, 3H), 2.51 (s, 3H).

### EXAMPLE 8

N-(4-ethyl-7-methyl-2-quinolinyl)guanidine was made in the same manner as N-(6-ethyl-4-methyl-2-quinolinyl)guanidine (see Example 3) except that 3-methylaniline was used in place of 4-ethylaniline and methyl-3-oxopentanoate in place of methyl acetoacetate.

Compound 6003A (class: Quinolino-guanidine; synthesized using Method J (9% yield))

Name: N-(4-ethyl-7-methyl-2-quinolinyl)guanidine.

Data: ESMS 229 (MH⁺); ¹H NMR (CD₃OD) δ 7.92 (d, J=8.6 Hz, 1H), 7.68 (s, 1H), 7.37 (dd, J=8.5, 1.7 Hz, 1H), 6.90 (s, 1H), 3.07 (q, J=7.2 Hz, 2H), 2.51 (s, 3H), 1.36 (t, J=7.5 Hz, 3H).

### EXAMPLE 9

N-(4,8-dimethyl-2-quinolinyl)guanidine was made in the same manner as N-(6-ethyl-4-methyl-2-quinolinyl)guanidine (see Example 3) except that 2-chloro-4,8-dimethylquinoline was used in place of 2-chloro-6-ethyl-4-methylquinoline.

Compound 6002A (class: Quinolino-guanidine; synthesized using Method J (20% yield))

Name: N-(4,8-dimethyl-2-quinolinyl)guanidine.

Data: ESMS 215 (MH⁺); ¹H NMR (CD₃OD) δ 7.84 (d, J=8.1 Hz, 1H), 7.57 (d, J=7.2 Hz, 1H), 7.41 (dd, J 8.1, 7.2 Hz, 1H), 6.94 (d, J=0.6 Hz, 1H), 2.66 (s, 3H), 2.56 (s, 3H).

### EXAMPLE 10

N-(6-chloro-4-methyl-2-qulnolinyl)guanidine was made in the same manner as N-(6-ethyl-4-methyl-2-quinolinyl)guanidine (see Example 3) except that 2,6-dichloro-4-methylquinoline was used in place of 2-chloro-6-ethyl-4-methylquinoline.

Compound 4005A (class: Quinolino-guanidine; synthesized using Method J (42-71% yield)).

Name: N-(6-chloro-4-methyl-2-quinolinyl)guanidine.

Data: ESMS 231 (MH⁺); ¹H NMR (CD₃OD) δ 7.80 (d, J=2.4 Hz, 1H), 7.88 (d, J=8.7 Hz, 1H), 7.66 (dd, J=9.0, 2.4 Hz, 1H), 7.00 (d, J=0.9 Hz, 1H), 2.65 (s, 3H); Anal. (C₁₁H₁₁ClN₄+0.1 CHCl₃. 0.7H₂O) calcd, C, 51.43; H, 4.86; N, 21.61; Found, C, 51.41; H, 4.85; N, 21.78.

### EXAMPLE 11

N-(1-methylbenzo[f]quinolin-3-yl)guanidine was made in the same manner as N-(6-ethyl-4-methyl-2-quinolinyl)guanidine (see Example 3) except that 3-chloro-1-methylbenzo[f]quinoline was used in place of 2-chloro-6-ethyl-4-methylquinoline.

Compound 4009A (class: Quinolino-guanidine; synthesized using Method J (21% yield))

Name: N-(1-methylbenzo[flquinolin-3-yl)guanidine.

Data: ESMS 251 (MH⁺); ¹H NMR (CD₃OD) δ 8.63 (d, J=7.8 Hz, 1H), 7.83-7.87 (m, 2H), 7.46-7.63 (m, 3H), 6.91 (s, 1H), 2.93 (s, 3H).

### EXAMPLE 12

N-(6-methoxy-4-methyl-2-quinolinyl)guanidine was made in the same manner as N-(6-ethyl-4-methyl-2-quinolinyl)guanidine (see Example 3) except that 2-chloro-6-methoxy-4-methylquinoline was used in place of 2-chloro-6-ethyl-4-methylquinoline.

Compound 4004A (class: Quinolino-guanidine; synthesized using Method J (13% yield)).

Name: N-(6-methoxy-4-methyl-2-quinolinyl)guanidine.

Data: ESMS 231 (MH⁺); ¹H NMR (CD₃OD) δ 7.80 (d, J=9.3 Hz, 1H), 7.34 (dd, J=9.0, 2.7 Hz, 1H), 6.98 (d, J=0.9 Hz, 1H), 3.92 (s, 3H), 2.65 (s, 3H).

### EXAMPLE 13

N-(4,5,7-trimethyl-2-quinolinyl)guanidine was made in the same manner as N-(6-ethyl-4-methyl-2-quinolinyl)guanidine (see Example 3) except that 3,5-dimethylaniline was used in place of 4-ethylaniline.

Compound 4008A (class: Quinolino-guanidine; synthesized using Method J (7% yield)).

Name: N-(4,5,7-trimethyl-2-quinolinyl)guanidine.

Data: ESMS 229 (MH⁺); ¹H NMR (CD₃OD) δ 7.51 (s, 1H), 7.13 (s, 1H), 6.80 (s, 1H), 2.85 (s, 3H), 2.82 (s, 3H), 2.42 (s, 3H).

### EXAMPLE 14

N-(4,6-dimethyl-2-quinolinyl)guanidine was made in the same manner as N-(6-ethyl-4-methyl-2-quinolinyl)guanidine (see Example 3) except that 4-methylaniline was used in place of 4-ethylaniline.

Compound 4001A (class: Quinolino-guanidine; synthesized using Method J (5% yield)).

Name: N-(4,6-dimethyl-2-quinolinyl)guanidine.

Data: ESMS 215 (MH⁺); ¹H NMR (CD₃OD) δ 7.79 (dd, J=4.2, 4,2 Hz, 2H), 7.89 (dd, J 8.7, 1.8 Hz, 1H), 7.75 (d, J=0.9 Hz, 1H), 2.67 (d, J=0.9 Hz, 3H), 2.52 (s, 3H).

### EXAMPLE 15

N-(4-methyl-6-phenyl-2-quinolinyl)guanidine was made in the same manner as N-(6-ethyl-4-methyl-2-quinolinyl)guanidine (see Example 3) except that 2-chloro-4-methyl-6-phenylquinoline was used in place of 2-chloro-6-ethyl-4-methylquinoline.

Compound 4003A (class: Quinolino-guanidine; synthesized using Method J (28% yield)).

Name: N-(4-methyl-6-phenyl-2-quinolinyl)guanidine.

Data: ESMS 277 (MH⁺); ¹H NMR (CD₃OD) δ 8.10 (d, J=1.2 Hz, 1H), 7.90-7.98 (m, 2H), 7.65-7.73 (m, 2H), 7.32-7.50 (m, 3H), 7.01 (s, 1H), 2.73 (s, 3H).

### EXAMPLE 16

N-(7-ethyl-4-methyl-2-quinazolinyl)guanidine was made in the same manner as N-(6-ethyl-4-methyl-2-quinolinyl)guanidine (see Example 3) except that 3-ethylaniline was used in place of 4-ethylaniline.

Compound 1020A (class: Quinazolino-guanidine; synthesized using Method C (52% yield)).

Name: N-(7-ethyl-4-methyl-2-quinazolinyl)guanidine.

Data: ESMS 230 (MH⁺); ¹H NMR (CD₃OD) δ 8.09 (d, J=8.4 Hz, 1H), 7.68 (d, J=0.9 Hz, 1H), 7.49 (dd, J=8.4, 1.5 Hz, 1H), 2.88 (s, 3H), 2.86 (q, J=7.6 Hz, 2H), 1.32 (t, J=7.5 Hz, 3H).

### EXAMPLE 17

N-(7-fluoro-4-methyl-2-quinolinyl)guanidine was made in the same manner as N-(6-ethyl-4-methyl-2-quinolinyl)guanidine (see Example 3) except that 3-fluoroaniline was used in place of 4-ethylaniline.

Compound 4007A (class: Quinolino-guanidine; synthesized using Method J (36% yield)).

Name: N-(7-fluoro-4-methyl-2-quinolinyl)guanidine.

Data: ESMS 219 (MH⁺); ¹H NMR (CD₃OD) δ 8.00 (dd, J=9.0, 6.0 Hz, 1H), 7.57 (dd, J=10.2, 2.4 Hz, 1H), 7.30 (dt, J=8.7, 2.7 Hz, 1H), 6.88 (s, 1H), 2.64 (s, 3H); Anal. (C₁₁H₁₁FN₄ 1.1 CF₃CO₂H) calcd, C, 46.13; H, 3.55; N, 1630; Found, C, 46.66; H, 3.31; N, 16.41.

### EXAMPLE 18

Compound 1002A (class: Quinazolino-guanidine).

Name: N-(4,6-dimethyl-2-quinazolinyl)guanidine.

A compound purchased from Tripos was found to have the wrong structure assignment and to contain an impurity. Tripos' incorrect structure assignment was 2-[(4,7-dimethyl-2-quinazolinyl)amino]-4-quinazolinol. By NMR and MS techniques, the sample was determined to be a mixture of N-(4,6-dimethyl-2-quinazolinyl) guanidine and methyl 2-aminobenzoate, which was separated by preparative TLC to afford pure N-(4,6-dimethyl-2-quinazolinyl)guanidine.

Data: ESMS 216 (MH+-NH₃); ¹H NMR (CD₃OD) δ 7.97 (s, 1H), 7.77 (br s, 2H, 2^{nd} Order Coupling), 2.89 (s, 3H), 2.54 (s, 3H); ¹³C NMR (CD₃OD) 172.2, 156.4, 153.4, 147.8, 137.7, 137.6, 127.0, 124.9, 122.1, 21.0, 20.7.

### EXAMPLE 19

N-(6,7-difluoro-4-methyl-2-quinazolinyl)guanidine was made in the same manner as N-(6,7-dibutoxy-4-methyl-2-quinazolinyl)guanidine (see Example 1, steps B and C) except that 3,4-difluoroaniline was used in place of 3,4-dibutoxyaniline.

Compound 1019A (class: Quinolino-guanidine; synthesized using Method J (42% yield)).

Name: N-(6,7-difluoro-4-methyl-2-quinazolinyl)guanidine.

Data: ESMS 238 (MH⁺); ¹H NMR (CD₃OD) δ 7.98 (dd, J=10.8, 8.7 Hz, 1H), 7.59 (dd, J=11.4, 7.5 Hz, 1H), 2.80 (s, 3H); Anal. (C₁₀H₉F₂N₅. 0.21 SiO₂) calcd, C, 48.08; H, 3.631; N, 28.03; Found, C, 47.61; H, 3.61; N, 28.46.

### EXAMPLE 20

N-(7-bromo-4-methyl-2-quinazolinyl)guanidine was made in the same manner as N-(6,7-dibutoxy-4-methyl-2-quinazolinyl)guanidine (see Example 1) except that 3-bromoaniline was used in place of 3,4-dibutoxyaniline.

Name: 7-bromo-2,2,4-trimethyl-1,2-dihydroquinoline (Synthesized using Method B (28%)).

Data: ESMS 254 & 252 (MH⁺); ¹H NMR (CDCl₃) δ 6.88 (d, 1H, J=8.1 Hz), 6.72 (dd, 1H, J=8.1, 2.1 Hz), 6.57 (d, 1H, J=2.1 Hz), 5.31 (br d, 1H, J=1.2 Hz), 1.95 (d, 3H, J=1.5 Hz), 1.27 (s, 6H).

Compound 1014A (class: Quinazolino-guanidine; synthesized using Method C (7% yield)).

Name: N-(7-bromo-4-methyl-2-quinazolinyl)guanidine.

Data: ESMS 282 & 280 (MH⁺); ¹H NMR (CD₃OD) δ 8.08 (d, 1H, 7.8 Hz), 7.88 (s, 1H), 7.69 (br d, 1H, J=8.7 Hz), 2.89 (s, 3H).

### EXAMPLE 21

N-(6-bromo-4-methyl-2-quinazolinyl)guanidine was made in the same manner as N-(6,7-dibutoxy-4-methyl-2-quinazolinyl)guanidine (see Example 1) except that 4-bromoaniline was used in place of 3,4-dibutoxyaniline.

Name: 6-bromo-2,2,4-trimethyl-1,2-dihydroquinoline.

(Synthesized using Method B (22% yield)).

Data: ESMS 254 & 252 (MH⁺); ¹H NMR (CDCl₃) δ 7.12 (d, 1H, J=2.1 Hz), 7.04 (dd, 1H, J=8.4, 2.1 Hz), 6.31 (br d, 1H, J=8.4 Hz), 5.33 (br s, 1H), 1.95 (d, 3H, J=1.5 Hz), 1.26 (s, 6H).

Compound 1026A (class: Quinazolino-guanidine; synthesized using Methods C (4% yield)).

Name: N-(6-bromo-4-methyl-2-quinazolinyl)guanidine.

Data: ESMS 282 & 280 (MH⁺); ¹H NMR (CD₃OD) δ 8.40 (d, 1H, J=2.1 Hz), 8.02 (dd, 1H, J=8.7, 2.1 Hz), 7.85 (d, 1H, J=9.0 Hz), 2.91 (s, 3H).

### EXAMPLE 22

N-[4-methyl-7-(trifluoromethoxy)-2-quinazolinyl]guanidine was made in the same manner as N-(6,7-dibutoxy-4-methyl-2-quinazolinyl)guanidine (see Example 1) except that 3-trifluoromethoxyaniline was used in place of 3,4-dibutoxyaniline.

Name: 2,2,4-trimethyl-7-(trifluoromethoxy)-1,2-dihydroquinoline (Synthesized using Method B (29% yield)).

Data: ESMS 258 (MH⁺); ¹H NMR (CDCl₃) δ 7.00 (d, 1H, J=8.1 Hz), 6.44 (dd, 1H, J=7.5, 1.2 Hz), 6.26 (br s, 1H), 5.30 (d, 1H,J=1.5 Hz), 1.96 (d, 3H, J=1.5 Hz), 1.28 (s, 6H).

Compound 1036A

Name: N-[4-methyl-7-(trifluoromethoxy)-2-quinazolinyl]guanidine (class: Quinazolino-guanidine; synthesized using Method C (5% yield).

Data: ESMS 286 (MH⁺); ¹H NMR (CD₃OD) δ 8.26 (d, 1H, J=9.3 Hz), 7.69 (br s, 1H), 7.39 (dm, 1H, J=7.2 Hz), 2.89 (s, 3H)

### EXAMPLE 23

N-(6-chloro-4-methyl-2-quinazolinyl)guanidine was made in the same manner as N-(6,7-dibutoxy-4-methyl-2-quinazolinyl)guanidine (see Example 1) except that 4-chloroaniline was used in place of 3,4-dibutoxyaniline.

Compound 1013A

Name: N-(6-chloro-4-methyl-2-quinazolinyl)guanidine (class: Quinazolino-guanidine; synthesized using Method C (35% yield)).

Data: ESMS 236 (MH⁺); ¹H NMR (CD₃OD) δ 8.20 (t, J=1.5 Hz, 1H), 7.86 (d, J=1.5 Hz, 2H), 2.89 (s, 3H); Anal. (C₁₁H₁₀ClN₅. 0.21 CHCl₃. 0.7H₂O) calcd, C, 44.86; H, 4.28; N, 25.62; Found, C, 44.62; H, 4.28; N, 25.91.

### EXAMPLE 24

N-(6-methoxy-4-methyl-2-quinazolinyl)guanidine was made in the same manner as N-(6,7-dibutoxy-4-methyl-2-quinazolinyl)guanidine (see Example 1) except that 4-methoxyaniline was used in place of 3,4-dibutoxyaniline.

Compound 1011A (class: Quinazolino-guanidine; synthesized using Method C (13% yield)).

Name: N-(6-methoxy-4-methyl-2-quinazolinyl)guanidine.

Data: ESMS 232 (MH⁺); ¹H NMR (CD₃OD) δ 7.77 (d, J=9.0 Hz, 1H), 7.54 (dd, J=9.3, 2.7 Hz, 1H), 7.38 (d, J=2.7 Hz, 1H), 3.94 (s, 3H), 2.87 (s, 3H).

### EXAMPLE 25

N-(7-isopropyl-4-methyl-2-quinazolinyl)guanidine was made in the same manner as N-(6,7-dibutoxy-4-methyl-2-quinazolinyl)guanidine (see Example 1) except that 3-isopropylaniline was used in place of 3,4-dibutoxyaniline.

Compound 1021A (class: Quinazolino-guanidine; synthesized using Method C (85%), except that reverse phase (C18) column chromatography eluting with acetonitrile was used in place of normal phase).

Name: N-(7-isopropyl-4-methyl-2-quinazolinyl)guanidine.

Data: ESMS 244 (MH⁺); ¹H NMR (CD₃OD) δ 8.11 (d, 1H, J=8.4 Hz), 7.72 (d, 1H, J=1.5 Hz), 7.54 (dd, 1H, J=8.7, 1.8 Hz), 3.12 (septet, 1H, J=6.9 Hz), 2.88 (s, 3H), 1.34 (d, 6H, J=6.9 Hz).

### EXAMPLE 26

N-[4-methyl-6-(trifluoromethoxy)-2-quinazolinyl] guanidine was made in the same manner as N-(6,7-dibutoxy-4-methyl-2-quinazolinyl)guanidine (see Example 1) except that 4-trifluoromethoxyaniline was used in place of 3,4-dibutoxyaniline.

Name: 2,2,4-trimethyl-6-(trifluoromethoxy)-1,2-dihydroquinoline. (Synthesized using Method B (19% yield)).

Data: ESMS 258 (MH⁺); ¹H NMR (CDCl₃) δ 6.89 (br d, 1H, J=1.8 Hz), 6.83 (br dd, 1H, J=8.7, 1.5 Hz), 6.37 (d, 1H, J=8.4 Hz), 5.37 (br s, 1H), 1.96 (d, 3H, J=1.2 Hz), 1.28 (s, 6H).

Compound 1030A (synthesized using Method C (11% yield)).

Name: N-[4-methyl-6-(trifluoromethoxy)-2-quinazolinyl]guanidine.

Data: ESMS 286 (MH⁺); ¹H NMR (CD-OD) δ 8.02 (br d, 1H, J=2.1 Hz), 7.90 (d, 1H, J=9.3 Hz), 7.77 (br dd, 1H, J=8.7, 1.8 Hz), 2.88 (s, 3H).

### EXAMPLE 27

N-(4-methyl-6-pentyl-2-quinazolinyl)guanidine was made in the same manner as N-(6,7-dibutoxy-4-methyl-2-quinazolinyl)guanidine (see Example 1) except that 4-pentylaniline was used in place of 3,4-dibutoxyaniline.

Name: 2,2,4-trimethyl-6-pentyl-1,2-dihydroquinoline (synthesized using Method B (32% yield).

Data: ESMS 244 (MH⁺); ¹H NMR (CDCl₃) δ 6.86 (d, 1H, J=0.9 Hz), 6.80 (dd, 1H, J=7.8, 0.9 Hz), 6.37 (d, 1H, J=7.8 Hz), 5.30 (br s, 1H), 2.47 (t, 2H, J=7.5 Hz), 1.98 (d, 3H, J=0.9 Hz), 1.54 (br p, 2H, J=7.2 Hz), 1.34-1.25 (m, 4H), 1.26 (s, 6H), 0.88 (br t, 3H, J=6.6 Hz).

Compound 2001A

Name: N-(4-methyl-6-pentyl-2-quinazolinyl)guanidine (synthesized using Method C (9-41% yield). crystallization from MeOH and reverse phase (C18) HPLC were required).

Data: ESMS 272 (MH⁺); ¹H NMP (CD₃OD) δ 7.97 (s, 1H, 2^{nd} order coupling), 7.81 (br s, 2H, 2nd order coupling), 2.91 (s, 3H), 2.82 (t, 2H, J=7.8 Hz), 1.73-1.68 (m, 2H), 1.38-1.34 (m, 4H), 0.90 (br t, 3H, J=6.6 Hz).

### EXAMPLE 28

N-(4,6,7-trimethyl-2-quinazolinyl)guanidine was made in the same manner as N-(6,7-dibutoxy-4-methyl-2-quinazolinyl)guanidine (see Example 1) except that 3,4-dimethylaniline was used in place of 3,4-dibutoxyaniline.

Name: 2,2,4,6,7-pentamethyl-1,2-dihydroquinoline (synthesized using Method B (47% yield)).

Data: ¹H NMR (CDCl₃) δ 6.82 (s, 1H), 6.28 (s, 1H), 5.24 (d, 1H, J=0.9 Hz), 2.14 (s, 6H), 1.96 (d, 3H, J=1.2 Hz), 1.24 (s, 6H).

Compound 1015A (class: Quinazolino-guanidine; synthesized using Method C (12% yield)).

Name: N-(4,6,7-trimethyl-2-quinazolinyl)guanidine.

Data: ESMS 230 (MH⁺); ¹H NMR (CD₃OD) δ 7.93 (s, 1H), 7.66 (s, 1H), 2.87 (s, 3H), 2.48 (s, 3H), 2.47 (s, 3H).

### EXAMPLE 29

N-[6-(benzyloxy)-4-methyl-2-quinazolinyl]guanidine was made in the same manner as N-(6,7-dibutoxy-4-methyl-2-quinazolinyl)guanidine (see Example 1) except that 4-benzyloxyaniline was used in place of 3,4-dibutoxyaniline.

Name: 6-(benzyloxy)-2,2,4-trimethyl-1,2-dihydroquinoline (synthesized using Method B (60% yield)).

Data: ESMS 280 (MH⁺).

Compound 1028A (class: Quinazolino-guanidine; synthesized using Method C (6% yield)).

Name: N-[6-(benzyloxy)-4-methyl-2-quinazolinyl]guanidine.

Data: ESMS 308 (MH⁺); ¹H NMR (CD₃OD) δ 7.83 (br d, 1H, J=9.0 Hz), 7.66 (br d, 1H, J=9.0 Hz), 7.55-7.48 (m, 3H), 7.40-4.31 (m, 4H), 5.25 (s, 2H), 2.87 (s, 3H).

### EXAMPLE 30

N-[7-(1-hydroxyethyl)-4-methyl-2-quinazolinyl]guanidine was made in the same manner as N-(6,7-dibutoxy-4-methyl-2-quinazolinyl)guanidine (see Example 1) except that 3-(1-hydroxyethyl)aniline was used in place of 3,4-dibutoxyaniline.

Compound 1035A Name: N-[7-(1-hydroxyethyl)-4-methyl-2-quinazolinyl]guanidine (synthesized using Method C (86% yield)).

Data: ESMS 246 (MH⁺); ¹H NMR (CD₃OD) δ 8.17 (d, 1H, J=8.7 Hz), 7.87 (s, 1H), 7.64 (d, 1H, J=8.7 Hz), 5.02 (q, 1H, J=6.6 Hz), 2.91 (br s, 3H), 1.50 (d, 3H, J=6.6 Hz).

### EXAMPLE 31

N-(6-ethyl-4-methyl-2-quinazolinyl)guanidine was made in the same manner as N-(6,7-dibutoxy-4-methyl-2-quinazolinyl)guanidine (see Example 1) except that 4-ethylaniline was used in place of 3,4-dibutoxyaniline.

Name: 6-ethyl-2,2,4-trimethyl-1,2-dihydroquinoline (synthesized using Method B (38% yield)).

Data: ESMS 202 (MH⁺); ¹H NMR (CDCl₃) δ 6.89 (d, 1H, J=1.5 Hz), 6.83 (dd, 1H, J 8.1, 1.8 Hz), 6.39 (d, 1H, J=8.1 Hz), 5.31 (d, 1H, J=0.9 Hz), 2.52 (q, 2H, J=7.5 Hz), 1.99 (d, 3H, J=12 Hz), 1.26 (s, 6H), 1.19 (t, 3H, J=7.5 Hz).

Compound 1003A (class: Quinazolino-guanidine; synthesized using Method C (7% yield)).

Name: N-(6-ethyl-4-methyl-2-quinazolinyl)guanidine.

Data: ESMS 230 (MH⁺); ¹H NMR (CD₃OD) δ 7.97 (br s, 1H, 2^{nd} order coupling), 7.818 (s, 1H, 2^{nd} order coupling), 7.815 (s, 1H, 2^{nd} order coupling), 2.91 (s, 3H), 2.85 (q, 2H, J=7.5 Hz), 1.32 (t, 3H, J=7.5 Hz).

### EXAMPLE 32

N-(6-sec-butyl-4-methyl-2-quinazolinyl)guanidine was made in the same manner as N-(6,7-dibutoxy-4-methyl-2-quinazolinyl)guanidine (see Example 1) except that 4-sec-butylaniline was used in place of 3,4-dibutoxyaniline.

Name: 6-sec-butyl-2,2,4-trimethyl-1,2-dihydroquinoline (synthesized using Method B (50% yield)).

Data: ESMS 230 (MH⁺); ¹H NMR (CDCl₃) δ 6.86 (br s, 1H), 6.80 (br d, 1H, J=8.7 Hz), 6.39 (br d, 1H, J=8.5 Hz), 5.30 (br s, 1H), 2.50-2.40 (m, 1H), 1.99 (s, 3H), 1.53 (q, 2H, J=7.2 Hz), 1.27 (s, 6H), 1.19 (d, 3H, J=6.9 Hz), 0.82 (t, 3H, J=7.5 Hz).

Compound 2002A (class: Quinazolino-guanidine; synthesized using Method C (36% yield)).

Name: N-(6-sec-butyl-4-methyl-2-quinazolinyl)guanidine.

Data: ESMS 258 (MH⁺); ¹H NMR (CD₃OD) δ 7.90 (s, 1H, 2^{nd} order coupling), 7.787 (s, 1H, 2^{nd} order coupling), 7.791 (s, 1H, 2 d order coupling), 2.88 (s, 3H), 2.83 (septet, 1H, J=7.2 Hz), 1.69 (p, 2H, J=7.2 Hz), 1.31 (d, 3H, J=6.9 Hz), 0.83 (t, 3H, J=7.2 Hz).

### EXAMPLE 33

N-(4-methylfuro[2,3-g]quinazolin-2-yl)guanidine was made in the same manner as N-(6,7-dibutoxy-4-methyl-2-quinazolinyl)guanidine (see Example 1) except that 5-nitro-[2,3]-benzofuran was used in place of 1,2-dibutoxy-4-nitrobenzene.

Name: 6,6,8-trimethyl-5,6-dihydrofuro[2,3-g]quinoline (synthesized using Method B (70% yield)).

Data: ¹H NMR (CDCl₃) δ 7.53 (br s, 1H), 7.21 (dd, 1H, J=8.4, 0.6 Hz), 6.94 (br s, 1H), 6.51 (d, 1H, J=8.4 Hz), 5.38 (d, 1H, J=1.2 Hz), 2.29 (d, 3H, J=1.2 Hz), 1.29 (s, 6H).

Compound 1039A

Name: N-(4-methylfuro[2,3-g]quinazolin-2-yl)guanidine (class: Quinazolino-guanidine; synthesized using Method C (85% yield)).

Data: ESMS 242 (MH⁺); ¹H NMR (CD₃OD) δ 8.18 (d, 1H, J=9.6 Hz), 8.14 (br s, 1H,), 7.85 (d, 1H, J=9.0 Hz), 7.53 (br s, 1H), 3.13 (s, 3H).

### EXAMPLE 34

N-(6-butoxy-4-methyl-2-quinazolinyl)guanidine was made in the same manner as N-(6,7-dibutoxy-4-methyl-2-quinazolinyl)guanidine (see Example 1) except that 4-butoxyaniline was used in place of 3,4-dibutoxyaniline.

Name: butyl 2,2,4-trimethyl-1,2-dihydro-6-quinolinyl ether.

(synthesized using Method B (14% yield)).

Data: ESMS 246 (MH⁺); ¹H NMR (CDCl₃) δ 6.69 (br d, 1H, J=2.7 Hz), 6.60 (dd, 1H, J=8.4, 2.7 Hz), 6.40 (d, 1H, J=8.4 Hz), 5.36 (br s, 1H), 3.89 (t, 2H, J=6.6 Hz), 1.97 (d, 3H, J=0.9 Hz), 1.72 (p, 2H, J=5.7 Hz), 1.47 (septet, 2H, J=7.2 Hz), 1.25 (s, 6H), 0.96 (t, 3H, J=7.2 Hz).

Compound 1012A (class: Quinazolino-guanidine; synthesized using Method C (12% yield)).

Name: N-(6-butoxy-4-methyl-2-quinazolinyl)guanidine.

Data: ESMS 247 (MH⁺); ¹H NMR (CD,OD) δ 7.81 (d, 1H, J=9.0 Hz), 7.56 (dm, 1H, J=9.3 Hz), 7.50-7.40 (m, 1H), 4.14 (t, 2H, J=6.0 Hz), 2.89 (s, 3H), 1.84 (p, 2H, J=7.8 Hz), 1.55 (septet, 2H, J=7.5 Hz), 1.01 (t, 3H, J=7.5 Hz).

### EXAMPLE 35

N-(4-methyl-6-phenoxy-2-quinazolinyl)guanidine was made in the same manner as N-(6,7-dibutoxy-4-methyl-2-quinazolinyl)guanidine (see Example 1) except that 4-phenoxyaniline was used in place of 3,4-dibutoxyaniline.

Name: 2,2,4-trimethyl-6-phenoxy-1,2-dihydroquinoline (synthesized using Method B (10% yield).

Data: ¹H NMR (CDCl₃) δ 7.187 (t, 2H, J=7.8 Hz), 6.91 (t, 1H, J=6.9 Hz), 6.81 (d, 2H, J=7.8 Hz), 6.68 (d, 1H, J=2.1 Hz), 6.60 (dd, 1H, J=8.4, 2.1 Hz), 6.53 (d, 1H, J=8.4 Hz), 5.37 (br s, 1H), 1.88 (d, 3H, J=1.2 Hz), 1.23 (s, 6H).

Compound 1032A (class: Quinazolino-guanidine; synthesized using Method C (11% yield)).

Name: N-(4-methyl-6-phenoxy-2-quinazolinyl)guanidine.

Data: ESMS 294 (MH⁺); ¹H NMR (CD₃OD) δ 7.93 (d, 1H, J=9.0 Hz), 7.66 (dd, 1H, J=9.0, 2.7 Hz), 7.58 (d, 1H, J=2.7 Hz), 7.42 (t, 2H, J=7.5 Hz), 7.20 (t, 1H, J=7.5 Hz), 7.09 (br d, 2H, J=7.5 Hz), 2.79 (s, 3H)

### EXAMPLE 36

N-(6-cyclohexyl-4-methyl-2-quinazolinyl)guanidine was made in the same manner as N-(6,7-dibutoxy-4-methyl-2-quinazolinyl)guanidine (see Example 1) except that 4-cyclohexylaniline was used in place of 3,4-dibutoxyaniline.

Name: 6-cyclohexyl-2,2,4-trimethyl-1,2-dihydroquinoline.

(synthesized using Method B (47% yield).

Data: ¹H NMR (CDCl₃) δ 7.00 (d, 1H, J=1.8 Hz), 6.94 (dd, 1H, J=8.1, 1.8 Hz), 6.45 (3, 1H, J=8.1 Hz), 5.38 (d, 1H, J=1.2 Hz), 2.55-2.42 (m 1H), 2.09 (s, 3H), 1.97-1.91 (m, 5H), 1.83 (br d, 1H, J=12 Hz), 1.55-1.42 (m, 4H), 1.34 (s, 6H).

Compound 1029A (class: Quinazolino-guanidine; synthesized using Method C (14% yield)).

Name: N-(6-cyclohexyl-4-methyl-2-quinazolinyl)guanidine.

Data: ESMS 284 (MH⁺).

### EXAMPLE 37

N-[4-methyl-6-(pentyloxy)-2-quinazolinyl]guanidine was made in the same manner as N-(6,7-dibutoxy-4-methyl-2-quinazolinyl)guanidine (see Example 1) except that 4-pentyloxyaniline was used in place of 3,4-dibutoxyaniline.

Name: Pentyl 2,2,4-trimethyl-1,2-dihydro-6-quinolinyl ether.

(synthesized using Method B (59% yield)

Data: ESMS 260 (MH⁺).

Compound 1031A (class: Quinazolino-guanidine; synthesized using Method C (13% yield)).

Name: N-[4-methyl-6-(pentyloxy)-2-quinazolinyl]guanidine.

Data: ESMS 288 (MH⁺); ¹H NMR (CD₃OD) δ 7.82 (d, 1H, J=9.3 Hz), 7.57 (dd, 1H, J=9.0, 2.4 Hz), 7.41 (d, 1H, J=2.7 Hz), 4.13 (t, 2H, J=6.3 Hz), 2.89 (s, 3H), 1.86 (br p, 2H, J=7.2 Hz), 1.55-1.35 (m, 4H), 0.95 (br t, 3H, J=7.2 Hz).

### EXAMPLE 38

N-[4-methyl-6-(4-methylphenoxy)-2-quinazolinyl]guanidine was made in the same manner as N-(6,7-dibutoxy-4-methyl-2-quinazolinyl)guanidine (see Example 1) except that 4-(4-methylphenoxy)aniline was used in place of 3,4-dibutoxyaniline.

Name: 2,2,4-trimethyl-6-(4-methylphenoxy)-1,2-dihydroquinoline (synthesized using Method B (27% yield)).

Data: ESMS 280 (MH⁺).

Compound 1033A (class: Quinazolino-guanidine; synthesized using Method C (9% yield)).

Name: N-[4-methyl-6-(4-methylphenoxy)-2-quinazolinyl]guanidine.

Data: ESMS 308 (MH⁺); ¹H NMR (CD₃OD) δ 7.89 (d, 1H, J=9.0 Hz), 7.86 (s, 1H), 7.62 (dd, 1H, J=9.0, 2.7 Hz), 7.47 (d, 1H, J=2.4 Hz), 7.23 (d, 2H, J=8.1 Hz), 6.97 (d, 2H, J=8.4 Hz), 2.75 (s, 3H), 2.34 (s, 3H).

### EXAMPLE 39

N-(6-tert-butyl-4-methyl-2-quinazolinyl)guanidine was made in the same manner as N-(6,7-dibutoxy-4-methyl-2-quinazolinyl)guanidine (see Example 1) except that 6-tert-butylaniline was used in place of 3,4-dibutoxyaniline.

Name: 6-(tert-butyl)-2,2,4-trimethyl-1,2-dihydroquinoline.

(synthesized using method B (72% yield).

Data: ESMS 230 (MH⁺); ¹H NMR (CDCl₃) δ 6.99 (d, J=7.8 Hz, 1H), 6.66 (dd, J=7.8, 1.5 Hz, 1H), 6.46 (d, J=1.5 Hz, 1H), 5.25 (s, 1H), 3.68 (bs, 1H), 1.97(d, J=1.2 Hz, 3H), 1.28 (d, J=6.0 Hz, 6H), 1.27 (s, 6H).

Compound 1004A (class: Quinazolino-guanidine; synthesized using Method C (45% yield).

Name: N-(6-tert-butyl-4-methyl-2-quinazolinyl)guanidine.

Data: ESMS 258 (MH⁺); ¹H NMR (CD₃OD) δ 8.00-8.36 (m, 2H), 7.82 (d, J=8.7 Hz, 1H), 2.90 (s, 3H), 1.42 (s, 9H); Anal. (C₁₄H₁₉N₅. 1.1 CHCl₃. 2.4 NH₃) calcd, C, 42.22; H, 6.40; N, 24.13; Found, C, 42.13; H, 6.36; N, 24.23.

### EXAMPLE 40

N-(7-ethoxy-4-methyl-2-quinazolinyl)guanidine was made in the same manner as N-(6,7-dibutoxy-4-methyl-2-quinazolinyl)guanidine (see Example 1) except that 3-ethoxyaniline was used in place of 3,4-dibutoxyaniline.

Name: 7-ethoxy-2,2,4-trimethyl-1,2-dihydroquinoline.

(synthesized using Method B (37% yield).

Data: ¹H NMR (CDCl₃) δ 6.97 (d, J=8.4 Hz, 1H), 6.20 (dd, J=8.4, 2.4 Hz, 1H0, 6.02 (d, J=2.4 Hz, 1H), 5.19 (d, J=1.3 Hz, 1H), 3.98 (q, J=7.0 Hz, 2H), 3.53 (bs, 1H), 1.97 (d, J=1.4 Hz, 3H), 1.39 (t, J=7.0 Hz, 3H), 1.27 (s, 6H).

Compound 1024A (class: Quinazolino-guanidine; synthesized using Method C (42% yield)).

Name: N-(7-ethoxy-4-methyl-2-quinazolinyl)guanidine.

Data: ESMS 244 (MH⁺); ¹H NMR (CD₃OD) δ 8.06 (d, J=9.1 Hz, 1H), 7.44 (d, J=2.4 Hz, 1H), 7.31 (dd, J=9.1, 2.5 Hz, 1H), 4.21 (q, J=7.0 Hz, 2H), 2.83 (s, 3H), 1.46 (t, J=7.0 Hz, 3H); Anal. (C₁₂H₁₅N₅O. 1.28 CF₃CO₂H) calcd, C, 44.70; H, 4.19; N, 17.90; Found, C, 44.80; H, 4.09; N, 17.80.

### EXAMPLE 41

N-[7-(tert-butyl)-4-methyl-2-quinazolinyl]guanidine was made in the same manner as N-(6,7-dibutoxy-4-methyl-2-quinazolinyl)guanidine (see Example 1) except that 3-tert-butylaniline was used in place of 3,4-dibutoxyaniline.

Name: 7-(tert-butyl)-2,2,4-trimethyl-1,2-dihydroquinoline.

(synthesized using Method B (82% yield).

Data: ¹H NMR (CDCl₃) δ 6.99 (d, J=7.8 Hz, 1H), 6.66 (dd, J=7.8, 1.5 Hz, 1H), 6.46 (d, J=1.5 Hz, 1H), 5.25 (s, 1H), 3.68 (bs, 1H), 1.97(d, J=1.2 Hz, 3H), 1.28 (d, J=6.0 Hz, 6H), 1.27 (s, 6H).

Compound 1022A (class: Quinzolino-guanidine; synthesized using Method C (44% yield)).

Name: N-[7-(tert-butyl)-4-methyl-2-quinazolinyl]guanidine.

Data: ESMS 258 (MH⁺); ¹H NMR (CD₃OD) δ 8.09 (d, J=8.7 Hz, 1H) 7.84 (d, J=1.8 Hz, 1H), 7.72 (dd, J=8.7, 1.8 Hz, 1H), 2.86 (s, 3H), 1.41 (s, 9H); mp 195-198° C. (dec.); Anal. (C₁₄H₁₉N₅. 0.9 CH₂Cl₂. 1.2H₂O. 0.9 NH₃) calcd, C, 48.27; H, 7.04; N, 22.29; Found, C, 47.99; H, 7.04; N, 22.26.

### EXAMPLE 42

N-(6-hydroxy-4,7-dimethyl-2-quinazolinyl)guanidine was made in the same manner as N-(6,7-dibutoxy-4-methyl-2-quinazolinyl)guanidine (see Example 1) except that 6-nitro-3,4-dihydro-1(2H)-naphthalenone was used in place of 1,2-dibutoxy-4-nitrobenzene.

Name: 6-amino-1,2,3,4-tetrahydro-1-naphthalenol.

(synthesized from 6-nitro-3,4-dihydro-1(2H)-naphthalenone using Method A (67% yield).

Data: ESMS 164 (MH⁺); ¹H NMR (CDCl₃) δ 6.90 (d, 1H, J=8.1 Hz), 6.79 (d, 1H, J=2.4 Hz), 6.58 (dd, 1H, J=8.1, 2.4 Hz), 4.68 (t, 1H, J=5.4 Hz), 2.68-2.60 (m, 2H), 2.00-1.71 (m, 4H).

Compound 1017A (class: Quinazolino-guanidine; synthesized using methods B & C (28% yield over 2 steps)).

Name: N-(6-hydroxy-4,7-dimethyl-2-quinazolinyl)guanidine.

Data (CF₃CO₂H salt): ESMS 232 (MH⁺); ¹H NMR (CD₃OD) δ 7.63 (s, 1H), 7.28 (s, 1H), 2.80 (s, 3H), 2.4 (s, 3H); mp 246-248° C. (dec.); Anal. (C₁₁H₁₃N₅O. 1.25 CF₃CO₂H. 1H₂O) calcd, C, 41.39; H, 4.18; N, 17.87; Found, C, 41.52; H, 4.14; N, 17.95.

### EXAMPLE 43

N-(6-methoxy-4,7-dimethyl-2-quinazolinyl)guanidine was made in the same manner as N-(6,7-dibutoxy-4-methyl-2-quinazolinyl)guanidine (see Example 1) except that 4-methoxyaniline was used in place of 3,4-dibutoxyaniline.

Name: 6-methoxy-2,2,4,7-tetramethyl-1,2-dihydroquinoline.

(Synthesized using Method B (82% yield)).

Data: ESMS 218 (MH⁺).

Compound 1016A (class: Quinazolino-guanidine; synthesized using Method C (41% yield)).

Name: N-(6-methoxy-4,7-dimethyl-2-quinazolinyl)guanidine.

Data: ESMS 244 (MH⁺); ¹H NMR (CD₃OD) δ 7.63 (s, 1H), 7.30 (s, 1H), 3.98 (s, 3H), 2.86 (s, 3H), 2.39 (s, 3H)

### EXAMPLE 44

N-(4-methyl-8,9-dihydrobenzo[g]quinazolin-2-yl)guanidine was made in the same manner as N-(6,7-dibutoxy-4-methyl-2-quinazolinyl)guanidine (see Example 1) except that 7-nitro-1-tetralone was used in place of 1,2-dibutoxy-4-nitrobenzene.

Compound 1037A (class: Quinazolino-guanidine; synthesized using Method C (11% yield)).

Name: N-(4-methyl-8,9-dihydrobenzo[g]quinazolin-2-yl)guanidine.

Data: ESMS 254 (MH⁺); ¹H NMR (CD₃OD) δ 7.89 (s, 2H), 7.77 (s, 1H), 7.36 (s, 1H), 6.66 (d, 1H, J=9.6 Hz), 6.36 (dt, 1H, J=9.3, 4.5 Hz), 2.97 (br t, 2H), J=7.5 Hz), 2.80 (br s, 3H), 2.45-2.37 (m, 2H).

### EXAMPLE 45

N-(4-methyl-7,8-dihydro-6H-cyclopenta [g] quinazolin-2-yl)guanidine was made in the same manner as N-(6,7-dibutoxy-4-methyl-2-quinazolinyl)guanidine (see Example 1) except that 5-aminoindane was used in place of 3,4-dibutoxyaniline.

Name: 2,2, 4-trimethyl-2, 6,7, 8-tetrahydro-1H-cyclopenta[g]quinoline (synthesized using Method B (93% yield)

Data: ESMS 214 (MH⁺); ¹H NMR (CDCl₃) δ 6.96 (s, 1H), 6.38 (s, 1H), 5.28 (d, 1H, J=0.6 Hz), 2.80 (t, 4H, J=7.2 Hz), 2.16 (br t, 1H, J=7.5 Hz), 2.03 (br t, 1H), 1.99 (br d, 3H, J=0.9 Hz), 1.27 (s, 6H).

Compound 1038A (class: Quinazolino-guanidine; synthesized using Method C (18% yield)).

Name: N-(4-methyl-7,8-dihydro-6H-cyclopenta[g]quinazolin-2-yl)guanidine.

Data: ESMS 242 (MH⁺); ¹H NMR (CD₃OD) δ 7.96 (s, 1H), 7.66 (s, 1H), 3.09 (dd, 4H, J=6.9, 6.0 Hz), 2.86 (s, 3H) 2.20 (p, 2H, J=7.5 Hz); mp 295-298° C. (dec.).

### EXAMPLE 46

N-4-methyl-6-[(5-phenoxypentyl)oxy]-2-quinazolinylguanidine was made in the same manner as N-(6,7-dibutoxy-4-methyl-2-quinazolinyl)guanidine (see Example 1) except that 4-[(5-phenoxypentyl)oxy]aniline was used in place of 3,4-dibutoxyaniline.

Name: 2,2,4-trimethyl-6-[(5-phenoxypentyl)oxy]-1,2-dihydroquinoline (synthesized using Method B).

Data: 352 (ESMS, MH⁺).

Compound 1005A (class: Quinazolino-guanidine; synthesized using Method C (12% yield)).

Name: N-4-methyl-6-[(5-phenoxypentyl)oxy]-2-quinazolinylguanidine.

Data: ESMS 379 (MH⁺); ¹H NMR (CD₃OD) δ 7.79 (d, J=9.2 Hz, 1H,), 7.54 (dd, J=9.2, 2.6 Hz, 1H), 7.38 (d, J=2.5 Hz, 1H), 7.21 (t, J=8.0 Hz, 2H), 6.82-6.90 (m, 3H), 4.15 (t, J=6.2 Hz, 2H), 3.98 (t, J=6.2 Hz, 2H), 2.86 (3H, s), 1.62-2.00 (m, 6H)

### EXAMPLE 47

N-(6-butyl-4-methyl-2-quinazolinyl)guanidine was made in the same manner as N-(6,7-dibutoxy-4-methyl-2-quinazolinyl)guanidine (see Example 1) except that 4-butylaniline was used in place of 3,4-dibutoxyaniline.

Name: 6-butyl-2,2,4-trimethyl-1,2-dihydroquinoline.

(synthesized using Method B (14% yield)).

Data: ESMS 230 (MH⁺); ¹H NMR (CDCl₃) δ 6.93 (s, 1H), 6.86 (d, 1H, J=8.1 Hz), 6.42 (d, 1H, J=7.8 Hz), 5.35 (br s, 1H), 2.54 (t, 2H, J=7.5 Hz), 2.04 (s, 3H), 1.60 (p, 2H, J=7.5 Hz), 1.40 (septet, 2H, J=7.2 Hz), 1.304 (s, 3H), 1.301 (s, 3H), 0.97 (t, 3H, J=7.2 Hz).

Compound 2004A (class: Quinazolino-guanidine; synthesized using Method C (44% yield)).

Name: N-(6-butyl-4-methyl-2-quinazolinyl)guanidine.

Data: ESMS 258 (MH⁺); ¹H NMR (CD₃OD) δ 7.92 (s, 1H, 2 nd order coupling), 7.77 (s, 2H, 2^{nd} order coupling), 2.88 (s, 3H), 2.80 (t, 2H, J=7.5 Hz), 1.67 (p, 2H, J=7.8 Hz), 1.39 (septet, 2H, J=7.5 Hz), 0.95 (t, 3H, J=7.2 Hz).

### EXAMPLE 48

N-(6-benzyl-4-methyl-2-quinazolinyl)guanidine was made in the same manner as N-(6,7-dibutoxy-4-methyl-2-quinazolinyl)guanidine (see Example 1) except that 4-benzylaniline was used in place of 3,4-dibutoxyaniline.

Name: 6-benzyl-2,2,4-trimethyl-1,2-dihydroquinoline.

(synthesized using Method B (41% yield)).

Data: ESMS 263 (MH⁺); ¹H NMR (CDCl₃) δ 7.14 (t, 2H, J=7.5 Hz), 7.35-7.33 (m, 3H), 7.07 (s, 1H), 6.95 (d, 1H, J=7.8 Hz), 6.51 (dd, 1H, J=8.1, 0.9 Hz), 5.45 (br s, 1H), 4.02 (s, 2H), 2.11 (s, 3H), 1.399 (s, 3H), 1.395 (s, 3H).

Compound 2003A (class: Quinazolino-guanidine; synthesized using Method C (19% yield)).

Name: N-(6-benzyl-4-methyl-2-quinazolinyl)guanidine.

Data: ESMS 298 (MH⁺); ¹H NMR (DMSO-d₆) δ 7.62 (br s, 1H), 7.44 (d, 1H, J=8.4 Hz), 7.33 (d, 1H, J=8.1 Hz), 7.22-7.06 (m, 5H), 3.93 (s, 2H), 2.56 (s, 3H).

### EXAMPLE 49

N-(6-hexyl-4-methyl-2-quinazolinyl)guanidine was made in the same manner as N-(6,7-dibutoxy-4-methyl-2-quinazolinyl)guanidine (see Example 1) except that 4-hexylaniline was used in place of 3,4-dibutoxyaniline.

Name: 6-hexyl-2,2,4-trimethyl-1,2-dihydroquinoline.

(synthesized using Method B (329 yield)).

Data: ESMS 258 (MH⁺); ¹H NMR (CDCl₃) δ 7.12 (s, 1H), 7.08 (d, 7.8 Hz), 6.55 (dd, 1H, J=7.8, 1.2 Hz), 5.50 (d, 1H, J=1.2 Hz), 2.73 (t, 2H, J=7.2 Hz), 2.21 (d, 3H, J=1.2 Hz), 1.82 (br t, 2H, J=6.0 Hz), 1.55 (br s, 6H) 1.45 (s, 3H), 1.44 (s, 3H), 1.14 (br s, 3H).

Compound 2005A (class: Quinazolino-guanidine; synthesized using Method C (5% yield)).

Name: N-(6-hexyl-4-methyl-2-quinazolinyl)guanidine.

Data: ESMS 286 (MH); ¹H NMR (CD₃OD) δ 7.88 (s, 1H), 7.86 (s, 1H, 2^{nd} order coupling), 7.73 (br s, 2H, 2^{nd} order coupling), 2.84 (s, 3H), 2.77 (t, 2H, J=7.8 Hz), 1.6 (br s, 2H), 1.40-1.25 (m, 6H), 0.87 (br t, 3H, J=6.9 Hz).

### EXAMPLE 50

N-[7-(benzyloxy)-4-methyl-2-quinazolinyl]guanidine was made in the same manner as N-(6,7-dibutoxy-4-methyl-2-quinazolinyl)guanidine (see Example 1) except that 3-(benzyloxy)aniline was used in place of 3,4-dibutoxyaniline.

Name: 7-(benzyloxy)-2,2,4-trimethyl-1,2-dihydroquinoline.

(synthesized using Method B (72% yield)).

Data: ¹H NMR (CDCl) δ 7.34-7.52 (m, 5H), 7.04 (d, J=8.4 Hz, 1H), 6.34 (dd, J=8.4, 2.4 Hz, 1H), 6.16 (d, J=2.4 Hz, 1H), 5.26 (d, J=0.9 Hz, 1H), 5.06 (s, 2H), 3.62 (bs, 1H), 2.02 (d, J=0.9 Hz, 3H), 1.32 (s, 6H).

Compound 1006A (class: Quinazolino-guanidine; synthesized using method C (43% yield)).

Name: N-[7-(benzyloxy)-4-methyl-2-quinazolinyl]guanidine.

Data: ESMS 308 (MS+); ¹H NMR (CD₃OD) δ 8.01 (d, J=9.0 Hz, 1H), 7.17-7.48 (m, 6H), 7.20 (dd, J=9.0, 2.4 Hz, 1H), 5.20 (s, 2H), 2.78 (s, 3H); mp 215-217° C. (dec.); Anal. (C₁₇H₁₇N₅O.CF₃CO₂H. 0.2 CHCl,) calcd, C, 52.61; H, 4.23; N, 15.98; Found, C, 52.63; H, 4.26; N, 16.02.

### EXAMPLE 51

N-(6-heptyl-4-methyl-2-quinazolinyl)guanidine was made in the same manner as N-(6,7-dibutoxy-4-methyl-2-quinazolinyl)guanidine (see Example 1) except that 4-heptylaniline was used in place of 3,4-dibutoxyaniline.

Name: 6-heptyl-2,2,4-trimethyl-1,2-dihydroquinoline.

(synthesized using Method B (50% yield)).

Data: ESMS 272 (MH⁺); ¹H NMR (CDCl₃) δ 6.89 (dd, 1H, J=1.5 Hz), 6.82 (dd, 1H, J=8.1, 2.1 Hz), 5.32 (br s, 1H), 2.49 (br t, 2H, J=7.5 Hz), 2.01 (d, 3H, J=1.2 Hz), 1.60-1.53 (m, 2H), 1.32-1.30 (m, 8H), 1.27 (s, 6H), 0.90 (t, 3H, J=6.9 Hz).

Compound 2006A (class: Quinazolino-guanidine; synthesized using Method C (18% yield)).

Name: N-(6-heptyl-4-methyl-2-quinazolinyl)guanidine.

Data: ESMS 300 (MH); ¹H NMP (DMSO-d₆) δ 7.87 (s, 1H), 7.67 (br s, 2H, 2^{nd} order coupling), 2.79 (s, 3H), 2.72 (t, 2H), 1.63 (br s, 2H), 1.30 (br s, 4H), 1.24 (br s, 4H), 0.84 (br t, 3H, J=6.3 Hz).

### EXAMPLE 52

N-(4-methyl-6-pentyl-2-quinolinyl)guanidine was made in the same manner as N-(6-ethyl-4-methyl-2-quinolinyl)guanidine (see Example 3) except that 4-pentylaniline was used in place of 4-ethylaniline.

Name: 3-oxo-N-(4-pentylphenyl)butanamide.

(synthesized from 4-pentylaniline using Method G (28-36% yield).

Data: ESMS 246 (MH⁺); ¹H NMR (CDCl₃) δ 9.05 (br s, 1H) 7.43 (d, 2H, J=8.4 Hz), 7.13 (d, 2H, J=8.4 Hz), 3.58 (s, 2H), 2.56 (t, 2H, J=75 Hz), 2.32 (s, 3H), 1.58 (p, 2H, J=7.2 Hz), 1.35-1.26(m, 4H), 0.88 (t, 3H, J=6.9 Hz).

Name: 4-methyl-6-pentyl-2(1H)-quinolinone.

(synthesized using Method H (76-96% yield)).

Data: ESMS 230 (MH⁺); ¹H NMR (CDCl₃) δ 11.92 (br s, 1H), 7.45 (s, 1H, 2^{nd} order coupling), 7.33 (br s, 2H, 2^{nd} order coupling), 6.57 (s, 1H), 2.68 (t, 2H, J=7.8 Hz), 2.51 (s, 3H), 1.64 (br s, 2H), 1.36 (br s, 4H), 0.90 (br s, 3H).

Name: 2-chloro-4-methyl-6-pentylquinoline.

(synthesized using Method I (33% yield)).

Data: ESMS 250 & 248 (MH⁺); ¹H NMR (CD₃OD) δ 7.83 (br s, 1H), 7.81 (d, 1H, J=8.7 Hz), 7.63 (dd, 1H, J=8.7, 2.1 Hz), 7.33 (d, 1H, J=0.9 Hz), 2.81 (t, 2H, J=7.8 Hz), 2.69 (d, 3H, J=0.9 Hz), 1.71 (br p, 2H, J=7.8 Hz), 1.38-1.33 (m, 4H), 0.90 (br t, 3H, J=6.9 Hz).

Compound 5002A (class: Quinolino-guanidine; synthesized using Method J (2% yield)).

Name: N-(4-methyl-6-pentyl-2-quinolinyl)guanidine.

Data: ESMS 271 (MH⁺); ¹H NMR (CD₃OD) δ 7.80 (d, 1H, J=8.4 Hz), 7.75 (d, 1H, J=1.2 Hz), 7.56 (dd, 1H, J=8.4, 1.8 Hz), 6.98 (br s, 1H), 2.78 (dd, 2H, J=7.8, 6.6 Hz), 2.66 (d, 3H, J=0.6 Hz), 1.69 (br p, 2H, J=7.8 Hz), 1.37-1.32 (m, 4H), 0.89 (br t, 3H, J=6.6 Hz).

### EXAMPLE 53

N-(4-methyl-6-propyl-2-quinazolinyl)guanidine was made in the same manner as N-(6,7-dibutoxy-4-methyl-2-quinazolinyl)guanidine (see Example 1) except that 4-propylaniline was used in place of 3,4-dibutoxyaniline.

Name: 2,2,4-trimethyl-6-propyl-1,2-dihydroquinoline.

(synthesized using Method B (89% yield)).

Data: ESMS 216 (MH⁺); ¹H NMR (CDCl₃) δ 6.91 (d, 1H, J=1.8 Hz), 6.84 (dd, 1H, J=7.8, 1.8 Hz), 6.41 (d, 1H, J=7.8 Hz), 5.34 (d, 1H, J=1.2 Hz), 2.50 (t, 2H, J=7.5 Hz), 2.02 (d, 3H, J=1.2 Hz), 1.62 (septet, 2H, J=7.8 Hz), 1.29 (s, 6H), 0.96 (t, 3H, J=7.5 Hz).

Compound 1008A (synthesized using Method C (24% yield)).

Name: N-(4-methyl-6-propyl-2-quinazolinyl)guanidine.

Data: ESMS 244 (MH⁺); ¹H NMR (CDCl,) δ 7.64 (s, 1H, 2^{nd} order coupling), 7.58 (s, 2H, 2^{nd} order coupling), 2.80 (s, 3H), 2.68 (t, 2H, J=7.2 Hz), 1.65 (septet, 2H, J=7.5 Hz), 0.93 (t, 3H, J=8.4 Hz).

### EXAMPLE 54

N-(4-methyl-6-phenyl-2-quinazolinyl)guanidine was made in the same manner as N-(6,7-dibutoxy-4-methyl-2-quinazolinyl)guanidine (see Example 1) except that 4-phenylaniline was used in place of 3,4-dibutoxyaniline.

Name: 2,2,4-trimethyl-6-phenyl-1,2-dihydroquinoline.

(synthesized using Method B (61% yield)).

Data: ESMS 250 (MH⁺); ¹H NMR (CDCl₃) δ 7.77-7.72 (m, 2H), 7.60-7.50 (m, 3H), 7.47-7.40 (m, 2H), 6.65-6.50 (m, 1H), 5.51 (br s, 1H), 2.23 (br s, 3H), 1.44 (br s, 6H).

Compound 1010A (class: Quinazolino-guanidine; synthesized using Method C (3% yield)).

Name: N-(4-methyl-6-phenyl-2-quinazolinyl)guanidine.

Data: ESMS 278 (MH⁺); ¹H NMR (CD₃OD) δ 88.31 (d, 1H, J=1.8 Hz), 8.19 (dd, 1H, 8.7, 1.8 Hz), 7.94 (d, 1H, J=8.7 Hz), 7.75 (d, 2H, J=7.2 Hz), 7.50 (t, 2H, J=6.9 Hz), 7.40 (t, 1H, J=7.2 Hz), 2.97 (s, 3H).

### EXAMPLE 55

N-(4-methyl-6-octyl-2-quinazolinyl)guanidine was made in the same manner as N-(6,7-dibutoxy-4-methyl-2-quinazolinyl)guanidine (see Example 1) except that 4-octylaniline was used in place of 3,4-dibutoxyaniline.

Name: 2,2,4-trimethyl-6-octyl-1,2-dihydroquinoline.

(synthesized using Method B (72% yield)).

Data: ESMS 286 (MH⁺); ¹H NMR (CDCl₃) δ 6.90-6.75(m, 2H), 6.41-6.33 (m, 1H), 5.29 (br s, 1H), 2.50-2.42 (m, 2H), 2.01-1.96 (m, 3H), 1.55 (br s, 2H), 1.29-1.21 (m, 16H), 0.91-0.54 (m, 3H).

Compound 1009A (class: Quinazolino-guanidine; synthesized using Method C (12% yield)).

Name: N-(4-methyl-6-octyl-2-quinazolinyl)guanidine.

Data: ESMS 314 (MH⁺); ¹H NMR (DMSO-d₆) δ 7.79 (s, 1H, 2^{nd} order coupling), 7.62-7.50 (m, 2H, 2^{nd} order coupling), 2.732 (br s, 5H), 1.60 (br s, 2H), 1.21 (br s, 10H), 0.82 (br t, 3H).

### EXAMPLE 56

N-(6-hexyl-4-methyl-2-quinolinyl)guanidine was made in the same manner as N-(6-ethyl-4-methyl-2-quinolinyl)guanidine (see Example 3) except that 4-hexylaniline was used in place of 4-ethylaniline.

Name: N-(4-hexylphenyl)-3-oxobutanamide.

(synthesized from 4-hexylaniline using Method G (54% yield)).

Name: 6-hexyl-4-methyl-2(1H)-quinolinone.

(synthesized using Method H (100% yield)).

Data: ESMS 244 (MH⁺).

Name: 2-chloro-6-hexyl-4-methylquinoline.

(synthesized using Method I (60% yield)).

Data: ESMS 264 & 262 (MH⁺); ¹H NMR (CDCl₃) δ 7.78 (br d, 1H, J=2.4 Hz), 7.75 (s, 1H), 7.59 (dd, 1H, J=8.7, 1.5 Hz), 7.27 (br s, 1H), 2.77 (t, 2H, J=7.5 Hz), 2.64 (s, 3H), 1.67 (br p, 2H, J=7.2 Hz), 1.31 (br s, 6H), 0.86 (br t, 3H, J=6.9 Hz).

Compound 5003A (class: Quinolino-guanidine; synthesized using Method J (10% yield)).

Name: N-(6-hexyl-4-methyl-2-quinolinyl)guanidine.

Data: ESMS 285 (MH⁺); ¹H NMR (CD₃OD) δ 7.72 (d, 1H, J=8.7 Hz), 7.67 (d, 1H, J=0.9 Hz), 7.51 (dd, 1H, J=8.4, 1.8 Hz), 6.92 (br s, 1H), 2.75 (t, 2H, J=7.5 Hz), 2.60 (s, 3H), 1.67 (br p, 2H, J=7.8 Hz), 1.32 (br s, 6H), 0.88 (br t, 3H, J=6.9 Hz).

### EXAMPLE 57

N-(6-[1-(4-hydroxyl-pentyl)]-4-methyl-2-quinazolino)guanidine was made in the same manner as N-(6-ethyl-4-methyl-2-quinazolino)guanidine (see Example 1) except that 5-(4-aminophenyl)-2-pentanol was used in place of 4-ethylaniline.

Compound 1034A

Name: N-(6-[1-(4-hydroxyl-pentyl)]-4-methyl-2-quinazolino)guanidine.

Data: ESMS 288 (MH⁺); ¹H NMR (CD₃OD) δ 7.96 (s, 1H), 7.80 (s, 2H), 3.74 (p, J=6.3 Hz, 1H), 2.90 (s, 3H), 2.85-2.81 (m, 2H), 1.85-1.65 (m, 2H), 1.55-1.45 (m, 2H), 1.14 (d, J=6.3 Hz, 3H).

### EXAMPLE 58

N-(6-butyl-4-methyl-2-quinolinyl)guanidine was made in the same manner as N-(6-ethyl-4-methyl-2-quinolinyl)guanidine (see Example 3) except that 4-butylaniline was used in place of 4-ethylaniline.

Compound 5001A

Name: N-(6-butyl-4-methyl-2-quinolinyl)guanidine.

Data: ESMS 257 (MH⁺); ¹H NMR (CD₃OD) δ 7.82 (d, J=8.4 Hz, 1H), 7.78 (d, J=1.5 Hz, 1H), 7.58 (dd, J=8.4, 1.5 Hz, 1H), 6.93 (s, 1H), 2.81 (t, J=7.2 Hz, 2H), 2.68 (s, 3H), 1.69 (p, J=7.2 Hz, 2H), 1.39 (sextet, J=7.2 Hz, 2H), 0.95 (t, J=7.2 Hz, 3H).

### EXAMPLE 59

N-(4-methyl-7-phenyl-2-quinazolinyl)guanidine was made in the same manner as N-(6,7-dibutoxy-4-methyl-2-quinazolinyl)guanidine (see Example 1) except that 3-phenylaniline was used in place of 3,4-dibutoxyaniline.

Compound 1023A

Name: N-(4-methyl-7-phenyl-2-quinazolinyl)guanidine.

Data: ESMS 278 (MH⁺); ¹H NMR (CD₃OD) δ 8.17 (br s, 1H), 8.05 (br s, 1H), 7.84 (br s, 1H), 7.70 (br s, 2H), 7.43 (br s, 2H), 7.35 (br s, 1H), 2.87 (s, 3H).

### EXAMPLE 60

N-[4-methyl-7-(isopropoxy)-2-quinazolinyl]guanidine was made in the same manner as N-(6,7-dibutoxy-4-methyl-2-quinazolinyl)guanidine (see Example 1) except that 3-isopropoxyaniline was used in place of 3,4-dibutoxyaniline.

Compound 1025A

Name: N-[4-methyl-7-(isopropoxy)-2-quinazolinyl]guanidine.

Data: ESMS 260 (MH⁴); ¹H NMR (CD₃OD) δ 8.03 (d, J=9.3 Hz, 1H), 7.23 (d, J=2.4 Hz, 1H), 7.13 (dd, J 9.3, 2.4 Hz, 1H), 3.29 (septet, J=6.0 Hz, 1H), 2.81 (s, 3H), 1.39 (d, J=6.0 Hz, 6H).

Table 1. Summary of compounds prepared in Part A.

TABLE 1
Summary of compounds prepared in Part A.

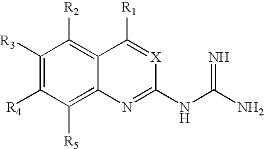

| Com- | | | | | | |
|---|---|---|---|---|---|---|
| pound | X | R₁ | R₂ | R₃ | R₄ | R₅ |
| 1001A | N | methyl | H | H | H | H |
| 1002A | N | methyl | H | methyl | H | H |
| 1003A | N | methyl | H | ethyl | H | H |
| 1004A | N | methyl | H | tert-butyl | H | H |
| 1005A | N | methyl | H | 5-phenoxy- | H | H |
| | | | | pentoxy | | |
| 1006A | N | methyl | H | H | benzyloxy | H |
| 1007A | N | methyl | fused benzene | | H | H |
| 1008A | N | methyl | H | propyl | H | H |
| 1009A | N | methyl | H | octyl | H | H |
| 1010A | N | methyl | H | phenyl | H | H |
| 1011A | N | methyl | H | OMe | H | H |
| 1012A | N | methyl | H | OBu | H | H |
| 1013A | N | methyl | H | Cl | H | H |
| 1014A | N | methyl | H | H | Br | H |
| 1015A | N | methyl | H | methyl | methyl | H |
| 1016A | N | methyl | H | OMe | methyl | H |
| 1017A | N | methyl | H | OH | methyl | H |
| 1018A | N | methyl | H | OBu | OBu | H |
| 1019A | N | methyl | H | F | F | H |
| 1020A | N | methyl | H | H | ethyl | H |
| 1021A | N | methyl | H | H | iso-propyl | H |
| 1022A | N | methyl | H | H | tert-butyl | H |
| 1023A | N | methyl | H | H | phenyl | H |
| 1024A | N | methyl | H | H | OEt | H |
| 1025A | N | methyl | H | H | isopropyl | H |
| 1026A | N | methyl | H | Br | H | H |
| 1027A | N | ethyl | H | methyl | H | H |
| 1028A | N | methyl | H | benzyloxy | H | H |
| 1029A | N | methyl | H | cyclohexyl | H | H |
| 1030A | N | methyl | H | OCF₃ | H | H |
| 1031A | N | methyl | H | penzyloxy | H | H |
| 1032A | N | methyl | H | OPh | H | H |
| 1033A | N | methyl | H | 4-methyl- | H | H |
| | | | | phenyloxy | | |
| 1034A | N | methyl | H | 4-hydroxy- | H | H |
| | | | | pentyl | | |
| 1035A | N | methyl | H | H | 1-hydroxy- | H |
| | | | | | ethyl | |
| 1036A | N | methyl | H | H | OCF₃ | H |
| 1037A | N | methyl | H | fused 5,6-cyclohexenyl | | H |
| 1038A | N | methyl | H | fused cyclopentyl | | H |
| 1039A | N | methyl | H | fused 2,3-furyl | | H |
| 2001A | N | methyl | H | pentyl | H | H |
| 2002A | N | methyl | H | sec-butyl | H | H |
| 2003A | N | methyl | H | benzyl | H | H |
| 2004A | N | methyl | H | butyl | H | H |
| 2005A | N | methyl | H | hexyl | H | H |
| 2006A | N | methyl | H | heptyl | H | H |
| 3001A | N | methyl | H | H | methyl | H |
| 4001A | C | methyl | H | methyl | H | H |
| 4002A | C | methyl | H | ethyl | H | H |
| 4003A | C | methyl | H | Ph | H | H |
| 4004A | C | methyl | H | OMe | H | H |
| 4005A | C | methyl | H | Cl | H | H |
| 4006A | C | methyl | H | H | methyl | H |
| 4007A | C | methyl | H | H | F | H |
| 4008A | C | methyl | methyl | H | methyl | H |
| 4009A | C | methyl | fused benzene | | H | H |
| 5001A | C | methyl | H | butyl | H | H |
| 5002A | C | methyl | H | pentyl | H | H |
| 5003A | C | methyl | H | hexyl | H | H |
| 6001A | C | methyl | H | H | H | H |
| 6002A | C | methyl | H | H | H | methyl |
| 6003A | C | ethyl | H | H | methyl | H |

Part B. Peptide and Peptidomimetic Compounds Sulfonylamide Compounds

Compounds described in Part B are labeled with the suffix “B”.

General Methods for Part B:

All solution-phase reactions were performed under an inert atmosphere (argon) and the reagents, neat or in appropriate solvents, were transferred to the reaction vessel via syringe and cannula techniques. The solid phase synthesis reactions were performed in vials using J-KEM heating shakers (Saint Louis, Mo.). All amino acid derivatives used as starting materials were purchased from Calbiochem-Novabiochem (San Diego, Calif.). Anhydrous solvents were purchased from Aldrich Chemical Company and used as received. The compounds described were named using ACD/Name program (version 2.51, Advanced Chemistry Development Inc., Toronto, Ontario, M5H2L3, Canada). The ¹H and ¹³C spectra were recorded at 300 and 75 MHz, respectively (QE-300 Plus by GE, Fremont, Calif.). Chemical shifts are reported in parts per million (ppm) and referenced with respect to the residual proton (i.e. CHCl₃, CHD₂OD) of the deuterated solvent. Splitting patterns are designated as s=singlet; d=doublet; t=triplet; q=quartet; p=quintet; sextet; septet; dd=doublet of a doublet; b=broad; m=multiplet. Elemental analyses were performed by Robertson Microlit Laboratories, Inc. Low-resolution electrospray mass spectra (ESMS) were measured on a Platform II instrument (Fisons, Manchester, UK) and MH⁺ is reported. Thin-layer chromatography (TLC) was carried out on glass plates precoated with silica gel 60 F₂₅₄ (0.25 mm, EM Separations Tech.). Preparative TLC was carried out on glass sheets precoated with silica gel GF (2 mm, Analtech). Flash column chromatography was performed on Merck silica gel 60 (230-400 mesh). The structures of the final products were confirmed by standard analytical methods such as elemental analysis and spectroscopic characteristics such as MS, NMR, analytical HPLC.

Synthesis:

The compounds of the present invention may be synthesized by the routes shown in Schemes 4 and 5, or with appropriate modifications as described herein. In Method 1, and Method 2, the product is isolated at the end of the synthesis, and purified by a suitable procedure such as high performance liquid chromatography (HPLC), crystallization, column chromatography, thin layer chromatography, etc. While preferred reactants have been identified herein, it is further contemplated that the present invention would include chemical equivalents to each reactant(s) specifically enumerated in this disclosure.

Two general procedures were used in the synthesis of the specific sulfonamides described above. They are described by using 1-naphthalenesulfonylamido-Arg-Phe-amide as an example:

Method I: Solid Phase Synthesis:

The general scheme for the solid phase synthesis is shown in Scheme 4.

General Experimental Procedure:

Rink amide MBHA resin (1.85 g, 1 mmol, 0.54 mmol/g, Novabiochem, San Diego, Calif., #01-64-0013) was swelled in a mixture of N,N-dimethylformamide (DMF), and N-methylpyrrolidone (NMP) (1:1, 25 mL) in a glass column with a sintered glass frit, on a platform shaker, for 10 min. The solvents were drained and the resin was treated with 30% piperidine in DMF (25 mL) for 5 min. and the liquid was drained. The piperidine treatment was repeated for 25 min. The resin was then washed, for 5 min. per wash, with DMF:NMP (1:1, 25 mL, three times), followed by methanol (25 mL, two times) and DMF:NMP (1:1, 25 mL, three times). The resin was then treated with a pre-mixed solution of Fmoc-L-phenylalanine (1.54 g, 4 mmol), HBTU (1.5 g, 4 mmol) and diisopropylethylamine (1.4 mL, 8 mmol). The resin slurry was shaken for 2 h. After draining of the amino acid solution, the resin was washed three times with DMF:NMP (1:1, 25 mL). The resin was treated with 30% piperidine in DMF (25 mL) for 5 min. and the liquid was drained. The piperidine treatment was repeated for 25 min. The resin was then washed, for 5 min. per wash with DMF:NMP (1:1, 25 mL, three times), followed by methanol (25 mL, two times) and DMF:NMP (1:1, 25 mL, three times). The resin was then treated with a pre-mixed solution of Fmoc-L-arginine(Pbf) (2.6 g, 4 mmol) with HBTU (1.5 g, 4 mmol) and diisopropylethyl amine (1.4 mL, 8 mmol). The resin slurry was shaken for 2 h. After draining of the amino acid solution, the resin was washed three times with DMF:NMP (1:1, 25 mL). The resin was treated with 30% piperidine in DMF (25 mL) for 5 and 25 min, respectively, as described above. The resin was then washed, for 5 min. each, with DMF:NMP (1:1, 25 mL, three times), followed by methanol (25 mL, two times) and DMF:NMP (1:1, 25 mL, three times). To the resin was then added 1-naphthalenesulfonyl chloride (0.53 g, 2 mmol), and triethylamine (0.56 mL, 4 mmol) in DMF (10 mL). After shaking for 3 h, the reagents were drained, and the resin was washed for 5 min. per wash, with DMF:NMP (1:1, 25 mL, three times), followed by methanol (25 mL, two times) and vacuum dried. The product was cleaved from the resin with trifluoroacetic acid:dithioethane:anisole:thioanisole:m-cresol:water triisopropylsilane (78:5:3:3:3:5:3, 25 mL) for 2 h and the cleavage solution was filtered. The filtrate was evaporated to an oil, and anhydrous ether was added to precipitate the product, which was filtered, washed with ether, and vacuum dried to yield the crude product (286 mg, 45.6%). The product was purified by using reverse phase preparative HPLC (250×22.5 mm, Primesphere C18-HC) with a gradient of 10-70% acetonitrile (0.1% TFA) in water (0.1% TFA) over 30 min (25 mL/min flow rate, detection at 215 nm). The fractions containing the product were pooled and lyophilized to yield the product (107 mg).

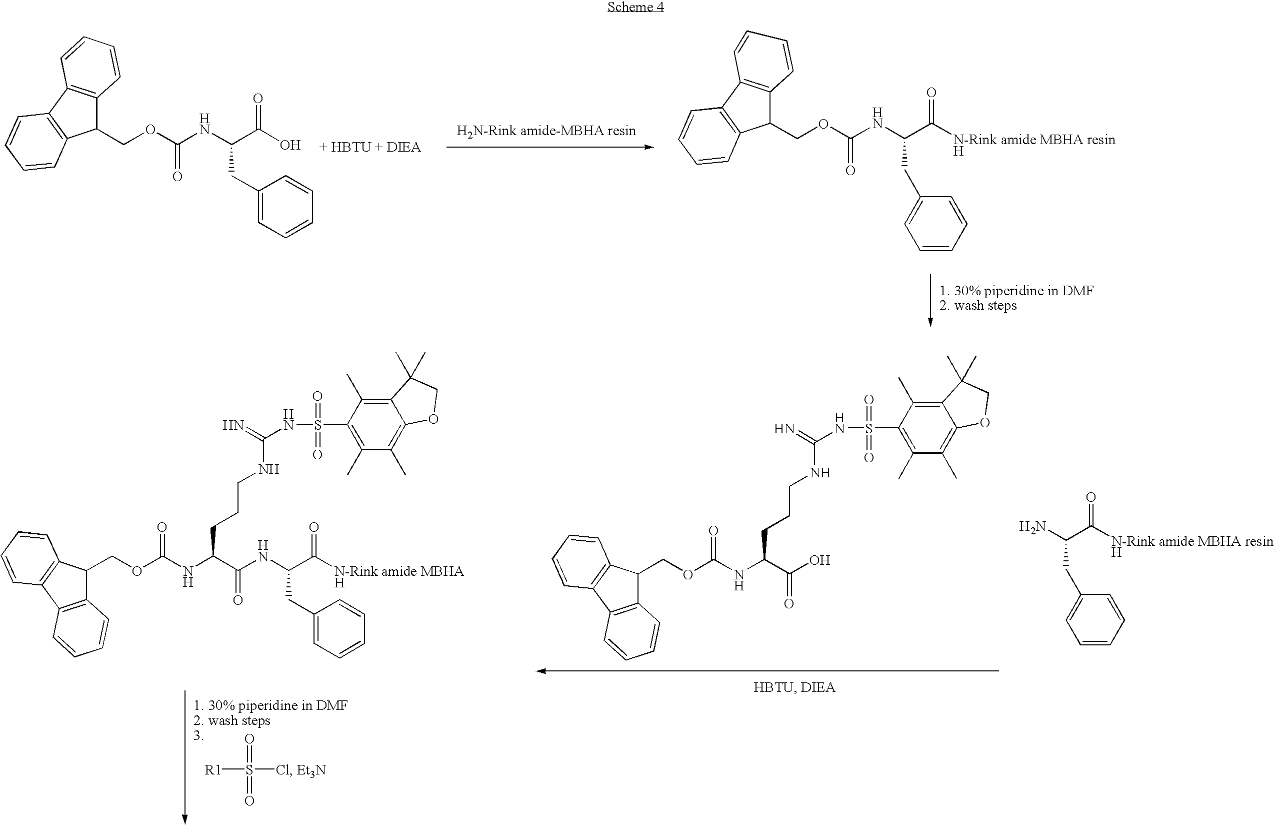

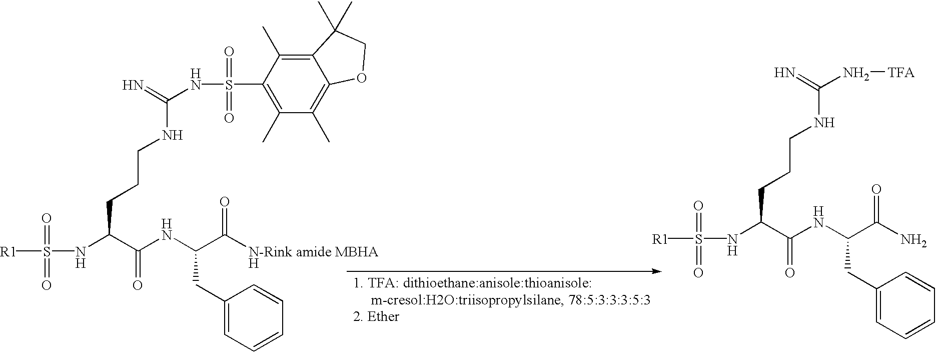

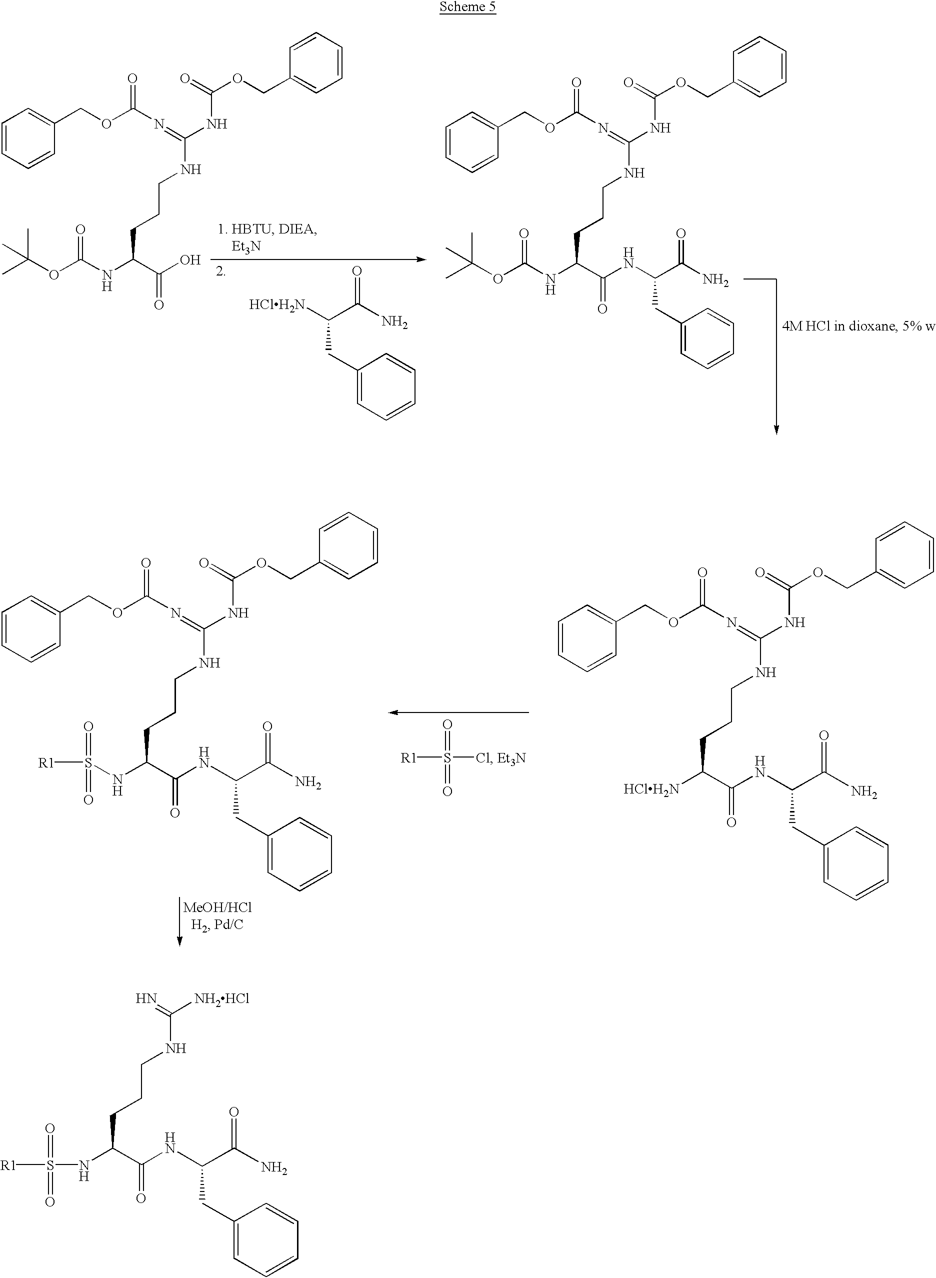

Method 2. Solution-Phase Synthesis.

Experimental Procedures for Method 2.

(N^{α}-Boc)arginine(diZ)-phenylalaninamide: (Z=benzyloxy carbonyl):

(N^{α}-Boc)-arginine(diZ)-OH (4.8 g, 8.85 mmol) was suspended in dichloromethane (100 mL), and N,N-dimethylformamide (DMF) was added dropwise while stirring, until a clear solution was obtained (10 mL) To this solution was added HBTU (3.4 g, 8.85 mmol) in DMF (20 mL). Triethylamine (1.3 mL, 8.85 mmol) was added and the solution was stirred for 5 min. To this was added a mixture of L-phenylalaninamide.HCl (1.8 g, 8.85 mmol) in dichloromethane (25 mL), containing triethylamine (3.7 mL, 26.55 mmol). The reaction mixture was stirred overnight. The volatiles were evaporated in a rotary evaporator at 45° C. The residue was dissolved in ethylacetate (200 mL) and washed with water, saturated aq. NaHCO₃, water, sat. aq. NaCl and dried (Na₂SO₄). Evaporation of the solvent gave the crude product, which was crystallized from ethyl acetate: 5.4 g (90%); m.p. 122-124° C. (dec.);

H-Arginine(diZ)-phenylalaninamide.HCl:

(N^{α}-Boc)arginine(diz)-phenylalaninamide (3.3 g), was dissolved in THF (20 mL), and treated with 4M HCl in dioxane (20 mL) for 20 min. The solvent was evaporated to dryness. The residue was treated with anhydrous ether and triturated. The precipitated product was filtered and washed with ether, and vacuum dried: 2.1 g (72%).

In the final step, 1-naphthalenesulfonyl chloride (2 eq.) was coupled with H-Arginine(diZ)-phenylalaninamide.HCl, with 4 eq. of triethylamine in THF for 4-6 h. The reaction mixtur was evaporated to dryness, and partitioned between ethyl acetate and sat. aq. NaHCO₃. The ethyl acetate layer was washed with water, sat. aq. NaCl and dried (Na₂SO₄). Filtration and evaporation of the ethyl acetate led to the protected compound. The Z groups were removed by hydrogenation with Pd/C (5%) as the catalyst, in ethanol, with 0.5% V/V conc. HCl. The product was purified by using reverse phase preparative HPLC (250×22.5 mm, Primesphere C18-HC) with a gradient of 10%-70% acetonitrile (0.1% TFA) in water (0.1% TFA) over 30 min (25 mL/min flow rate, detection at 215 nm). The fractions containing the product were pooled and lyophilized to yield the product.

The synthesis of N-amido-substituted products (where R3 and R4 in the generic structure is a substituent other than H), can be achieved by modifying procedure 1 to accommodate the incorporation of R3 or R4 via alkylation or reductive coupling. After the coupling of the first residue (e.g., Fmoc Phenylalanine in the general procedure) to the resin followed by the removal of the Fmoc protecting group as descibed above, the resin is treated with the appropriate alkyl halide (0.9 eq.), in DMF or dichloromethane, with 2-3 eq. of triethylamine for 3-4 h. Alternately, reductive coupling with the appropriate aldehyde as described in the literature (Gordon, D. W. and Steele, J., Bioorg. Med. Chem. Lett., 5(1), 1995, 47-50), can be utilized to incorporate R4. In the next step, Fmoc-Arginine(Pbf) is coupled to the secondary amine on resin, and the Fmoc protecting group removed, again as described in the general procedure. Then, the R3 group can be introduced by methods described above, followed by the coupling of the appropriate sulfonyl chloride. Cleavage with the trifluoroacetic acid cocktail and precipitation with ether gives the purified product, which can be purified by preparative HPLC as described above.

In schemes 4 and 5, the protected forms of phenylalanine and arginine can each be replaced with appropriately protected forms of other amino acids (which can be obtained from RSP Amino Acid Analogs Inc., Boston, Mass.) in order to obtain the claimed compounds. Compounds where R2 is —(CH₂)ₙN(R7)₂ wherein at least one R7 group is H can be synthesized by using the appropriate amino acids as described above, followed by protecting group cleavage and treatment of the product with the appropriate alkylating agent(s) R7-X, (where X=Cl, Br, I), with an excess of a tertiary amine base, in a polar solvent.

For compounds where R5=OH, the synthesis can be achieved by starting with the protected phenylalanine attached to Wang resin or 2-chlorotrityl chloride resin. Cleavage with the TFA cocktail after the synthesis is complete gives the product with the C-terminal acid. For the synthesis of compounds with R5=N(R8)₂, it is preferred to first obtain the fully-protected sulfonylated compound as follows: The synthesis is performed by starting with Fmoc-phenylalanine attached to 2-chlorotritylchloride resin. Upon completion of the synthesis, the protected compound is obtaining by cleaving it from the resin with 1% TFA in dichloromethane. The cleavage solution is neutralized with pyridine in methanol, and evaporated. The crude compound containing a C-terminal acid is then coupled to an appropriate amine ((R8)₂NH) by using a coupling procedure similar to that described in Method 2, to give the substituted amide.

Compound 1001B

N1-[(ls)-2-Amino-1-benzyl-2-oxoethyl]-(2S)-(5-guanidino)-2-[(1-naphthylsulfonyl)amino]pentanamide (1). (Alternate name: 1-naphthalenesulfonylamido-Arg-Phe-NH₂).

This compound was synthesized according to Method 1 described above.

Data: ESMS 511(MH⁺); ¹H NMR (CD₃OD) δ 8.65 (d, J=8.1 Hz, 1H), 8.13 (t, J=6.9 Hz, 2H), 8.01 (m, 2H), 7.64 (m, 2H) 7.52 (t, J=9.0 Hz), 7.05-7.2 (m, 4H), 4.30 (q, J=6.3, 6.0 Hz, 1H), 3.59 (m, 1H), 2.91 (dd, J=7.2, 9.6 Hz), 2.79 (m, 2H), 2.63 (m, 1H), 1.43 (m, 2H), 1.25 (m, 1H), 1.16 (m, 1H); ¹³C NMR (CD₃OD) d 24.86, 30.07, 37.85, 40.67, 54.69, 56.66, 104.75, 124.49, 124.51, 126.98, 127.28, 128.43, 128.59, 129.34, 134.98, 137.36, 158.02, 172.28, 174.77;

Anal. C₂₅H₃₀N₆O₄S+1.75 CF₃COOH calcd. C, 48.20%; H, 4.51%; N, 11.83%; S, 4.52%; found C, 48.08%; H, 4.51%; N, 11.91%; S, 4.64%; [a]_{D}=−29.8 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 18.9 min;

Compound 1002B

N1-[(S)-2-Amino-1-benzyl-2-oxoethyl]-(2S)-{[amino(imino)methyl]amino}-2-[(3-nitrophenyl)sulfonyl]amino}pentanamide.

(Alternate name: 3-Nitrophenylsulfonylamido-Arg-Phe-NH₂).

This compound was synthesized as described in Method 1, except that 3-nitrophenylsulfonyl chloride (442 mg, 2 mmol) was used in place of 1-naphthalenesulfonyl chloride.

Data: ESMS 506(MH⁺);

Compound 1003B

N1-[(1S)-2-Amino-1-benzyl-2-oxoethyl]-(2S)-{[amino(imino)methyl]amino}-2-[(4-nitrophenyl)sulfonyl]amino}pentanamide.

(Alternate name: 4-Nitrophenylsulfonylamido-Arg-Phe-NH₂).

This compound was synthesized as described in Method 1, except that 4-nitrophenylsulfonyl chloride (442 mg, 2 mmol) was used in place of 1-naphthalenesulfonyl chloride.

Data: ESMS 506 (MH⁺) Compound 1004B

N1-[(1S)-2-Amino-1-benzyl-2-oxoethyl]-(2S)-{[amino(imino)methyl]amino}-2-[(2, 6-difluorophenyl)sulfonyl]amino}pentanamide. (Alternate name: 2,6-Difluorophenylsulfonylamido-Arg-Phe-NH₂).

This compound was synthesized as described in Method 1, except that 2,6-dichlorophenylsulfonyl chloride (425.2 mg, 2 mmol) was used in place of 1-naphthalenesulfonyl chloride.

Data: ESMS 497(MH⁺);

Compound 1005B

N1-[(1S)-2-Amino-1-benzyl-2-oxoethyl]-(2S)-{[amino(imino)methyl]amino}-2-[(4-fluorophenyl)sulfonyl]amino}pentanamide.

(Alternate name: 4-Fluorophenylsulfonylamido-Arg-Phe-NH₂).

This compound was synthesized as described in Method 1, except that 4-fluorophenylsulfonyl chloride (389.2 mg, 2 mmol) was used in place of 1-naphthalenesulfonyl chloride.

Data: ESMS 479(MH⁺);

Compound 1006B

N1-[(1S)-2-Amino-1-benzyl-2-oxoethyl]-(2S)-{[amino(imino)methyl]amino}-2-[(4-chlorophenyl)sulfonyl]amino}pentanamide.

(Alternate name: 4-Chlorophenylsulfonylamido-Arg-Phe-NH₂).

This compound was synthesized as described in Method 1, except that 4-chlorophenylsulfonyl chloride (422.14 mg, 2 mmol) was used in place of 1-naphthalenesulfonyl chloride.

Data: ESMS 495(MH⁺);

Compound 2001B

N1-[(1S)-2-Amino-1-benzyl-2-oxoethyl]-(2S)-{[amino(imino)methyl]amino}-2-[(2-bromophenyl)sulfonyl]amino}pentanamide.

(Alternate name: 2-Bromophenylsulfonylamido-Arg-Phe-NH₂).

This compound was synthesized as described in Method 1, except that 2-bromophenylsulfonyl chloride (511.04 mg, 2 mmol) was used in place of 1-naphthalenesulfonyl chloride.

Data: ESMS 539(MH⁺);

Compound 1007B

N1-[(1S)-2-Amino-1-benzyl-2-oxoethyl]-(2S)-{[amino(imino)methyl]amino}-2-[(p-tolyl)sulfonyl]amino}pentanamide.

(Alternate name: p-Tolylsulfonylamido-Arg-Phe-NH₂).

This compound was synthesized as described in Method 1, except that 4-methylphenylsulfonyl chloride (381.3 mg, 2 mmol) was used in place of 1-naphthalenesulfonyl chloride.

Data: ESMS 475(MH⁺);

Compound 1008B

N1-[(1S)-2-Amino-1-benzyl-2-oxoethyl]-(2S)-{[amino(imino)methyl]amino}-2-[phenylsulfonyl]amino}pentanamide.

(Alternate name: Phenylsulfonylamido-Arg-Phe-NH₂).

This compound was synthesized as described in Method 1, except that phenylsulfonyl chloride (353.24 mg, 2 mmol) was used in place of 1-naphthalenesulfonyl chloride.

Data: ESMS 461(MH⁺); Compound 1009B

N1-[(1S)-2-Amino-1-benzyl-2-oxoethyl]-(2S)-{[amino(imino)methyl]amino}-2-[(4-methoxyphenyl) sulfonyl]amino)pentanamide.

(Alternate name: 4-Methoxyphenylsulfonylamido-Arg-Phe-NH₂).

This compound was synthesized as described in Method 1, except that 4-methoxyphenylsulfonyl chloride (413.3 mg, 2 mmol) was used in place of 1-naphthalenesulfonyl chloride.

Data: ESMS 491(MH⁺);

Compound 1010B

N1-[(1S)-2-Amino-1-benzyl-2-oxoethyl]-(2S)-{[amino(imino)methyl]amino}-2-[(2,4-dichlorophenyl)sulfonyl]amino}pentanamide. (Alternate name: 2,4-Dichlorophenylsulfonylamido-Arg-Phe-NH₂).

This compound was synthesized as described in Method 1, except that 2,4-dichlorophenylsulfonyl chloride (491.02 mg, 2 mmol) was used in place of 1-naphthalenesulfonyl chloride.

Data: ESMS 529(MH⁺); ¹H NMR (CD₃OD) d 8.13 (d, J=7.88 Hz, 1H), 7.87 (d, J=8.4 Hz, 1H), 7.61 (d, J=2.02 Hz, 1H) 7.37 (dd, J=2.7, 3.7 Hz, 2H), 7.25 (m, 4H), 4.35 (m, 1H) 3.75 (q, J 1.77, 5.75 Hz, 1H), 3.04 (m, 2H), 2.96 (m, 1H) 2.78 (m, 1H), 1.44-1.65 (m, 4H); ¹³C NMR (CD₃OD) d 25.01, 30.42, 38.09, 40.93, 54.90, 56.78, 127.05, 127.77, 128.69, 129.49, 131.84, 132.41, 133.46, 139.71, 157.79, 171.84, 174.84; [a]=+7.0 (c=1% W/V in methanol);

Anal. C₂₁H₂₆Cl₂N₆O₄S+1.5 CF₃COOH calc. C, 41.15%; H, 3.96%; N, 12.00%; Cl, 10.12%; S, 4.58%; found C, 41.46%; H, 4.00%; N, 12.37%; Cl, 9.98%; S, 4.80%.

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 1011B

N1-[(1S)-2-Amino-1-benzyl-2-oxoethyl]-(2S)-5-[amino(imino)methyl]amino-2-[(benzylsulfonyl)amino]pentanamide.

Alternate name: α-Toluenesulfonamido-Arg-Phe-NH2

This compound was synthesized as described in Method 1, except that a-toluenesulfonyl chloride (379.3 mg, 2 mmol) was used in place of 1-naphthalenesulfonyl chloride.

Data: ESMS 475 (MH⁺); ¹H NMR (CD₃OD) d 7.317-7.16 (m, 10H), 7.06 (t, J=8.0 Hz, 1H), 4.69 (q, J=5.0, 4.8 Hz, 1H), 4.11 (m, 2H), 3.75 (m, 2H), 3.17(m, 1H), 3.05 (t, J=6.9 Hz, 2H), 2.87 (m, 2H), 1.55 (m, 2H), 1.44 (m, 2H), 1.28 (t, J=7.3 Hz,1H); ¹³C NMR (CD₃OD) d 8.38, 24.96, 30.60, 38.04, 40.95, 54.75, 56.92, 58.92, 104.98, 127.06, 128.71, 128.73, 129.48, 129.87, 131.28, 137.74, 157.83, 172.83, 175.21; [a]_{D}=−5.0 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 21.7 min;

Compound 1012B

N1-[(1S)-2-Amino-1-benzyl-2-oxoethyl]-(2S)-{[amino(imino)methyl]amino}-2-[4-iodophenyl) sulfonyl]amino}pentanamide.

(Alternate name: 4-Iodophenylsulfonylamido-Arg-Phe-NH₂).

This compound was synthesized as described in Method 1, except that 4-iodophenylsulfonyl chloride (605.04 mg, 2 mmol) was used in place of 1-naphthalenesulfonyl chloride.

Data: ESMS 506(MH⁺);586.99 ¹H NMR (CD₃OD) d 1.29 (t, J=7.3 Hz, 1H), 1.44 (m, 2H), 1.55 (m, 2H), 2.73 (dd, J=8.8, 4.9 Hz, 1H), 3.02 (m, 2H), 3.20 (q, 1H), 3.71 (t, J=6 Hz, 1H), 4.3 (q, J=6.0, 2.86 Hz), 7.34 (m, 5H), 7.45 (d, J=8.6 Hz, 2H), 7.80 (d, J=8.6 Hz, 2H); [a]D=+5.7 (c=1% W/V in methanol);

Anal. C₂₁H₂₇IN₆O₄S+1.25 CF₃COOH calcd. C, 38.72%; H, 3.91%; N, 11.53%; S, 4.40%; found C, 38.51%; H, 3.75%; N, 11.07%; S, 4.49%;

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.7 min;

Compound 1013B

N1-[(S)-2-Amino-1-benzyl-2-oxoethyl]-(2S)-{[amino(imino)methyl]amino}-2-[(2-thiophene)sulfonyl]amino)pentanamide.

(Alternate name: 2-Thiophenesulfonylamido-Arg-Phe-NH₂).

This compound was synthesized as described in Method 1, except that 2-thiophenesulfonyl chloride (365.3 mg, 2 mmol) was used in place of 1-naphthalenesulfonyl chloride.

Data: ESMS 467(MH⁺); ¹H NMR (CD₃OD) d 1.282 (t, J=7.3 Hz, 1H), 1.35 (m, 2H), 1.37 (m, 2H), 2.91 (m, 1H), 2.99 (t, J=7.0 Hz, 2H), 3.08-3.31 (m, 2H), 3.73 (t, J=59 Hz 1H), 4.44 (t, J=5.5 Hz, 1H), 7.01 (t, 3.8 Hz, 1H), 7.20-2.28 (m, 6H), 7.47 (q, J=2.5, 1.2 Hz, 1H), 7.69 (q, J 3.7, 1.2 Hz, 1H); [a]_{D}=−5.9 (c=1% W/V in methanol);

HPLC Primesphere C-18 reverse phase column, 4.6×250 mm, 10-56% acetonitrile (0.1% TFA) in water (0.1% TFA) over 24 14.9 min;

Compound 1014B

N1-[(1S)-2-Amino-1-benzyl-2-oxoethyl]-(25)-(5-guanidino)-2-[(2-naphthylsulfonyl)amino]pentanamide (15). (Alternate name: 2-naphthalenesulfonylamido-Arg-Phe-NH₂).

This compound was synthesized as described in Method 1, except that 2-naphthalenesulfonyl chloride (453.36 mg, 2 mmol) was used in place of 1-naphthalenesulfonyl chloride.

Data: ESMS 511(MH⁺); ¹H NMR (CD₃OD) d 1.28 (t, J=7.3 Hz, 1H), 1.37 (m, 2H), 1.52 (m, 2H), 2.48 (q, J=8.3, 8.4 Hz, 1H), 2.86 (t, J=6.6 Hz, 1H), 2.93 (m, 2H), 3.10 (q, J=7 Hz, 1H), 3.69 (q, J=6.2, 1.4 Hz, 1H), 4.25 (q, J=6.7, 1.5 Hz, 1H), 7.01 (m, 2H), 7.16 (m, 3H), 7.63 (m, 2H), 7.7 (d, J=6.8, 1.8 Hz, 1H), 7.98 (m, 3H), 8.39 (s, 1H); ¹³C NMR (CD₃OD) d 25.00, 30.63, 38.01, 40.93, 54.90, 56.69, 56.72, 122.29, 127.08, 127.22, 127.34, 128.67, 129.46, 130.99, 131.06, 131.05, 132.78, 132.85, 132.91, 137.96, 142.92, 148.77, 157.79, 171.71, 174.82;

Anal. C₂₅H₃₀N₆O₄S+1.25 CF.COOH calcd. C, 50.57%; H, 4.82%; N, 12.87%; S, 4.91%; found C, 50.74%; H, 4.98%; N, 12.79%; S, 4.76%; [a]_{D}=−9.2 (c=1% W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.0 min;

Compound 1015B

N1-[(1S)-2-Amino-1-benzyl-2-oxoethyl]-(25)-{[amino(imino)methyl]amino}-2-[3,4-dimethoxyphenyl)sulfonyl]amino}pentanamide.

(Alternate name: 3,4-Dimethoxyphenylsulfonylamido-Arg-Phe-NH₂).

This compound was synthesized as described in Method 1, except that 3,4-dimethoxyphenylsulfonyl chloride (473.36 mg, 2 mmol) was used in place of 1-naphthalenesulfonyl chloride.

Data: ESMS 521(MH⁺); ¹H NMR (CD₃OD) d 1.26 (m, 2H), 1.46 (m, 2H), 2.72 (dd, J=8.5, 5.3 Hz, 1H), 3.00 (t, J=8 Hz, 2H), 3.06 (m, 2H), 3.59 (q, J=1.3, 6.1 Hz, 1H), 3.83 (s, 3H), 3.85 (s, 3H), 4.4 (q, J=2.3, 6.2 Hz, 1H), 6.96 (d, J=8.4 Hz, 1H), 7.15-7.3 (m, 5H), 7.3 (m, 1H), 7.37 (dd, J=6.4, 2.0 Hz, 1H); Anal. C₂₃H₃₂N₆O₆S+1.2 CF₃COOH calcd. C, 46.40%; H, 5.09%; N, 12.78%; S, 5.05%; found C, 46.62%; H, 4.98%; N, 12.91%; S, 4.86%; [a]_{D}=−5.3 (c=1% W/V in methanol);

HPLC Primesphere C-18 reverse phase column, 4.6×250 mm, 10 56% acetonitrile (0.1% TFA) in water (0.1% TFA) over 24 min, flow rate 1 mL/min, detection at 220 nm, retention time 14.9 min;

Compound 1016B

N1-[(1S)-2-Amino-1-benzyl-2-oxoethyl]-(2S)-([amino(imino)methyl]amino}-2-[4-chloro-3-nitrophenyl)sulfonyl]amino}pentanamide. (Alternate name: 4-Chloro-3-nitrophenylsulfonylamido-Arg-Phe-NH₂).

This compound was synthesized as described in Method 1, except that 4-chloro-3-nitrophenylsulfonyl chloride (512.14 mg, 2 mmol) was used in place of 1-naphthalenesulfonyl chloride.

Data: ESMS 540(MH⁺); ¹H NMR (CD₃OD) d 1.29 (t, J=7.3 Hz, 1H), 1.46-1.65 (m, 4H), 2.73 (dd, J=4.8, 8.6 Hz, 1H), 3.01 (dd, J 7, 8.7, 1H), 3.18 (m, 2H), 3.2 (q, J=6.2, 0.8 Hz, 1H), 4.3 (q, J=2.2, 6.3 Hz, 1H), 7.25 (m, 5H), 7.59 (d, J=8.6 Hz, 1H), 7.81 (dd, J=6.4, 1.2 Hz, 1H), 8.3 (m, 1H); Anal. C₂₁H₂₆ClN₇O₆S+1.5 CF₃COOH calcd. C, 40.54%; H, 3.90%; Cl, 4.99%; N, 13.79%; S, 4.51%; found C, 40.45%; H, 3.73%; Cl, 4.99%; N, 13.76%; S, 4.96%; [a]_{D}=+34.1 (c=1 W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 2002B

N1-[(1S)-2-Amino-1-benzyl-2-oxoethyl]-(2S)-[amino(imino)methyl]amino}-2-[2,4-dinitrophenyl)sulfonyl]amino}pentanamide. (Alternate name: 2, 4-Dinitrophenylsulfonylamido-Arg-phe-NH₂).

This compound was synthesized as described in Method 1, except that 2,4-dinitrophenylsulfonyl chloride (533.24 mg, 2 mmol) was used in place of 1-naphthalenesulfonyl chloride.

Data: ESMS 550.9(MH⁺); ¹H NMR (CD₃OD) d 1.29 (t, J=7.3 Hz, 1H), 1.41 (m, 2H), 1.59 (m, 2H), 2.75 (dd, J=4.4, 9.5 Hz, 1H), 3.00 (dd, J=5.3, 5.2 Hz, 1H), 3.18 (m, 2H) 4.03(q, J=2.3, 2.9 Hz, 1H), 4.25 (q, J=2.9, 3.0 Hz, 1H), 7.2 (m, 5H), 8.02 (d, J=4.0 Hz, 1H), 8.29 (dd, J=6.4, 2.2 Hz, 1H), 8.62 (d, J=2.2 Hz, 1H); Anal. C₂₁H₂₆N₈O₈S+1.275 CF₃COOH calcd. C, 40.65%; H, 3.95%; N, 16.10%; S, 4.61%; found C, 40.81%; H, 3.78%; N, 15.86%; S, 3.84%; [a]_{D}=−25.7 (c=1 W/V in methanol);

HPLC Primesphere C-18 reverse phase column, 4.6×250 mm, 10-56% acetonitrile (0.1% TFA) in water (0.1% TFA) over 24 min, flow rate 1 mL/min, detection at 220 nm, retention time 19.9 min;

Compound 1017B

N1-[(1S)-2-Amino-1-benzyl-2-oxoethyl]-(2S)-{[amino(imino)methyl]amino}-2-[(3-chloro-4-fluorophenyl)sulfonyl]amino}pentanamide.

(Alternate name: 3-Chloro-4-fluorophenylsulfonylamido-Arg-Phe-NH₂).

This compound was synthesized as described in Method 1, except that 3-chloro-4-fluorophenylsulfonyl chloride (458.12 mg, 2 mmol) was used in place of 1-naphthalenesulfonyl chloride.

Data: ESMS 513(MH⁺);

Compound 1018B

N1-[(1S)-2-Amino-1-benzyl-2-oxoethyl]-(25)-{[amino(imino)methyl]amino}-2-[(2-nitro-(4-trifluoromethyl)phenyl) sulfonyl]amino}pentanamide.

(Alternate name: 2-Nitro-4-trifluoromethyl phenylsulfonylamido-Arg-Phe-NH₂).

This compound was synthesized as described in Method 1, except that 2-Nitro-4-trifluoromethylphenylsulfonyl chloride (579.24 mg, 2 mmol) was used in place of 1-naphthalenesulfonyl chloride.

Data: ESMS 574(MH⁺); [a][_{D}=−32.9 (c=1% W/V in methanol);

Compound 1019B

N1-[(1S)-2-Amino-1-benzyl-2-oxoethyl]-(2S)-{[amino(imino)methyl]amino}-2-[(2,6-dichlorophenyl)sulfonyl]amino}pentanamide. (Alternate name: 2,6-Dichlorophenylsulfonylamido-Arg-Phe-NH₂).

This compound was synthesized as described in Method 1, except that 2,6-dichlorophenylsulfonyl chloride (491.02 mg, 2 mmol) was used in place of 1-naphthalenesulfonyl chloride.

Data: ESMS 529 (MH⁺); [a]_{D}=−5.9 (c=1% W/V in methanol);

Compound 1020B

N1-[(1S)-2-Amino-1-benzyl-2-oxoethyl]-(2S)-[amino(imino)methyl]amino}-2-[3-(2,5-dichlorothiophene)sulfonyl]amino}pentanamide. (Alternate name: 3-(2,5-Dichlorothiophene) sulfonylamido-Arg-Phe-NH₂).

This compound was synthesized as described in Method 1, except that 3-(2,5-dichlorothiophene) sulfonyl chloride (503.08 mg, 2 mmol) was used in place of 1-naphthalenesulfonyl chloride.

Data: ESMS 535, 536 (MH⁺); [a]_{D}=+1.9 (c=1% W/V in methanol);

Compound 2003B

N1-[(1S)-2-Amino-1-benzy1-2-oxoethyl1]-(2S)-{[amino(imino)methyl]amino}-2-[(3-methyl-6-methoxyphenyl) sulfonyl]amino]pentanamide. (Alternate name: 3-Methyl-6-methoxyphenylsulfonylamido-Arg-Phe-NH₂)

This compound was synthesized as described in Method 1, except that 3-methyl-6-methoxyphenylsulfonyl chloride (441.36 mg, 2 mmol) was used in place of 1-naphthalenesulfonyl chloride.

Data: ESMS 505 (MH⁺); [a]_{D}=−1.6 (c=1% W/V in methanol);

Compound 1021B

N1-[(1S)-2-Amino-1-benzy1-2-oxoethyl1]-(2S)-{[amino(imino)methyl]amino}-2-[(2,5-dichlorophenyl)sulfonyl]amino}pentanamide. (Alternate name: 2,5-Dichlorophenylsulfonylamido-Arg-Phe-NH₂).

This compound was synthesized as described in Method 1, except that 2,5-dichlorophenylsulfonyl chloride (491.02 mg, 2 mmol) was used in place of 1-naphthalenesulfonyl chloride.

Data: ESMS 529, 530(MH⁺); [a]_{D}=−0.3 (c=1% W/V in methanol);

Compound 1022B

N1-[(1S)-2-Amino-1-benzyl-2-oxoethyl]-(2S)-{[amino(imino)methyl]amino}-2-[3,4-dichlorophenyl)sulfonyl]amino}pentanamide.

This compound was synthesized as described in Method 1, except that 3,4-dichlorophenylsulfonyl chloride (491.02 mg, 2 mmol) was used in place of 1-naphthalenesulfonyl chloride.

Data: ESMS 528(MH⁺); [a]_{D}=+12.9 (c=1% W/V in methanol);

Compound 1023B

N1-[(1S)-2-Amino-1-benzyl-2-oxoethyl]-(2S)-{[amino(imino)methyl]amino}-2-[3-cyanophenyl)sulfonyl]amino}pentanamide.

(Alternate name: 3-Cyanophenylsulfonylamido-Arg-Phe-NH₂).

This compound was synthesized as described in Method 1, except that 3-cyanophenylsulfonyl chloride (403.26 mg, 2 mmol) was used in place of 1-naphthalenesulfonyl chloride.

Data: ESMS 486(MH⁺); [a]_{D}=+14.9 (c=1% W/V in methanol);

Compound 1024B

N1-[(1S)-2-Amino-1-benzyl-2-oxoethyl]-(2S)-{[amino(imino)methyl]amino}-2-[pentafluorophenyl)sulfonyl]amino}pentanamide.

(Alternate name: Pentafluorophenylsulfonylamido-Arg-Phe-NH₂).

This compound was synthesized as described in Method 1, except that pentafluorophenylsulfonyl chloride (533.14 mg, 2 mmol) was used in place of 1-naphthalenesulfonyl chloride.

Data: ESMS 550(MH⁺); [a]_{D}=+25.1(c=1% W/V in methanol);

Compound 1025B

N1-[(1S)-2-Amino-1-benzyl-2-oxoethyl]-(2S)-{[amino(imino)methyl]amino}-2-[5-bromo-2-methoxyphenyl)sulfonyl]amino}pentanamide. (Alternate name: 5-Bromo-4-methoxyphenylsulfonylamido-Arg-Phe-NH₂).

This compound was synthesized as described in Method 1, except that 5-bromo-4-methoxyphenylsulfonyl chloride (571.10 mg, 2 mmol) was used in place of 1-naphthalenesulfonyl chloride.

Data: ESMS 569(MH⁺); [a]_{D}=+7.9 (c=1% W/V in methanol);

Compound 1026B

N1-[(1S)-2-Amino-1-benzyl-2-oxoethyl]-(2S)-{[amino(imino)methyl]amino}-2-[2-nitrophenyl)sulfonyl]amino}pentanamide. (Alternate name: 2-Nitrophenylsulfonylamido-Arg-Phe-NH₂).

This compound was synthesized as described in Method 1, except that 2-nitrophenylsulfonyl chloride (443.24 mg, 2 mmol) was used in place of 1-naphthalenesulfonyl chloride.

Data: ESMS 506(MH⁺); [a]_{D}=−38.1 (c=1% W/V in methanol);

Compound 1027B

N1-[(1S)-2-Amino-1-benzyl-2-oxoethyl]-(2S)-{[amino(imino)methyl]amino}-2-[2-cyanophenyl)sulfonyl]amino}pentanamide.

(Alternate name: 2-Cyanophenylsulfonylamido-Arg-Phe-NH₂).

This compound was synthesized as described in Method 1, except that 2-cyanophenylsulfonyl chloride (403.26 mg, 2 mmol) was used in place of 1-naphthalenesulfonyl chloride.

Data: ESMS 486(MH⁺); ¹H NMR (CD₃OD) d 1.6 (m, b, 4H), 2.75 (dd, J 4.4, 9.5 Hz, 1H), 3.00 (dd, J 5.3, 5.2 Hz, 1H) 3.12 (m, 2H), 3.9(m, 1H), 4.32 (m, 1H), 7.25 (m, 5H), 7.62 (m, 1H), 7.9 (m 1H)

Compound 1028B

N1-[(1S)-2-Amino-1-benzyl-2-oxoethyl]-(2R)-{[amino(imino)methyl]amino}-2-[4-fluorophenyl)sulfonyl]amino}pentanamide. (Alternate name: 4-Fluorophenylsulfonylamido-(D)Arg-Phe-NH₂).

This compound was synthesized as described in Method 1, except that (D)Arginine(Pbf) was used in place of (L)Arginine(Pbf), and 4-fluorophenylsulfonyl chloride (389.22 mg, 2 mmol) was used in place of 1-naphthalenesulfonyl chloride.

Data: ESMS 479(MH⁺);

Compound 1029B

N1-[(1R)-2-Amino-1-benzyl-2-oxoethyl]-(2S)-{[amino(imino)methyl]amino}-2-[2-naphthalene)sulfonyl]amino}pentanamide. (Alternate name: 2-Naphthalenesulfonylamido-Arg-(D)Phe-NH₂).

This compound was synthesized as described in Method 1, except that (D)Phenylalanine was used in place of (L)Phenylalanine, and 2-naphthalenesulfonyl chloride (453.36 mg, 2 mmol) was used in place of 1-naphthalenesulfonyl chloride.

Data: ESMS 510(MH⁺);

Compound 1030B

N1-[(1S)-2-Amino-1-benzyl-2-oxoethyl]-(2R)-{[amino(imino)methyl]amino}-2-2-bromophenyl) sulfonyl]amino}pentanamide. (Alternate name: 2-Bromophenylsulfonylamido-(D)Arg-Phe-NH₂).

This compound was synthesized as described in Method 1, except that (D)Arginine(Pbf) was used to substitute (L) Arginine (Pbf), and 2-bromophenylsulfonyl chloride (511.04 mg, 2 mmol) was used in place of 1-naphthalenesulfonyl chloride.

Data: ESMS 540(MH⁺);

Compound 3001B

N1-[(1S)-2-Amino-1-benzyl-2-oxoethyl]-(2R)-{[amino(imino)methyl]amino}-2-[1-naphthalene) sulfonyl]amino}pentanamide. (Alternate name 1-Naphthalenesulfonylamido-(D)Arg-Phe-NH₂).

This compound was synthesized as described in Method 1, except that (D)Arginine(Pbf) was used in place of (L)Arginine(Pbf).

Data: ESMS 511(MH⁺);

Compound 1031B

N1-[(1R)-2-Amino-1-benzyl-2-oxoethyl]-(2S)-{[amino(imino)methyl]amino}-2-[2-bromophenyl) sulfonyl]amino}pentanamide. (Alternate name: 2-Bromophenylsulfonylamido-Arg-(D)Phe-NH₂).

This compound was synthesized as described in Method 1, except that (D)Phenylalanine was used to substitute (L)Phenylalanine, and 2-bromophenylsulfonyl chloride (511.04 mg, 2 mmol) was used in place of 1-naphthalenesulfonyl chloride.

Data: ESMS 540(MH⁺);

Compound 1032B

N1-[(1R)-2-Amino-1-benzyl-2-oxoethyl]-(2S)-{[amino(imino)methyl]amino}-2-[2, 6-difluorophenyl)sulfonyl]amino]pentanamide. (Alternate name: 2,6-Difluorophenylsulfonylamido-Arg-(D)Phe-NH₂).

This compound was synthesized as described in Method 1, except that (D)Phenylalanine was used to substitute (L)Phenylalanine, and 2,6-difluorophenylsulfonyl chloride (425.20 mg, 2 mmol) was used in place of 1-naphthalenesulfonyl chloride.

Data: ESMS 511(MH⁺);

Compound 1033B

N1-[(1R)-2-Amino-1-benzyl-2-oxoethyl]-(2S)-{[amino(imino)methyl]amino}-2-[4-fluorophenyl)sulfonyl]amino}pentanamide. (Alternate name: 4-Fluorophenylsulfonylamido-Arg-(D)Phe-NH₂).

This compound was synthesized as described in Method 1, except that (D)Phenylalanine was used to substitute (L)Phenylalanine, and 4-fluorophenylsulfonyl chloride (389.22 mg, 2 mmol) was used in place of 1-naphthalenesulfonyl chloride.

Table 2. Summary of compounds prepared in Part B.

TABLE 2
Summary of compounds prepared in Part B.

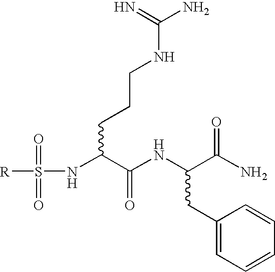

| | | Amino Acid |
|---|---|---|
| Compound | R-group | Chirality |
| 1001B | 1-naphthalene- | Both (L) |
| 1002B | 3-nitrobenzene | Both (L) |
| 1003B | 4-nitrobenzene- | Both (L) |
| 1004B | 2,6-difluorobenzene- | Both (L) |
| 1005B | 4-fluorobenzene- | Both (L) |
| 1006B | 4-chlorobenzene- | Both (L) |
| 2001B | 2-bromobenzene- | Both (L) |
| 1007B | p-tolyl- | Both (L) |
| 1008B | phenyl- | Both (L) |
| 1009B | 4-methoxybenzene | Both (L) |
| 1010B | 2,4-dichlorobenzene- | Both (L) |
| 1011B | α-toluene- | Both (L) |
| 1012B | 4-iodobenzene- | Both (L) |
| 1013B | 2-thiophene- | Both (L) |
| 1014B | 2-naphthalene | Both (L) |
| 1015B | 3,4-dimethoxybenzene- | Both (L) |
| 1016B | 4-chloro-3- | |
| | nitrobenzene | |
| 2002B | 2,4-dinitrobenzene- | Both (L) |
| 1017B | 3-chloro-4- | Both (L) |
| | fluorobenzene- | |
| 1018B | 2-nitro-4- | Both (L) |
| | trifluoromethylbenzene | |
| 1019B | 2,6-dichlorobenzene | Both (L) |
| 1020B | 3-(2,5- | Both (L) |
| | dichlorothiophene)- | |
| 2003B | 2-methoxy-4- | Both (L) |
| | methylbenzene- | |
| 1021B | 2,5-dichlorobenzene- | Both (L) |
| 1022B | 3,4-dichlorobenzene- | Both (L) |
| 1023B | 3-cyanobenzene- | Both (L) |
| 1024B | pentafluorobenzene- | Both (L) |
| 1025B | 5-bromo-2- | Both (L) |
| | methoxybenzene- | |
| 1026B | 2-nitrobenzene- | Both (L) |
| 1027B | 2-cyanobenzene- | Both (L) |
| 1028B | 4-fluorophenyl- | (D) Arg, (L) Phe |
| 1029B | 2-naphthalene- | (L) Arg, (D) Phe |
| 1030B | 2-bromophenyl- | (D) Arg, (L) Phe |
| 3001B | 1-naphthalene- | (D) Arg, (L) Phe |
| 1031B | 2-bromophenyl- | (L) Arg, (D) Phe |
| 1032B | 2,6-difluorophenyl- | (L) Arg, (D) Phe |
| 1033B | 4-fluorophenyl- | (L) Arg, (D) Phe |

III. Testing of Chemical Compounds at NPFF Receptors

The binding properties of compounds were evaluated at cloned NPFF receptors using protocols described herein and in PCT International Publication No. WO 00/18438, the disclosure of which is hereby incorporated by reference in its entirety into this application.

The binding data reflect competitive displacement of ([¹²⁵I] 1DMeNPFF).

Compounds were tested at concentrations ranging from 0.001 nM to 3600 nM, unless otherwise noted.

Activity of the compounds of the present invention was measured at cloned NPFF receptors according to functional assays as previously described by Bonini, J. A. et al. (2000). Agonist potency (EC₅₀) is the concentration of a compound required to elicit 50% of maximum response. Intrinsic activity of a compound is measured as the percent of maximum response elicited by the ligand, neuropeptide FF.

Results are presented in Tables 3-7.

In one series, one or both of the Arginine or Phenylalanine residues were changed to their corresponding D-isomer. This modification is expected to further improve the stability of these compounds against enzymatic degradation. Binding and functional activities of these compounds at rat NPFF1 and NPFF2 receptors are shown in Table 5.

Table 8 shows the cross-reactivity of NPFP compounds. The binding affinity (Ki) of these compounds were tested according to the protocols described herein at the following receptors; human α_{1A}, α_{1B}, α_{1D}, α_{2A}, α_{2B}, and α_{2C} adrenergic receptors; human Y1, Y2, Y4, and Y5 receptors; and N-Methyl-D-aspartic acid (NMDA) receptor channels. The binding interactions of these compounds were additionally tested at the norepinephrine (NE) transporter (NE uptake) and serotonin (5-hydroxytryptamine (5HT)) transporter (5HT uptake) according to protocols described herein

TABLE 3
Binding affinities at Recombinant Human and
Rat NPFF Receptor Subtypes NPFF1 and NPFF2

| | hNPFF1 | hNPFF2 | rNPFF1 | rNPFF2 |
|---|---|---|---|---|
| Compound | Ki (nM) | Ki (nM) | Ki (nM) | Ki (nM) |
| 3001A | 46 | 1717 | 50 | 1222 |
| 1001A | 240 | 2043 | 202 | >10,000 |
| 1007A | 53 | 260 | 146 | 699 |
| 6001A | 23 | 374 | 11 | 433 |
| 4006A | 13 | 91 | 7 | 185 |
| 6003A | 28 | 113 | 21 | 203 |
| 6002A | 157 | 952 | 91 | 883 |
| 4005A | 24 | 123 | 25 | 282 |
| 4009A | 144 | 826 | 153 | 871 |
| 4004A | 113 | 1,214 | 153 | 2584 |
| 4008A | 82 | 514 | 64 | 882 |
| 4001A | 21 | 150 | 30 | 556 |
| 4003A | 207 | 2,125 | 176 | 1,252 |
| 1020A | NT | NT | 18 | 273 |
| 4007A | NT | NT | 44 | 619 |
| 1002A | NT | NT | 134 | 3,919 |
| 1019A | NT | NT | 57 | 2,874 |
| 1014A | NT | NT | 300 | 3,439 |
| 1026A | NT | NT | 802 | >10,000 |
| 1036A | NT | NT | 132 | 2458 |
| 1013A | NT | NT | 332 | 2019 |
| 1011A | NT | NT | 201 | >10,000 |
| 1021A | NT | NT | 56 | 881 |
| 1030A | NT | NT | 176 | 4,864 |
| 2001A | 50 | 376 | 8 | 221 |
| 1015A | NT | NT | 42 | 1,108 |
| 1035A | NT | NT | 842 | 1,183 |
| 1003A | NT | NT | 238 | 1,638 |
| 2002A | NT | NT | 77 | 461 |
| 1039A | NT | NT | 68 | 2,930 |
| 4002A | 50 | 232 | 11 | 308 |
| 1012A | NT | NT | 733 | 4845 |
| 1028A | NT | NT | 386 | 817 |
| 1032A | NT | NT | 291 | 1638 |
| 1029A | NT | NT | 912 | 1201 |
| 1031A | NT | NT | 794 | 3223 |
| 1033A | NT | NT | 481 | 5864 |
| 1004A | NT | NT | 710 | 1488 |
| 1016A | NT | NT | 565 | 2,496 |
| 1024A | NT | NT | 659 | 5,593 |
| 1018A | NT | NT | 303 | 1299 |
| 1022A | NT | NT | 126 | 602 |
| 1017A | NT | NT | 234 | 5919 |
| 1037A | NT | NT | 143 | 824 |
| 1008A | NT | NT | 155 | 1121 |
| 1038A | NT | NT | 95 | 602 |
| 1005A | NT | NT | 316 | 2138 |
| 2004A | NT | NT | 392 | 262 |
| 2003A | NT | NT | 371 | 195 |
| 2005A | NT | NT | 88 | 268 |
| 1006A | NT | NT | 410 | 1071 |
| 1010A | NT | NT | 311 | 3480 |
| 1009A | NT | NT | 312 | 703 |
| 2006A | NT | NT | 788 | 3674 |
| 5002A | 40 | 460 | 30 | 569 |
| 5003A | 152 | 1172 | 532 | 4423 |
| 1034A | NT | NT | 82 | 1537 |
| 5001A | NT | NT | 24 | 115 |
| 1023A | 228 | 2919 | 4 | 1019 |
| 1025A | NT | NT | 253 | 4534 |
| 1027A | NT | NT | 606 | 3154 |

NT = Not Tested

TABLE 4
Binding and Functional Activities of Compounds
at Rat NPFF Receptor Subtypes NPFF1 and NPFF2

| | Ki Values | | Functional Activity | | | |
|---|---|---|---|---|---|---|
| | (nM) | | rNPFF1 | rNPFF1 | rNPFF2 | rNPFF2 |
| Compound | rNPFF1 | rNPFF2 | EC₅₀ (nM) | I.A. % | EC₅₀ (nM) | I.A. % |
| 1001B | 261 | 1447 | 38 | 88 | 527 | 81 |
| 1002B | 136 | 1254 | 139 | 88 | 846 | 89 |
| 1003B | 732 | 2609 | 149 | 74 | 1871 | 44 |
| 1004B | 173 | 1447 | 117 | 79 | >3160 | 43 |
| 1005B | 150 | 1366 | 104 | 71 | 3496 | 46 |
| 1006B | 266 | 1014 | 151 | 75 | 3725 | 43 |
| 2001B | 112 | 2982 | 679 | 81 | >3160 | 9 |
| 1007B | 756 | 3083 | 286 | 79 | 2295 | 55 |
| 1008B | 321 | 4409 | 4698 | 70 | 5621 | 20 |
| 1009B | 321 | 1086 | Nd | Nd | Nd | Nd |
| 1010B | 871 | 1862 | 594 | 85 | 2980 | 28 |
| 1011B | 5959 | >10000 | 765 | 74 | 1342 | 62 |
| 1012B | 1427 | 2920 | 358 | 71 | 1418 | 93 |
| 1013B | 211 | 6393 | 135 | 80 | >3160 | 42 |
| 1014B | 314 | 2784 | 52 | 74 | 906 | 73 |
| 1015B | 462 | >10000 | 140 | 84 | 1815 | 74 |
| 1016B | 151 | 2090 | 62 | 72 | 660 | 81 |
| 2002B | 1387 | 5489 | 3160 | 34 | >10000 | 5 |
| 1017B | 1136 | 3564 | 376 | 84 | >3160 | 45 |
| 1018B | 1949 | 4430 | >3160 | 21 | 5621 | 10 |
| 1019B | 815 | 3375 | 2196 | 56 | >3160 | 45 |
| 1020B | 1954 | 5152 | >10000 | 1 | >10000 | 2 |
| 2003B | 2181 | >10000 | 461 | 102 | >3160 | 52 |
| 1021B | 335 | 2031 | 1027 | 78 | 2330 | 59 |
| 1022B | Nd | Nd | 1863 | 86 | >3160 | 34 |
| 1023B | 496 | 9919 | 166 | 90 | >3160 | 28 |
| 1024B | 486 | 5396 | 720 | 69 | >3160 | 43 |
| 1025B | 328 | 4122 | 596 | 78 | >3160 | 33 |
| 1026B | 535 | 3498 | 412 | 79 | >3160 | 59 |
| 1027B | 515 | 6171 | 183 | 52 | >3160 | 48 |

Nd = Not Determined

TABLE 5
Binding and Functional Activities of D-Arg- or D-Phe-
Containing Compounds at Rat NPFF1 and NPFF2 Receptors

| | Ki Values | | Functional Activity | | | |
|---|---|---|---|---|---|---|
| | (nM) | | rNPFF1 | rNPFF1 | rNPFF2 | rNPFF2 |
| Compound | rNPFF1 | rNPFF2 | EC₅₀ (nM) | I.A. % | EC₅₀ (nM) | I.A. % |
| 1028B | 1285 | 8056 | 404 | 46 | 1583 | 62 |
| 1029B | 399 | 2689 | 477 | 30 | >3160 | 86 |
| 1030B | 251 | 6200 | 655 | 35 | 1641 | 71 |
| 3001B | 46 | 2863 | >10000 | 1 | 378 | 79 |
| 1031B | 2574 | 6029 | 856 | 32 | 1574 | 24 |
| 1032B | 1289 | >10000 | 644 | 47 | 2758 | 61 |
| 1033B | 458 | >10000 | 1597 | 42 | 1941 | 57 |

TABLE 6
Agonist Potency (EC50) and Intrinsic Activity (IA)
at Recombinant Human Neuropeptide FF Receptors

| | hNPFF1 | hNPFF1 | hNPFF2 | hNPFF2 |
|---|---|---|---|---|
| | EC50 | IA | EC50 | IA |
| Compound | (nM) | (% NPFF) | (nM) | (% NPFF) |
| 3001A | >10,000 | Inactive | >10,000 | Inactive |
| 6001A | >10,000 | Inactive | >10,000 | Inactive |
| 4006A | >10,000 | Inactive | >10,000 | Inactive |
| 2001A | 3453 | Inactive | 625 | 84% |
| 4002A | >10,000 | Inactive | 314 | 69% |
| 5002A | >10,000 | Inactive | 1707 | 75% |
| 5003A | >10,000 | Inactive | 3160 | 45% |
| 1023A | >10,000 | Inactive | 4114 | 43% |

NT = Not Tested

TABLE 7
Agonist Potency (EC50) and Intrinsic Activity (IA)
at Recombinant Rat Neuropeptide FF Receptors

| | rNPFF1 | rNPFF1 | rNPFF2 | rNPFF2 |
|---|---|---|---|---|
| | EC50 | IA | EC50 | IA |
| Compound | (nM) | (% NPFF) | (nM) | (% NPFF) |
| 3001A | NT | NT | NT | NT |
| 1001A | >10,000 | Inactive | 3084 | 16% |
| 1007A | >10,000 | Inactive | 1296 | 66% |
| 6001A | >10,000 | Inactive | >10,000 | Inactive |
| 4006A | >10,000 | Inactive | 269 | 32% |
| 6003A | >10,000 | Inactive | >10,000 | Inactive |
| 6002A | >10,000 | Inactive | >10,000 | Inactive |
| 4005A | >10,000 | Inactive | 389 | 61% |
| 4009A | >10,000 | Inactive | 3160 | 70% |
| 4004A | >10,000 | Inactive | 1528 | 65% |
| 4008A | >10,000 | Inactive | 411 | 65% |
| 4001A | >10,000 | Inactive | 404 | 68% |
| 4003A | >10,000 | Inactive | 3160 | 26% |
| 1020A | >10,000 | Inactive | 695 | 90% |
| 4007A | >10,000 | Inactive | 2637 | 17% |
| 1002A | >10,000 | Inactive | 5621 | 24% |
| 1019A | >10,000 | Inactive | 2543 | 31% |
| 1014A | >10,000 | Inactive | 2462 | 47% |
| 1026A | >10,000 | Inactive | >10,000 | 19% |
| 1036A | >10,000 | Inactive | 369 | 78% |
| 1013A | >10,000 | Inactive | 690 | 52% |
| 1011A | >10,000 | Inactive | >10,000 | Inactive |
| 1021A | >10,000 | Inactive | 283 | 76% |
| 1030A | >10,000 | Inactive | 625 | 85% |
| 2001A | 242 | 71% | 97 | 103% |
| 1015A | >10,000 | Inactive | 272 | 56% |
| 1035A | >10,000 | Inactive | 3160 | 52% |
| 1003A | >10,000 | Inactive | 392 | 83% |
| 2002A | 250 | 51% | 423 | 92% |
| 1039A | >10,000 | Inactive | 272 | 78% |
| 4002A | >10,000 | Inactive | 125 | 84% |
| 1012A | >10,000 | Inactive | 1616 | 80% |
| 1028A | >10,000 | Inactive | 758 | 79% |
| 1032A | 374 | 31% | 459 | 93% |
| 1029A | >10,000 | 28% | 2046 | 31% |
| 1031A | >10,000 | Inactive | 2187 | 66% |
| 1033A | >10,000 | Inactive | 3160 | 51% |
| 1004A | 1469 | 36% | 440 | 90% |
| 1016A | >10,000 | Inactive | 3160 | 74% |
| 1024A | >10,000 | Inactive | >10,000 | Inactive |
| 1018A | >10,000 | Inactive | >10,000 | Inactive |
| 1022A | 3160 | 19% | 190 | 81% |
| 1017A | >10,000 | Inactive | >10,000 | 23% |
| 1037A | >10,000 | Inactive | 3160 | 71% |
| 1008A | >10,000 | Inactive | 619 | 85% |
| 1038A | >10,000 | Inactive | 48 | 74% |
| 1005A | >10,000 | Inactive | 3160 | 21% |
| 2004A | 194 | 40% | 124 | 101% |
| 2003A | 171 | 56% | 49 | 89% |
| 2005A | 137 | 56% | 105 | 81% |
| 1006A | >10,000 | 15% | 1080 | 22% |
| 1010A | >10,000 | Inactive | >10,000 | 22% |
| 1009A | 1494 | Inactive | 5621 | 22% |
| 2006A | 886 | 38% | 1953 | 47% |
| 5002A | 157 | 41% | 259 | 90% |
| 5003A | 440 | 27% | 9993 | 57% |
| 1034A | 610 | 63% | 394 | 101% |
| 5001A | 123 | 28% | 69 | 82% |
| 1023A | >10,000 | Inactive | 3160 | 35% |
| 1025A | >10,000 | Inactive | 3160 | 27% |
| 1027A | >10,000 | Inactive | >10,000 | 31% |

NT = Not Tested

TABLE 8
Cross-Reactivity of NPFF Compounds at Different Receptors

| | NMDA | NE Uptake | 5HT Uptake | hα1A | hα1B | hα1D | hα2A | hα2B | hα2C |
|---|---|---|---|---|---|---|---|---|---|
| Compound | Ki(nM) | Ki(nM) | Ki(nM) | Ki(nM) | Ki(nM) | Ki(nM) | Ki(nM) | Ki(nM) | Ki(nM) |
| 3001A | | 21,442 | 22,601 | | | | | | |
| 1001A | | | | 5,033 | 9,857 | 12,918 | | | |
| 1007A | | 1,940 | 26 | 3,271 | 3,044 | 8,579 | | | |
| 6001A | | 12,713 | 10,453 | | | | | | |
| 4006A | | 12.359 | 8.793 | | | | | | |
| 4005A | 21.685 | 8.287 | 2.577 | | | | 199 | 3.754 | 765 |
| 1020A | 9.400 | 8.021 | 5,587 | | | | 1.168 | 20,871 | 4.129 |
| 1013A | 23.264 | 41.195 | 2,022 | | | | | | |
| 2001A | 8,134 | 1,156 | 962 | | | | 808 | 7,410 | 1.912 |
| 1003A | 19,503 | | | | | | 1,974 | 31,751 | 8,455 |
| 2002A | 7,622 | | | | | | 713 | 12,906 | 3,331 |
| 4002A | 12,806 | | | | | | 267 | 10,380 | 1,399 |

| | hY1 | hY2 | hY4 | hY5 | hNPFF1 | hNPFF2 | rNPFF1 | rNPFF2 |
|---|---|---|---|---|---|---|---|---|
| Compound | Ki(nM) | Ki(nM) | Ki(nM) | Ki(nM) | Ki(nM) | Ki(nM) | Ki(nM) | Ki(nM) |
| 3001A | | | | | 46 | 1,717 | 50 | 1,222 |
| 1001A | | | | | 240 | 2,043 | 202 | >10,000 |
| 1007A | 5.239 | >50000 | 2.613 | 74 | 53 | 260 | 146 | 699 |
| 6001A | >50000 | >50000 | 14,269 | 742 | 23 | 374 | 11 | 433 |
| 4006A | | | | | 13 | 91 | 7 | 185 |
| 4005A | 12.253 | >50000 | 5.047 | 358 | 24 | 123 | 25 | 282 |
| 1020A | | | | | NT | NT | 18 | 273 |
| 1013A | | | | | NT | NT | 332 | 2,019 |
| 2001A | | | | | 50 | 376 | 8 | 221 |
| 1003A | | | | | NT | NT | 238 | 1,638 |
| 2002A | | | | | NT | NT | 77 | 461 |
| 4002A | | | | | 50 | 232 | 11 | 308 |

IV. In vivo Testing of Compounds

The effects of NPFF selective compounds on the micturition reflex were assessed in the “distension-induced rhythmic contraction” (DIRC) model (also called “volume-induced reflex contraction” model) in rats, as described in previous publications (e.g. Maggi et al, 1987; Morikawa et al, 1992; Guarneri et al, 1993, the contents of which are incorporated by reference into the subject application). This model is widely considered to be predictive for the actions of drugs to treat human urge incontinence (also refered to as detrusor instability or unstable bladder). Examples of drugs that are active in this model which also are used therapeutically in humans include oxybutynin and baclofen (Morikawa et al, 1992); imipramine and nortriptyline (Pietra et al, 1990); and nifedipine and terodiline (Guarneri et al, 1993).

DIRC Model

Female Sprague Dawley rats weighing approximately 300 g were anesthetized with subcutaneous urethane (1.2 g/kg). The trachea was cannulated with PE240 tubing to provide a clear airway throughout the experiment. A midline abdominal incision was made and the left and right ureters were isolated. The ureters were ligated distally (to prevent escape of fluids from the bladder) and cannulated proximally with PE10 tubing. The incision was closed using 4-0 silk sutures, leaving the PE10 lines routed to the exterior for the elimination of urine. The bladder was canulated via the transurethral route using PE50 tubing inserted 2.5 cm beyond the urethral opening. This cannula was secured to the tail using tape and connected to a pressure transducer. To prevent leakage from the bladder, the cannula was tied tightly to the exterior urethral opening using 4-0 silk.

To initiate the micturition reflex, the bladder was first emptied by applying pressure to the lower abdomen, and then filled with normal saline in 100 μl increments (maximum=2 ml) until spontaneous bladder contractions occurred (typically 20-40 mmHg at a rate of one contraction every 2 to 3 minutes. Once a regular rhythm was established, vehicle (saline) or test compounds were administered i.v. to examine their effects on bladder activity. The effect of a compound which inhibited the micturition reflex was expressed as its “disappearance time”, defined as the time between successive bladder contractions in the presence of the test compound minus the time between contractions before compound administration.

Results

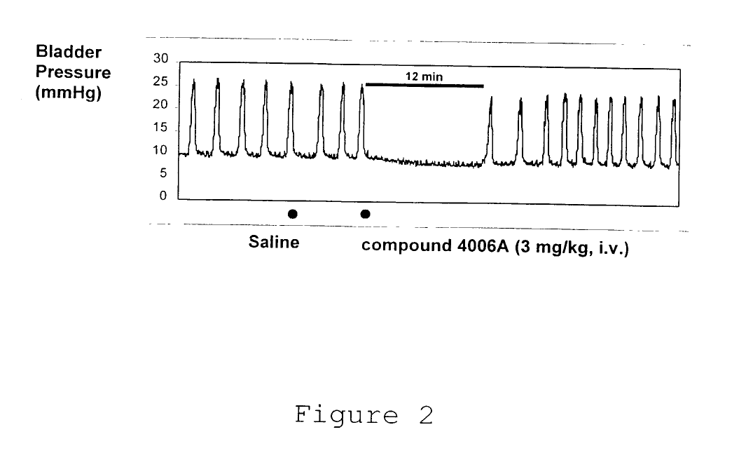

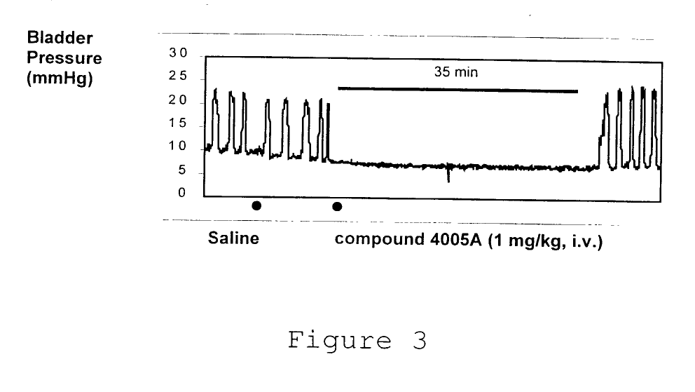

Compound 4005A at a dose of 1 mg/kg, i.v. produced complete inhibition of distention-induced contractions of the rat bladder, resulting in a disappearance time of 35 minutes (FIG. 3). Compound 4006A at a dose of 3 mg/kg, i.v. produced complete inhibition of distention induced contractions of the rat bladder, resulting in a disappearance time of 12 minutes (FIG. 2).

Discussion

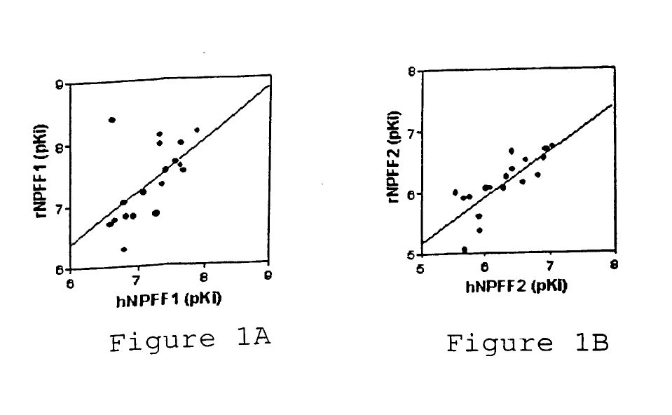

The correlation between binding affinities at human and rat recombinant neuropeptide FF (NPFF1 and NPFF2) receptors is shown in FIGS. 1A-1B. When comparing the binding affinities of compounds at the human and rat NPFF receptors, a positive correlation with slope values close to unity, the line of identity, is obtained. These data indicate that the binding affinity for a compound at the rat receptor will be predictive of its binding affinity at the human receptor.

The results presented herein represent the first demonstration that synthetic ligands which are active as agonists at the NPFF2 receptor inhibit the micturition reflex. In this regard their actions mimic the action of the endogenous peptide ligand NPFF. The ability of these compounds to inhibit the micturition reflex in this model can be taken as an indication that they will be effective in the treatment of urge incontinence in humans.

The compounds discussed herein can be classified as agonists and antagonists based on the following parameters: an agonist as a ligand has an intrinsic activity (IA)>15%, while an antagonist as a ligand has a Ki≦1.2 mM and an intrinsic activity (IA)≦15% at the rat cloned neuropeptide FF (NPFF) receptors.

Based on this definition the compounds can be classified as follows:

Compounds 2001A to 2006A, and 5001A to 5003A are quinolino-guanidines that are concurrently agonists at both the NPFF1 and NPFF2 receptors; compounds 1001B to 1008B, lOlOB to 1017B, 1019B, 1021B to 1033B, and 2003B are sulfonylamides that are concurrently agonists at both the NPFF1 and NPFF2 receptors;

Compounds 1001A to 1039A, and 4001A to 4009A are quinazolino-guanidines that are antagonists at the NPFF1 receptor and agonists at the NPFF2 receptor; compound 3001B is a sulfonylamide that is an antagonist at the NPFF1 receptor and an agonist at the NPFF2 receptor;

Compounds 3001 A, and 6001A to 6003A are quinolino-guanidines that are concurrently antagonists at both the NPFF1 and NPFF2 receptors.

Compounds that are agonists at the NPFF2 receptor are suitable for treating incontinence, and also pain.

Compounds that are concurrently agonists at both the NPFF1 and NPFF2 receptors are suitable for treating incontinence, and also pain.

Compounds that are concurrently antagonists at both the NPFF1 and NPFF2 receptors have a pro-opioid (analgesic) effect.

Compounds that are agonists at the NPFF1 receptor are suitable for treating obesity and eating disorders.

### REFERENCES

Allard, M., Labrouche, S., Nosjean, A., and Laguzzi, R. Mechanisms underlying the cardiovascular responses to peripheral administration of NPFF in the rat. J. Pharmacol. Exp. Ther. 274(1):577-583, 1995.

Allard, M., Zajac, J.M., and Simonnet, G. Autoradiographic distribution of receptors to FLFQPQRFamide, a morphine-modulating peptide, in rat central nervous system. Neuroscience 49(1):101-116, 1992.

Allard, M., Geoffre, S., Legendre, P., Vincent, J. D., and Simonnet, G. Characterization of rat spinal cord receptors to FLFQPQRFamide, a mammalian morphine modulating peptide: a binding study. Brain Res. 500(1-2):169-176, 1989.

Arima, H., Takashi, M., Kondo, K., Iwasaki, Y. and Oiso, Y. Centrally administered neuropeptide FF inhibits arginine vasopressin release in conscious rats. Endocrinology. 137 (5): 1523-1529, 1996.

Bonini J A, Jones K A, Adham N, Forray C, Artymyshyn R, Durkin M M, Smith K E, Tamm J A, Boteju L W, Lakhlani P P, Raddatz R, Yao W J, Ogozalek K L, Boyle N, Kouranova E V, Quan Y, Vaysse P J, Wetzel J M, Branchek T A, Gerald C, Borowsky B. Identification and characterization of two G protein-coupled receptors for neuropeptide FF. J. Biol. Chem. 275(50):39324-31, 2000.

Bourguignon, J. J.; Collot, V.; Didier, B.; Laulin, J. P. and Simmonet, G. Analogs of NPFF, a neuropeptide which modulates morphine analgesia: Proceedings of the XIVth International Symposium on Medicinal Chemistry, Awouters, F. (Ed.), 1997, Elsevier Science B. V., pp 35-44.

Brown, J. P., (1964) “Reactions of 2,2-Dialkyl-1,2-dihydroquinolines, Part I. Preparation of 2-Guanidinoquinazolines”, J. Chem. Soc. Pages 3012-3016.

Brussard, A. B.; Kits, A.; Ter Maat, A. H.; Mulder, A. H. and Schoffelmeer, A. N. M., Peptides 10, 735, 1989.

Coudore, M. A.; Courteix, C.; Eschalier, A.; Zajac, J.-M., Wilcox, G. L.; Fialip, J. Resumes de la lere Reunion de la Societe Francaise de Pharmacologie (Marseilles, France), vol. 17, p23, 1997.

Cowan, J. A., (1986) “Cu2+/BH4− Reduction System: Synthetic Utility And Mode of Action”, Tetrahedron Lett 27: 1205-1208.

Cullen, B. (1987). Use of eukaryotic expression technology in the functional analysis of cloned genes. Methods Enzymol. 152: 685-704.

Demichel, P., Rodrigues, J.C., Roquebert, J. and Simonnet, G. NPFF, a FMRF—NH2-like peptide, blocks opiate effects on ileum contractions. Peptides. 14: 1005-1009, 1993.

Devillers, J. P., Mazarguil, H., Allard, M., Dickenson, A. H., Zajac, J. M., and Simonnet, G. Characterization of a potent agonist for NPFF receptors: binding study on rat spinal cord membranes. Neuropharmacology 33(5):661-669, 1994.

Fehmann, H. C., McGreggor, G., Weber, V., Eissele, R., Goke, R., Doke, B. and Arnold, R. The effects of two FMRFamide related peptides (A-18-F-amide and F-8-F-amide; ‘morphine modulating peptides’) on the endocrine and exocrine rat pancreas. Neuropeptides. 17: 87-92, 1990.

Gicquel, S., Fioramonti, J., Bueno, L. and Zajac, J.-M. Effects of F8Famide analogs on intestinal transit in mice. Peptides. 14: 749-753, 1993.

Gouarderes, C., Tafani, J. A. M., and Zajac, J. M. Affinity of neuropeptide FF analogs to opioid receptors in the rat spinal cord. Peptides 19(4):727-730, 1998.

Gouarderes, C., Jhamandas, K., Sutak, M., and Zajac, J. M. Role of opioid receptors in the spinal antinociceptive effects of neuropeptide FF analogues. Br. J. Pharmacol. 117(3):493-501, 1996a.

Gouarderes, C., Kar, S., Zajac, J.-M., Neuroscience, 74, 21-27, 1996b.

Gouarderes, C., Sutak, M., Zajac, J. M., and Jhamandas, K. Antinociceptive effects of intrathecally administered F8Famide and FMRFamide in the rat. Eur. J. Pharmacol. 237(1):73-81, 1993.

Guarneri, L, Ibba, M, Angelico, P, Colombo, D, Fredella, B and Testa, R. Effects of drugs used in the therapy of detrusor hyperactivity on the volume-induced contractions of the rat urinary bladder. Pharmacolocical Research, 27: 173-187, 1993.

Hamann, L. G., et al, (1998) “Synthesis and Biological Activity of a Novel Series of Nonsteroidal,Peripherally Selective Androgen Receptor Antagonists Derived from 1,2-Dihydropyridono[5,6-g]quinolines”, J. Med. Chem. 41: 623-639.

Huang, E. Y.-K.; Li, J. Y.; Tan, P. P.-C.; Wong, C.-H.; Chen, J.-C. Peptides, 21, 205-210, 2000.

Hynes, J. B. and Campbell, J. P., (1997) “2-Amino-quinazolines”, J. Heterocycl. Chem. 34(2): 385-387.

Jhamandas, K., Sutak, M., Yang, H.-Y. T. Soc. Neurosci. 22, 1313, 1996.

Kavaliers, M., Colwell, D.D. Neuropeptide FF (FLFQPQRFamide) and IgG from neuropeptide FF antiserum affect spatial learning in mice. Neurosci. Lett. 157:75-78, 1993.

Kavaliers, M., Hirst, M., and Mathers, A. Inhibitory influences of FMRFamide on morphine- and deprivation-induced feeding. Neuroendocrinology. 40(6):533-535, 1985.

Kontinen, V. K., Aarnisalo, A. A., Idaenpaeaen-Heikkilae, J. J., Panula, P., and Kalso, E.

Neuropeptide FF in the rat spinal cord during carrageenan inflammation. Peptides 18(2):287-292, 1997.

Kuhla, D. E., et al, (1986) “Quinoline and Quinazoline Derivatives for Treating Gastrointestinal Motility Dysfunctions”, U.S. Pat. No. 4,563,460.

Labrouche, S., Laulin, J.-P., LeMoal, M., Tranu, G. and Simonnet, G. Neuropeptide FF in the rat adrenal gland: presence, distribution and pharmacological effects. J. Neuroendocrinology. 10: 559-565, 1998.

Laguzzi, R., Nosjean, A., Mazarguil, H., and Allard, M. Cardiovascular effects induced by the stimulation of neuropeptide FF receptors in the dorsal vagal complex: An autoradiographic and pharmacological study in the rat. Brain Res. 711(1-2):193-202, 1996.

Malin, D. H., Lake, J. R., Arcangeli, K. R., Deshotel, K. D., Hausam, D. D., Witherspoon, W. E., Carter, V. A., Yang, H. Y., Pal, B., and Burgess, K. Subcutaneous injection of an analog of neuropeptide FF precipitates morphine abstinence syndrome. Life Sci. 53(17):PL261-6, 1993.

Malin, D. H., Lake, J. R., Fowler, D. E., Hammond, M. V., Brown, S. L., Leyva, J. E., Prasco, P. E., and Dougherty, T. M. FMRF-NH2-like mammalian peptide precipitates opiate-withdrawal syndrome in the rat. Peptides 11(2):277-280, 1990.

Malin, D. H., Lake, J. R., Short, P. E., Blossman, J. B., Lawless, B. A., Schopen, C. K., Sailer, E. E., Burgess, K., and Wilson, O. B. Nicotine abstinence syndrome precipitated by an analog of neuropeptide FF. Pharmacol. Biochem. Behav. 54(3):581-585, 1996.

Morgan, D. G., Small, C. J., Abusnana, S., Turton, M., Gunn, I., Heath, M., Rossi, M., Goldstone, A. P., O'Shea, D., Meeran, K., Ghatei, M., Smith, D. M., and Bloom, S. The NPY Y1 receptor antagonist BIBP 3226 blocks NPY induced feeding via a non-specific mechanism. Regul. Pept. 75-76: 377-382, 1998.

Murase, T., Arima, H., Kondo, K., and Oiso, Y. Neuropeptide FF reduces food intake in rats. Peptides 17(2):353-354, 1996.

Muthal, A. V. and Chopde, C. T. Anxiolytic effect of neuropeptide FMRFamide in rats. Neuropeptides. 27: 105-108, 1994.

Muthal, A. V., Mandhane, S. N., and Chopde, C. T. Central administration of FMRFamide produces antipsychotic-like effects in rodents. Neuropeptides 31(4):319-322, 1997.

Oberling, P., Stinus, L., Le Moal, M., and Simonnet, G. Biphasic effect on nociception and antiopiate activity of the neuropeptide FF (FLFQPQRFamide) in the rat. Peptides 14(5):919-924, 1993.

Owens, M. J. Neurotransmitter receptor and transporter binding profile of antidepressants and their metabolites. J. Pharm. Exp. Ther. 283: 1305-1322, 1997.

Panula, P., Aarnisalo, A.A., and Wasowicz, K.

Neuropeptide FF, a mammalian neuropeptide with multiple functions [published erratum appears in Prog. Neurobiol. 1996 June; 49(3):285]. Prog. Neurobiol. 48(4-5):461-487, 1996.

Panula, P., Kivipelto, L., Nieminen, O., Majane, E. A., and Yang, H. Y. Neuroanatomy of morphine-modulating peptides. Med. Biol. 65(2-3):127-135, 1987.

Payza, K., Akar, C. A., and Yang, H. Y. Neuropeptide FF receptors: structure-activity relationship and effect of morphine. J. Pharmacol. Exp. Ther. 267(1):88-94, 1993.

Payza, K. and Yang, H. Y. Modulation of neuropeptide FF receptors by guanine nucleotides and cations in membranes of rat brain and spinal cord. J. Neurochem. 60(5):1894-1899, 1993.

Raffa, R. B. and Jacoby, H. I. FMRFamide enhances acetylcholine-induced contractions of guinea pig ileum. Peptides. 10: 693-695, 1989.

Pietra, C, Poggesi, E, Angelico, P, Guarneri, L and Testa R. Effects of some antidepressants on the volume-induced reflex contractions of the rat urinary bladder: lack of correlation with muscarinic receptors affinity. Pharmacological Research, 22: 421-432, 1990.

Raffa, R. B., Kim, A., Rice, K. C., de Costa, B. R., Codd, E. E., and Rothman, R. B. Low affinity of FMRFamide and four FaRPs (FMRFamide-related peptides), including the mammalian-derived FaRPs F-8-Famide (NPFF) and A-18-Famide, for opioid mu, delta, kappa 1, kappa 2a, or kappa 2b receptors. Peptides 15(3):401-404, 1994.

Robert, J. J., Orosco, M., Rouch, C., Jacquot, C., and Cohen, Y. Unexpected responses of the obese “cafeteria” rat to the peptide FMRF-amide. Pharmacol. Biochem. Behav. 34(2):341-344, 1989.

Roumy, M. and Zajac, J. M. Neuropeptide FF, pain and analgesia. Eur. J. Pharmacol. 345(1):1-11, 1998.

Swahn, B., et al, (1996) “2-Chloroquinolines”, Bioorg Med Chem Lett 6:14 pages 1635-1640.

Vilim, E. S., Ziff, E. Cloning of the neuropeptide NPFF and NPAF precursor form bovine, rat, mouse, and human. Soc. Neurosci. 21:760, 1995.

Wei, H., Panula, P., and Pertovaara, A. A differential modulation of allodynia, hyperalgesia and nociception by neuropeptide FF in the periaqueductal gray of neuropathic rats: Interactions with morphine and naloxone. Neuroscience 86(1):311-319, 1998.

Wong E. H., Knight A. R., Woodruff G. N.: [3H]MK-801 labels a site on the N-methyl-D-aspartate receptor channel complex in rat brain membranes. J Neurochem 50: 274-281, 1988.

DNA Encoding Mammalian Neuropeptide FF (NPFF) Receptors and Uses Thereof. PCT International Publication No. WO 00/18438.

Xu, M.; Kontinen, V. K.; Panula, P.; Kalso, E. Peptides, 10, 1071-1077, 1999.

Yang, H. Y. T., Martin, B. M. Soc. Neurosci. 21, 760, 1995.

Yang, H. Y., Fratta, W., Majane, E. A., and Costa, E. Isolation, sequencing, synthesis, and pharmacological characterization of two brain neuropeptides that modulate the action of morphine. Proc. Natl. Acad. Sci. U.S.A. 82(22):7757-7761, 1985.

Maggi, C. A., Furio, M., Santicioli, P., Conte, B. and Meli, A. Spinal and supraspinal components of GABAergic inhibition of the micturition reflex in rats. J Pharmacol Exp Ther 240: 998-1005, 1987.

Morikawa, K., Hashimoto, S., Yamauchi, T., Kato, H., Ito, Y. and Gomi, Y. Inhibitory effect of inaperisone hydrochloride (inaperisone), a new centrally acting muscle relaxant, on the micturition reflex. Eur J. Pharmacol. 213: 409-415, 1992.

What is claimed is:

1. A method of treating pain in a subject which comprises administering to the subject an amount of a compound effective to treat pain in the subject, wherein the compound binds to a NPFF1 receptor with a binding affinity greater than ten-fold higher than the binding affinity with which the compound binds to a NPFF2 receptor.

2. The method of claim 1, wherein the compound binds to the NPFF1 receptor with a binding affinity greater than 25-fold higher than the binding affinity with which the compound binds to a NPFF2 receptor.

3. The method of claim 2, wherein the compound binds to the NPFF1 receptor with a binding affinity greater than 50-fold higher than the binding affinity with which the compound binds to a NPFF2 receptor.

4. A method of treating a urinary disorder in a subject which comprises administering to the subject an amount of a compound effective to treat the urinary disorder in the subject, wherein the compound binds to a NPFF1 receptor with a binding affinity greater than ten-fold higher than the binding affinity with which the compound binds to a NPFF2 receptor.

5. The method of claim 4, wherein the urinary disorder is urinary incontinence.

6. The method of claim 5, wherein the urinary incontinence is urge incontinence or stress incontinence.

7. The method of claim 4, wherein the urinary disorder is urinary retention.

8. The method of claim 4, wherein the compound binds to the NPFFl receptor with a binding affinity greater than 25-fold higher than the binding affinity with which the compound binds to a NPFF2 receptor.

9. The method of claim 8, wherein the compound binds to the NPFF1 receptor with a binding affinity greater than 50-fold higher than the binding affinity with which the compound binds to a NPFF2 receptor.

10. The method of claim 1 or 4, wherein the subject is a human being and the NPFF1 receptor is the human NPFF1 receptor and the NPFF2 receptor is the human NPFF2 receptor.

11. The method of claim 1 or 4, wherein the compound is an agonist at the NPFF1 receptor and an agonist at the NPFF2 receptor.

12. The method of claim 1 or 4, wherein the compound is an antagonist at the NPFF1 receptor and an antagonist at the NPFF2 receptor.

13. The method of claim 1 or 4, wherein the compound is an agonist at the NPFF1 receptor and an antagonist at the NPFF2 receptor.

14. The method of claim 1 or 4, wherein the compound is an antagonist at the NPFF1 receptor and an agonist at the NPFF2 receptor.

15. The method of claim 1 or 4, wherein the compound binds to the human NPFF1 receptor with a binding affinity at least 10-fold higher than the binding affinity with which the compound binds to each of a human α_{1A} adrenoceptor, a human α_{1B} adrenoceptor, and a human α_{1D} adrenoceptor.

16. The method of claim 1 or 4, wherein the compound binds to the human NPFF1 receptor with a binding affinity at least 10-fold higher than the binding affinity with which the compound binds to each of a human α_{2A} adrenoceptor, a human α_{2B} adrenoceptor and a human α_{2C} adrenoceptor.

17. The method of claim 1 or 4, wherein the compound binds to the human NPFF1 receptor with a binding affinity at least 10-fold higher than the binding affinity with which the compound binds to a human dopamine D₂ receptor.

18. The method of claim 1 or 4, wherein the compound binds to the human NPFF1 receptor with a binding affinity at least 10-fold higher than the binding affinity with which the compound binds to a human histamine H₁ receptor.

19. The method of claim 1 or 4, wherein the compound binds to the human NPFF1 receptor with a binding affinity at least 10-fold higher than the binding affinity with which the compound binds to a human NMDA receptor.

20. The method of claim 1 or 4, wherein the compound binds to the human NPFF1 receptor with a binding affinity at least 10-fold higher than the binding affinity with which the compound binds to a human norepinephrine transporter or to a human serotonin transporter.

21. The method of claim 1 or 4, wherein the compound binds to the human NPFF1 receptor with a binding affinity at least 10-fold higher than the binding affinity with which the compound binds to each of a human neuropeptide Y1 receptor, a human neuropeptide Y2 receptor, a human neuropeptide Y4 receptor, and a human neuropeptide Y5 receptor.

22. A method of treating pain in a subject which comprises administering to the subject an amount of a compound effective to treat pain in the subject, wherein the compound binds to a NPFF2 receptor with a binding affinity greater than ten-fold higher than the binding affinity with which the compound binds to a NPFF1 receptor.

23. The method of claim 22, wherein the compound binds to the NPFF2 receptor with a binding affinity greater than 25-fold higher than the binding affinity with which the compound binds to a NPFF1 receptor.

24. The method of claim 23, wherein the compound binds to the NPFF2 receptor with a binding affinity greater than 50-fold higher than the binding affinity with which the compound binds to a NPFF1 receptor.

25. A method of treating a urinary disorder in a subject which comprises administering to the subject an amount of a compound effective to treat the urinary disorder in the subject, wherein the compound binds to a NPFF2 receptor with a binding affinity greater than ten-fold higher than the binding affinity with which the compound binds to a NPFF1 receptor.

26. The method of claim 25, wherein the urinary disorder is urinary incontinence.

27. The method of claim 26, wherein the urinary incontinence is urge incontinence or stress incontinence.

28. The method of claim 25, wherein the urinary disorder is urinary retention.

29. The method of claim 25, wherein the compound binds to the NPFF2 receptor with a binding affinity greater than 25-fold higher than the binding affinity with which the compound binds to a NPFF1 receptor.

30. The method of claim 29, wherein the compound binds to the NPFF2 receptor with a binding affinity greater than 50-fold higher than the binding affinity with which the compound binds to a NPFF1 receptor.

31. The method of claim 22 or 25, wherein the subject is a human being and the NPFF1 receptor is the human NPFF1 receptor and the NPFF2 receptor is the human NPFF2 receptor.

32. The method of claim 22 or 25, wherein the compound is an agonist at the NPFF1 receptor and an agonist at the NPFF2 receptor.

33. The method of claim 22 or 25, wherein the compound is an antagonist at the NPFF1 receptor and an antagonist at the NPFF2 receptor.

34. The method of claim 22 or 25, wherein the compound is an agonist at the NPFF1 receptor and an antagonist at the NPFF2 receptor.

35. The method of claim 22 or 25, wherein the compound is an antagonist at the NPFF1 receptor and an agonist at the NPFF2 receptor.

36. The method of claim 22 or 25, wherein the compound binds to the human NPFF2 receptor with a binding affinity at least 10-fold higher than the binding affinity with which the compound binds to each of a human α_{1A} adrenoceptor, a human α_{1B} adrenoceptor, and a human α_{1D} adrenoceptor.

37. The method of claim 22 or 25, wherein the compound binds to the human NPFF2 receptor with a binding affinity at least 10-fold higher than the binding affinity with which the compound binds to each of a human α_{2A} adrenoceptor, a human α_{2B} adrenoceptor and a human α_{2C} adrenoceptor.

38. The method of claim 22 or 25, wherein the compound binds to the human NPFF2 receptor with a binding affinity at least 10-fold higher than the binding affinity with which the compound binds to a human dopamine D₂ receptor.

39. The method of claim 22 or 25, wherein the compound binds to the human NPFF2 receptor with a binding affinity at least 10-fold higher than the binding affinity with which the compound binds to a human histamine H₁ receptor.

40. The method of claim 22 or 25, wherein the compound binds to the human NPFF2 receptor with a binding affinity at least 10-fold higher than the binding affinity with which the compound binds to a human NMDA receptor.

41. The method of claim 22 or 25, wherein the compound binds to the human NPFF2 receptor with a binding affinity at least 10-fold higher than the binding affinity with which the compound binds to a human norepinephrine transporter or to a human serotonin transporter.

42. The method of claim 22 or 25, wherein the compound binds to the human NPFF2 receptor with a binding affinity at least 10-fold higher than the binding affinity with which the compound binds to each of a human neuropeptide Y1 receptor, a human neuropeptide Y2 receptor, a human neuropeptide Y4 receptor, and a human neuropeptide Y5 receptor.
